(19) United States
(12) Patent Application Publication
(10) Pub. No.: US 2009/0028832 A1
(43) Pub. Date: Jan. 29, 2009

(54) Compositions and methods for targeted tumor therapy

(76) Inventors: Leland F. Chung, Atlanta, GA (US); Haiyen E. Zhau, Atlanta, GA (US)

Correspondence Address: MORRIS MANNING MARTIN LLP, 3343 PEACHTREE ROAD, NE, 1600 ATLANTA FINANCIAL CENTER, ATLANTA, GA, 30326, US

(21) Appl. No.: 11/891,361

(22) Filed: Aug. 10, 2007

Related U.S. Application Data
(60) Provisional application No. 60/836,650, filed on Aug. 10, 2006.
(60) Provisional application No. 60/842,010, filed on Sep. 5, 2006.

(51) Int. Cl. A61K 35/12; A61P 35/00

(52) U.S. Cl. 424/93.7

(57) ABSTRACT

The present invention relates to a method of treating a human patient having one or more tumor cells. In one embodiment, the method comprises the step of implanting at or around the site of one or more tumor cells in the patient a cell population comprising one or more prostate cancer cells characterized in that the one or more prostate cancer cells have the propensity of metastasizing to skeleton and soft tissues which represent one or more lethal phenotypes of human prostate cancer.

### CROSS-REFERENCE TO RELATED PATENT APPLICATION

This application claims the benefit, pursuant to 35 U.S.C. § 119(e), of U.S. provisional patent application Ser. Nos. 60/836,650, filed Aug. 10, 2006 entitled: COMPOSITIONS AND METHODS FOR TARGETED TUMOR THERAPY” and 60/842,010, filed Sep. 5, 2006, entitled “COMPOSITIONS AND METHODS FOR TARGETED TUMOR THERAPY” by Leland W. K. Chung and Haiyen E. Zhau, each of them is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### FIELD OF THE INVENTION

The present invention relates to compositions and methods for modulating expression of osteomimicry within tumor and tissue cells with calcification potential. The invention further relates to methods of using cancer cells to elicit specific immune responses to tumor and tissue cells with calcification potential. The invention further relates to use of such cancer cells in screening compounds that modulate expression of osteomimicry within tumor and tissue cells with calcification potential. Methods for using molecules and compounds identified by the screening assays for therapeutic treatments also are provided. The invention further relates to methods of treating tumors and other diseases and disorders involving tumor and tissue cells with calcification potential with compositions or compounds that modulate their osteomimetic potential.

### BACKGROUND OF THE INVENTION

### Cancer Bone Metastases

In 2004, bone metastases accounted for two thirds of an estimated 560,000 cancer deaths in the United States (1). Over 80% of cancer bone metastases come from prostate, breast, lung and renal cancers. Bone metastases often develop after patients fail hormonal therapy, are lethal, and have no effective therapy. Previous work targeting prostate cancer cells using conventional hormone therapy, chemotherapy or radiation therapy in men with hormonal refractory disease did not improve patient survival (2). New approaches targeting bone with zoledronic acid (Zometa) for breast and prostate cancers to slow down skeletal events in patients treated with hormonal therapy, bone-directed chemotherapy and radiation therapy using strontium-89 or samarium-153 for prostate and breast cancers have been approved by the Food and Drug Administration for the clinical treatment of osteoblastic/osteolytic bone metastases (3, 4). New chemotherapy modalities have shown promise for reducing the overall incidence of skeletal complications and improving survival in selected hormone-refractory prostate and breast cancer patients (5). These promising approaches are supported by laboratory results using gene therapy approaches to co-target tumor and stroma (6) and drug therapy targeting osteoblasts (7), osteoclasts (8); (9), marrow stromal cells (10-12), bone derived endothelium (13), cell adhesion to extracellular matrices (14) or selected growth factor pathways (15, 16), all of which have shown promise in a large number of bone metastasis models (8).

Bone is the second most common site of human cancer metastasis, harboring over 80% of the metastases from prostate, lung, breast and renal cancers (31). Bone metastasis contributes directly to cancer mortality and morbidity, with an estimated total of 560,000 deaths per year in the US and more than 85% of patients presenting with evidence of skeletal metastasis at autopsy (32). The extent of osseous involvement has been claimed to correlate directly with patient survival and the quality of life of cancer patients with bone pain, cancer-associated bone fractures and spinal compression, bone-metastasis-evoked cranial neuropathy from base of skull syndromes, anemia and infection (31). Despite some success transiently controlling the clinical symptoms with radiation, hormones, surgery and chemotherapy, advanced cancer inevitably becomes resistant to treatment. Recently, directly targeting the bone with bisphosphonate to slow bone turnover in prostate and breast cancer patients treated with hormone withdrawal therapy has been credited with reducing bone pain and skeletal complications (33). The use of strontium 89 in combination with chemotherapy for the treatment of hormone refractory prostate cancer has resulted in statistically significant increases in patient survival (34). The development of the endothelin-1 receptor antagonist, atrasentan, to control osteoblastic lesions associated with prostate cancer bone metastasis (35, 36), and the inclusion of thalidomide to control angiogenesis associated with tumor progression (37), have been used clinically to improve the control of cancer bone metastases.

### Tumor-Stroma Interaction

Tumor progression involves changes in genetic constituents as well as the gene expression of cancer cells. Changes are acquired through germ line transmission, somatic mutation and via epigenetic mechanisms through tumor-host interactions (38, 39). Laboratory and clinical data show that tumor-stroma interaction contributes to the development and progression of solid tumors (40). Cell culture studies, transgenic mouse models and carcinogenesis studies show that the progression of breast (41), prostate (42, 43), lung (44), skin (45) and gastric cancers (46) is promoted by stromal cells in the microenvironment surrounding the tumor. Using the human LNCaP prostate cancer cell line as a model, we demonstrated that prostate cancer is not a single-cell disease but involves intimate interaction between prostate cancer cells and prostate or bone stromal cells (47). Multiple early carcinogenetic steps defined at the genetic level can also be rationalized at the cellular level. For example, prostate cancer cells co-evolve with host stroma and both are needed for rapid tumor progression. Prostate cancer cells can evoke a “reactive stroma” response that drives further genetic and gene expression changes in prostate cancer (48). Through a series of complex, intimate bi-directional communications between prostate cancer and host stroma, which includes host fibromuscular stroma, endothelium, neural and infiltrating inflammatory cells, cancer cells gain additional growth and survival advantages and ultimately expand locally and disseminate to distant organs with lethal effect (49-51).

The inventors' previous studies (17, 23, 52) and others (18, 24, 53-57) show that human prostate cancer cells mimic osteoblasts by expressing proteins normally restricted to osteoblasts, such as osteocalcin (OC), bone sialoprotein (BSP), SPARC/osteonectin (ON), osteopontin (OPN) and the receptor activator of NF-κB ligand (RANKL). The metastatic human prostate cancer cells, C4-2 and C4-2B, also form bone when placed under mineralizing conditions (18). These results illustrate that human prostate cancer cells can functionally participate in the normal process of bone formation, bone turnover and mineralization like osteoblasts. Increasing osteoclastogenesis (creating bone pitting through increased bone turnover) could enhance the implantation and subsequent growth of prostate cancer cells in bone (58, 59).

Tissue Specific Expression within Tumor and Tissue Cells with Calcification Potential

The treatment of osteotropic tumors such as breast, osteosarcoma and prostate which have metastacised is a major challenge. These seemingly unrelated diseases, however, unite through a molecular analysis of the gene(s) that may be overexpressed in these forms of cancer during disease progression.

BSP is a noncollagenous bone protein in which tissue expression is limited to fully-differentiated osteoblasts in bone or other rare mineralized tissues, including tumors (Sodek J. et al., 1995, Conn. Tissue Research, 32:209-217). When evaluating human prostate cancer cells that have a propensity to metastasize to the skeleton, a surprising finding was that these cells have the ability to synthesize and secrete large amounts of non-collagenous bone matrix proteins, such as osteopontin (OPN) (Thalman G N, et al., 1997, Principles of Practice of Genitourinary Oncology, 409-416), osteocalcin (OC) (Curatolo C, et al., 1992, European Urology, 1:105-107), and BSP (Withold W., et al., 1997, Clinical Chemistry, 85-91). BSP is a 34 kilodalton protein rich in aspartic acid, glutamic acid, and glycine, with 50% of its carbohydrate rich in sialic acid. BSP is sulfated and phosphorylated (30% of serine residues), and contains a cell binding motiff sequence which is homologous to vitronectin (Oldbrg et al., 1988, J. Biol. Chem., 263:19433). BSP is involved in the nucleation front of mineralization during new bone formation, and binds hydroxyappatite tightly (Hunter et. al., 1993, PNAS, 90:8562; Chen et. al., 1992, JBMR, 7:987; Kobayashi et. al., 1996, J. Biochem., 119:475). Human BSP exists as a single copy gene on chromosome 4 (Fisher L. W. et. al., 1990, J. B. C., 265:4:2347-2351) and has 7 exons and 6 introns. BSP is distinct from other sialoproteins, such as, for example, dentin sialoprotein, osteopontin, IL-1, IL-6, TNF and other bone associated sialoproteins.

BSP is found in mature, bone-forming cells, but not in immature precursors (Bianco, et. al., 1991, Conn. Tiss. Int., 49:421; Chen et. al., 1991, Matrix, 11:133). BSP is found in trophoblastic cells of the placenta, and in cementum and dentin of teeth, but is absent in most other tissues, including unlineralized cartilage, intestine, kidney, liver, heart, and skeletal muscle (Macneil et. al., 1994, JBMR, 9:1597). In a transgenic mouse system, the activity of the BSP promoter was present at high levels in bone, but absent in kidney, liver, stomach, intestine, and spleen (Chen et. al., 1996, JBMR, 11:5:654-64), and the administration of exogenous glucocorticoid stimulated the expression of reporter gene 1.6 to 11 fold (Chen, et. al., 1996, Conn. Tiss. Res., 35:33-39). The DNA sequence of the BSP promoter is over 2000 base pairs long and contains numerous regulatory elements which include vitamin D, AP-1, glucocorticoid (GRE), hox, NF.kappa.b, TGF-.beta., CRE, etc. (Kim R. H., et. al., 1994, Matrix Biology. 14:3140; Sodek J., et. al., 1996, Connective Tissue Research, 35:23-31; Kim R. H., et. al., 1996, Biochem. J., 318:219-226; Yamauchi M., et. al., 1996, Matrix Biology, 15:119-130; Kerr J. M., et. al., 1997, Calcif. Tiss. Int., 60:276282; Ogata Y., et. al., 1995, European J. Biochem., 230:183-192).

### Osteocalcin

Osteocalcin (OC), a noncollagenous Gla protein produced specifically in osteoblasts, is synthesized, secreted, and deposited at the time of bone mineralization (Price, P. A. Vitamin-K dependent formation of bone GLA protein (onteocalcin) and its function. Vitam. Horm., 42:65-108, 1985). A recent study showed that immunohistochemical staining of OC as positive in primary osteoblastic osteosarcoma and chondroblastic osteosarcoma specimens as well as in five of seven fibroblastic osteosarcomas (Park, Y. K., Yung, M. H., Kim, Y. W., and Park, H. R. Osteocalcin expression in primary bone tumors: in situ hybridization and immunohistochemical study. J. Korean Med. Sci., 10:268-273, 1995). In addition, OC activity was detected in a wide spectrum of human tumors. This is consistent with the clinical observations that many human tumors exhibited calcification characteristics both in the primary and at distant metastases.

### β2M Biology and Cancer

β2M forms a small invariable light chain subunit of the class I major histocompatibility complex (MHC, or HLA in humans) on the cell membrane of all nucleated cells. During the continuous turnover of the MHC molecules, β2M is shed from the cell membrane into blood. Lymphocytes are the main source of serum free β2M (60). Serum or urine β2M concentration is increased in several malignant diseases including prostate cancer (25), myeloma (61), lung cancer (62), renal cancer (63), lymphocytic malignancies (64, 65) and some inflammatory and autoimmune disorders (66-68). In these malignancies, serum β2M has significant prognostic value. Interferons (IFNs) have the ability to enhance the expression of class I and II MHC molecules. Accordingly, IFNs cause a rise in the formation β2M, which helps to present MHC molecules onto cell membranes, decrease tumor evasiveness and thus enhance host defense mechanisms against tumor growth. IFN alpha is used in diseases like multiple myeloma, where serum β2M measurements can assess tumor burden. Since MHC presentation is associated with host acquired immunity (69), decreased β2M or lost MHC expression could contribute to tumor cells' evasiveness (70-72) as with enhanced engraftment in patients who received bone marrow transplantation (73). It should be stressed that increased β2M levels have direct growth-promoting effects on prostate cancer, myeloma and bone and dendritic cell growth (74-77). The mechanisms could involve the increased expression of IL 6, 8 and 10 by a number of cancer cell types (78, 79) bone-like proteins in prostate cancer cells (52), and critical growth factor receptors, notably type 1 and 2 IGF receptors and EGF receptor, that enhance tumor growth (80). Previous work on β2M in myeloma revealed that the concentrations of this protein in serum and bone marrow aspirate correlated inversely with patient prognosis (61).

In view of the above, it remains important to pursue new molecular pathways that can be used to improve prognosis and treatment of cancer patients with lethal cancer phenotypes, bone metastases and associated complications.

### SUMMARY OF THE INVENTION

The present invention focusses on how osteomimicry contributes to intracellular cell signaling through augmentation of soluble factors and extracellular matrix-mediated interaction between cancer and stroma in the tumor microenvironment. This invention addresses a long-felt need for safe and effective methods of treatment of cancers and disorders with calcification potential by providing compositions and methods for eliciting a specific immune response against cancer cells exhibiting epithelial to mesenchymal transition (EMT) and/or osteomimicry.

The invention disclosed herein is based upon the concept that the specific targeting of the process of osteomimicry, either alone or in combination with chemotherapy and/or radiation therapy, can prevent prostate, renal, breast, lung cancer cell growth and survival in bone through both direct and indirect actions on cancer cells and cells in the cancer microenvironment, and prolong the survival of cancer patients with bony and visceral organ metastases.

The invention disclosed herein is thus based in part upon the identification of novel targeted therapeutic agents for preventing, treating, curing and/or ameliorating tumors with calcification potential, including, but not limited to, localized or disseminated osteosarcoma, lung cancer, renal cancer, colon cancer, melanoma, thyroid cancer, brain cancer, multiple myeloma, and especially including, without limitation, breast and prostate cancers. The invention specifically targets sites of metastases of the above-mentioned osteotropic tumors, and where applicable, their supporting stroma in the metastatic environment. In addition, the present invention also relates to therapeutic agents which may also be applicable to benign conditions, such as benign prostatic hyperplasia (BPH) or arterial sclerotic conditions where calcification and/or mineralization occurs.

The invention also provides relevant biomarkers based on the understanding of molecular pathways leading to cancer bone and visceral organ metastasis. The assessment of combinations of biomarkers relating to the process of osteomimicry is predictive of cancer dissemination prior to radiographic and biochemical evidence of such events.

The invention is premised upon the discovery that certain genes (list in Appendix A) that may be differentially expressed by certain cancer cells (ARCaP_{E}, ARCaP_{M} cells or second generation subclones thereof) specifically cause these cells to home in on bone and/or visceral organs. It is therefore postulated that by presenting these unique markers expressed by any one or more of the aforementioned genes in the host through the delivery of said cells either alone, or with stroma in the metastatic environment, as immune elicitors which are predicted to mount a strong immune response in the host that results in specific immunity directed against the survival of cancer cells in the host. Representative non limiting examples of such immune elicitors may be found in Table 1, Appendix A, Appendix B. Additional examples of such immune elicitors include, but are not limited to, RANKL, RANK, ADAM9, EMT related genes (vimentin, N-cadherin, IL13Rα2), and the process of osteomimicry related genes, such as VEGF, neuropilin, neuropilin modifiers, pCREB, pERK, pAkt, Shh and Gli).

In one aspect of the present invention, the invention comprises an immunotherapy vaccine for the treatment of cancer in a patient comprising cells from a first cancerous cell line obtained from a metastasis of prostate cancer exhibiting epithelial to mesenchymal transition (EMT) with high degree of invasion, migration and metastasis to bone and visceral organs.

In another aspect of the present invention, the invention comprises the use of the aforementioned immunotherapy vaccines for the treatment of cancer in a patient wherein the vaccine further comprises use of cells with a broad-spectrum of antigenic epitopes, including the co-inoculation of the said cells with PC-3 and/or LNCaP cells and/or cells in the cancer microenvironment.

In one embodiment, the first cancerous cell line is allogeneic, syngeneic, xenogeneic, autologous, heterologous, or any combination thereof.

In one embodiment, the cancer being treated is cancer of the bone, kidney, bladder, breast, heart, brain, thyroid, adrenal gland, lymph node, liver, lung, myeloma, B cell lymphoma, osteosarcoma, melanoma, liver or GI tumors or any combination thereof.

In another aspect of the present invention, the invention further comprises an immunotherapy vaccine for the treatment of cancer in a patient comprising cells from a first cancerous cell line obtained from a metastasis of prostate cancer exhibiting epithelial to mesenchymal transition (EMT) with high degree of invasion, migration and metastasis to bone and visceral organs, coupled with normal non-cancerous cells from one or more surrounding tissues. In one embodiment, the normal non-cancerous cells are allogeneic, syngeneic, xenogeneic, autologous, heterologous, or any combination thereof.

In one aspect of the present invention, the invention comprises an immunotherapy vaccine for the treatment of cancer in a patient comprising cells from a first cancerous cell line obtained from a metastasis of prostate cancer exhibiting epithelial to mesenchymal transition (EMT) with high degree of invasion, migration and metastasis to bone and visceral organs, wherein the cancerous cells further contain or express an immune elicitor (representative non limiting examples of such immune elicitors may be found in Table 1, Appendix A, Appendix B. Additional examples of such immune elicitors include but are not limited to RANKL, RANK, ADAM9, EMT related genes (vimentin, N-cadherin, IL 3Rα2), and the process of osteomimicry related genes, such as VEGF, neuropilin, pCREB, pERK, pAkt, Shh and Gli). Other potential immune elicitors include, for example, inter alia, GM-CSF, PSA, acid phosphatase, PSMA, etc.)

In yet another aspect of the present invention, the invention comprises an immunotherapy vaccine for the treatment of cancer in a patient comprising cells from a first cancerous cell line obtained from a metastasis of a cancer (for example, but not limited to, prostate cancer) exhibiting epithelial to mesenchymal transition (EMT) with high degree of invasion, migration and metastasis to bone and visceral organs and one or more host cells from the host microenvironment comprising host stromal cells, including endothelial cells, fibroblasts, reactive stromal cells (defined as altered stromal cells in contact with transformed cancerous epithelial cells, inflammatory cells, smooth muscle cells, inter alia, or any combination thereof.

In yet another embodiment, the invention further comprises an immunotherapy vaccine wherein the host microenvironment in bone comprises stromal cells exhibiting different characteristics such as, for example, but not limited to marrow stromal cells, osteoclasts, osteoblasts, macrophages, monocytes, bone marrow associated endothelial cells, and/or pericytes or any combination thereof.

In yet another embodiment, the invention further comprises an immunotherapy vaccine wherein the host microenvironment in bone comprises stromal cells in primary prostate cancer such as, for example, but not limited to, smooth muscle cells, fibroblasts, reactive stromal cells, myofibroblasts, neuroendocrine cells, endothelial cells, inflammatory cells (inter alia, lymphocytes, leukocytes, NK cells, macrophages), basal cells, and/or stem cells or any combination thereof.

In yet another aspect of the present invention, the invention comprises an immunotherapy vaccine for the treatment of cancer in a patient comprising cells from a first cancerous cell line obtained from a metastasis of cancer (for example, but not limited to, prostate cancer) exhibiting epithelial to mesenchymal transition (EMT) with high degree of invasion, migration and metastasis to bone and visceral organs and one or more host cells comprising host stromal cells, including endothelial cells, fibroblasts, reactive stromal cells (defined as altered stromal cells in contact with transformed cancerous epithelial cells, inflammatory cells, smooth muscle cells, etc or any combination thereof, wherein the cells or one or more host cells contain or express an immune elicitor.

In yet another aspect of the present invention, the invention comprises an immunotherapy vaccine for the treatment of cancer in a patient comprising cells from a first cancerous cell line obtained from a metastasis of cancer (for example, but not limited to, prostate cancer) exhibiting epithelial to mesenchymal transition (EMT) with high degree of invasion, migration and metastasis to bone and visceral organs, wherein the cancerous cell line is fused with other host cells, including host stromal fibroblasts, myofibroblasts, stem cells, muscle cells, genetically modified cells, or any combination thereof.

In yet another embodiment, the invention further comprises anyone of the aforementioned immunotherapy vaccines wherein the cancerous cell line or fused cancerous cell line is further tagged with a fluorescent dye, marker protein and/or genetic material or any combination thereof.

In one embodiment, the cancerous cell line is ARCaP, ARCaP_{E}, ARCaP_{M}, ARCaP_{Ad}, or a second-generation thereof that has exhibited epithelial to mesenchymal transition (EMT)-induced transdifferentiation from ARCaP_{E}, or a second-generation thereof, or any combination thereof. Each of the aforementioned cancerous cell lines are slow, fast-growing or a combination thereof second generation derivatives of Applicants' parental ARCaP cell line as previously patented in U.S. Pat. No. 5,874,305 (the entire contents of which are incorporated herein by reference in their entirety). Samples of the ARCaP, ARCaP_{E} ARCaP_{M}, ARCaP_{M} and the parental ARCaP cell lines are available through the inventors. Each of the cell lines are monitored, and have been maintained, and are continuous from the original specimen. The parental ARCaP cell line has also been deposited with the American Type Culture Collection, Rockville, Md., under Accession Number ATCC CRL-12277 (deposited Jan. 24, 1997).

In another embodiment, the ARCaP, ARCaP_{E} ARCaP_{M}, ARCaP_{M} or a second-generation thereof that has exhibited epithelial to mesenchymal transition (EMT)-induced transdifferentiation from ARCaP_{E}, or a second-generation thereof or any combination thereof may be co-administered with PC-3 and/or LNCaP cells to a patient in need thereof.

In yet another aspect of the present invention, the invention comprises the aforementioned immunotherapy vaccines wherein the host cell line is derived from a cancer (for example, but not limited to, prostate cancer) that has metastasized to one or more of bone, kidney, bladder, breast, heart, brain, thyroid, adrenal gland, lymph node, liver, lung, myeloma, B cell lymphoma, osteosarcoma, melanoma, liver and GI tumors or any combination thereof. The hypothesis is that when a patient is immunized with a cancer cell that is capable of residing in the host metastatic site(s), such as bone and visceral organs, plus cells in the cancer cell microenvironment, such immunization serves to induce an immune response that rejects the tumor growth in bone and visceral organs.

In yet another aspect of the present invention, the invention comprises any one of the aforementioned immunotherapy vaccines wherein the cell line further contains or expresses an osteomimicry or osteomimicry process-related genes or gene products that confer on the cells the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL), VEGF, Neuropilin, Neuropilin modifiers, IL13Rα2 and/or to increase bone turnover through Epithelial to Mesenchymal Transition (EMT). Expression of such osteomimicry or osteomimicry process-related genes or gene products by the cells serves to induce a specific immune response that rejects the tumor growth in bone and visceral organs.

In yet another aspect of the present invention, the invention further comprises anyone of the aforementioned immunotherapy vaccines, wherein the cell line is to be administered with an additional agent comprising an β2m siRNA, a β2m antibody, a GPCR antagonist, a PKA/CREB signal activation interrupter, a selective agent that interferes with β2 m/PKA/CREB signaling, a selective agent that interferes with CREB transcription factor and complex formation, or any combination thereof. Such gene products are expected to interfere with or disrupt the ability of the cancer cell to gain increased metastatic potential thereby preventing or markedly reducing the cancer cell's ability to exhibit Epithelial to Mesenchymal Transition (EMT) and/or exhibit osteomimetic properties.

In yet another aspect of the present invention, the invention further comprises anyone of the aforementioned immunotherapy vaccines, wherein the cell line further contains or expresses an osteomimecry regulatory region sequence, or transcriptionally active fragment thereof of one or more osteomimecry target genes including, but not limited to, genes that are related to or downstream from the VEGF axis, AR axis, GPCR axis, PKA/CREB axis, genes depicted in Appendix A, or any combination thereof, wherein said osteomimecry regulatory region sequence can regulate the activity or activities of one or more of said genes by interfering with the osteomimetic potential of said osteotropic cells. Representative non-limiting examples of Putative cAMP/PKA downstream genes in prostate cancer cells include those listed in Table 1 below.

In yet another aspect of the present invention, the invention further comprises anyone of the aforementioned immunotherapy vaccines, wherein the vaccine composition for the treatment of cancer further comprises the immunogenic agent a physiologically acceptable excipient, adjuvant or carrier or any combination thereof.

In yet another aspect of the present invention, the invention further comprises anyone of the aforementioned immunotherapy vaccines, wherein the adjuvant comprises one or more of bacille Calmette-Guerin, a Mycobacterium, Mycobacterium vaccae, Tetanus toxoid, Diphtheria toxoid, Bordetella Pertussis, interleukin 2, interleukin 12, interleukin 4, interleukin 7, Complete Freund's Adjuvant, Incomplete Freund's Adjuvant, and non-specific adjuvant.

In yet another aspect of the present invention, the invention further comprises anyone of the aforementioned immunotherapy vaccines, wherein the adjuvant comprises inactivated Mycobacterium vaccae bacilli.

In yet another aspect of the present invention, the invention further comprises anyone of the aforementioned immunotherapy vaccines, wherein the adjuvant comprises inactivated bacilli Calmette-Guerin.

In yet another aspect of the present invention, the invention further comprises anyone of the aforementioned immunotherapy vaccines, wherein the cell line and/or its mixture with other cells in cancer metastatic microenvironment, is lethally irradiated to ensure that the cells are replication incompetent.

In yet another aspect of the present invention, the invention comprises the aforementioned immunotherapy vaccines, wherein the cell line is irradiated utilizing gamma irradiation at 20-400 Gy, 50 to 300 Gy, or 100 to 150 Gy, or any combination thereof.

In yet another aspect of the present invention, the invention comprises the aforementioned immunotherapy vaccines, further comprising a cryoprotectant.

In yet another aspect of the present invention, the invention comprises the aforementioned immunotherapy vaccines, wherein the cryoprotectant comprises at least one of 10-30% v/v aqueous glycerol, 5-20% v/v dimethyl sulphoxide and 5-20% w/v human serum albumin.

In yet another aspect of the present invention, the invention comprises the aforementioned immunotherapy vaccines, wherein the cell line or strain (strain includes those cells grown out of the host prior to establishing a line) of any one of the aforementioned cancerous cell lines or second generation variants thereof further comprises cells directly derived from the host patient, such as the patient's stromal fibroblasts from either primary or metastatic sites, cultured from patient's biopsy specimens. In another embodiment, the invention further comprises the use of a stock of cell strains from patients' metastatic biopsy specimens, such as bone and visceral organs, mixed with the cells from the cancerous cell line ARCaP as an adjuvant or co-booster) wherein the cell is transfected with a potential immune elicitor, such as GM-CSF, PSA and other potential osteomimicry and/or osteomimicry process related genes.

In yet another aspect of the present invention, the invention comprises the aforementioned immunotherapy vaccines, wherein the vaccine is administered intradermally.

In yet another aspect of the present invention, the invention comprises the aforementioned immunotherapy vaccines, wherein the vaccine is administered intra-prostatically.

In yet another aspect of the present invention, the invention further comprises a method of inhibiting progression of cancer in a patient, comprising administering to the patient in need thereof of an effective amount of anyone of the aforementioned immunotherapy vaccines.

In yet another aspect of the present invention, the invention further comprises a method of inhibiting progression of cancer that has metastasized to a tissue selected from the group consisting of bone, kidney, bladder, breast, heart, brain, thyroid, adrenal gland, lymph node, liver, lung, myeloma, B cell lymphoma, osteosarcoma, melanoma, liver and GI tumors in a patient, comprising administering to the patient an effective amount of anyone of the aforementioned immunotherapy vaccines.

In yet another aspect of the present invention, the invention further comprises methods of drug screening by incorporating any one or more of the aforementioned cell lines or second generations thereof as vehicles for conducting drug screening to identify compounds, synthetic or natural, for the prevention of cancer metastasis or eradication of pre-existing cancer cells in mammalian skeleton. In yet another aspect of the present invention, the invention further comprises methods of drug screening employing improved methodologies so as to provide non-invasive visualization of tumors in mammals employing quantum dot technology, bioluminescence, fluorescence-tagged cells, MRI, PET, CT or micro-CT and/or the use of 3-D culture and co-culture models with relevant stromal cells or any combination thereof.

In yet another aspect of the present invention, the invention further comprises methods of screening of lethal phenotype-associated genes and incorporating any one or more of the aforementioned cell lines or second generations thereof using protein chip-based technologies routinely available in the art. In certain embodiments, the screening of lethal phenotype-associated genes includes those genes that are related to bone responsive to cancer colonization in bone (such as increased bone turnover via increased osteoblastogenesis and osteoclastogenesis), genes related to increased cell survival or antiapoptosis and increased cell proliferation, genes related to the induction of cachexia responses, genes related to the induction of new microvessel or vessel formation, genes promote cell cycle progression, genes resist to stress responses such as induced by hypoxia, osmolarity, pH and reactive oxygen species changes.

In yet another aspect of the present invention, the invention further comprises methods of screening using posttranlational changes of the proteins encoded by such lethal phenotype-associated genes, including, but not limited to, glycosylation, phosphorylation, acetylation, and/or RNA changes (for example, microRNA) and incorporating any one or more of the aforementioned cell lines or second generations thereof.

In yet another aspect of the present invention, the invention further comprises a method for identifying a compound which modulates the osteomimetic potential of a cell comprising target genes downstream from the induction of osteomimicry related genes or genes downstream from G protein coupled receptor/Protein kinase A/cyclic AMP responsive element binding protein (defined here as osteomimetic potential): (a) contacting a test compound with anyone of the aforementioned cancerous cells that exhibit osteomimetic potential; (b) measuring expression of one or more osteomimetic gene products or potential in the cell; and (c) comparing the level of expression of one or more osteomimetic gene products and potential in the cell in the presence of the test compound to a level of expression of one or more osteomimetic gene products in the cell in the absence of the test compound; wherein, if the level of the expression of one or more osteomimetic gene products in the cell in the presence of the test compound differs from the level of expression of the one or more osteomimetic gene products or potential in the cell in the absence of the test compound, a compound that modulates expression of the one or more osteomimetic gene products or potential is identified.

In one aspect of the present invention, a method is provided for treating and/or ameliorating an osteotropic-related disease or proliferative disorder in a mammal comprising administering to a subject in need thereof a therapeutically effective amount of a composition comprising anyone of the aforementioned cancerous cells and a pharmaceutically acceptable excipient, wherein said composition elicits a specific immune response against the osteomimetic potential and/or the osteomimetic properties of any known disease or disorder with osteomimetic or calcification and/or mineralization potential.

The anti-osteomimetic or osteomimicry interfering compound or a functional derivative thereof that exhibits anti-osteomimetic or osteomimicry interfering activity can include, but is not limited to, small organic molecules including naturally-occurring, synthetic, and biosynthetic molecules, small inorganic molecules including naturally-occurring and synthetic molecules, natural products including those produced by plants and fungi, nucleic acids, chemically modified nucleic acids, peptides, chemically modified peptides, and proteins. The anti-osteomimetic or osteomimicry interfering agent has the capability of inhibiting the osteomimetic potential and/or the osteomimetic properties of any known disease or disorder with osteomimetic or calcification and/or mineralization potential.

In another aspect of the present invention, a method is provided for interfering with the osteomimetic properties of a cell comprising introducing into a cell of a subject in need thereof an osteomimecry interfering compound, wherein said compound prevents or ameliorates the expression of the osteomimetic properties of cancer cell in the patient.

In one embodiment of the present invention, a method is provided for interfering with the osteomimetic properties of a cancer cell comprising introducing into anyone of the aforementioned cancerous cells of a subject in need thereof an osteomimecry interfering compound, wherein said compound prevents or ameliorates the expression of the osteomimetic properties of cancer cells in the patient.

In yet another aspect of the present invention, a method is provided for interfering with the osteomimetic potential of a cancer cell comprising introducing into anyone of the aforementioned cancer cells of a subject in need thereof of an osteomimecry interfering compound, wherein said compound interferes with the osteomimetic potential of cancer cells in the patient, prevents its growth, abrogates its supportive blood vessels, eliminates the survival androgen receptor signaling and causes massive cell death in pre-existing cancers in the subject, or any combination thereof.

In one embodiment of the latter aspect of the present invention, the osteomimicry interfering compound inhibits one or more determinants governing prostate cancer bone colonization wherein said determinants comprise prostate cancer cell adhesion, extravasation, migration, invasion and interaction with bone cells or any combination thereof.

In yet another aspect of the present invention, a method is provided for identifying osteomimetic related and/or downstream genes using any one of the aforementioned cancer cells and assessing the levels of expression and the variant forms of osteomimetic related and/or downstream genes as effective diagnostics for use in methods for predicting cancer bone and visceral organ metastases prior to radiographic and biochemical evidence of such events.

In each of the aforementioned aspects and embodiments of the present invention, the osteomimicry interfering compound interferes with the ability of a cell or cancer cell to express highly restricted bone-like proteins comprising one or more of osteocalcin (OC), bone sialoprotein (BSP), SPARC/osteonectin (ON), osteopontin (OPN) and the receptor activator of NF-κB ligand (RANKL), and/or to increase calcification, mineralization and bone turnover through the expression of genes normally restricted to osteoblasts and through epithelial to mesenchymal transition (EMT), a basic biologic process associated with increases of the motility, migration, invasion and survival of the cell or the cancer cell.

In each of the aforementioned aspects and embodiments of the present invention, the osteomimicry interfering compound comprises a compound whose osteomimicry-specific target is a gene or gene product from the vascular endothelial growth factor (VEGF) axis, a gene or gene product from the androgen receptor (AR) axis, a gene or gene product from the 7 transmembrane G protein-coupled receptor (GPCR) axis, a gene or gene product from the Protein Kinase A (PKA)/cyclic AMP responsive element binding protein (CREB) axis, and those current genes or gene products identified by microarray analyses as shown in shown in Appendix A, or any combination thereof.

In each of the aforementioned aspects and embodiments of the present invention, the osteomimicry interfering compound comprises a beta 2 microglobulin (β2M) siRNA, a β2M antibody, a Runx2 (i.e., cbfa1) transcription factor-specific siRNA, antibody, or antagonist, a GPCR antagonist, an AR or signaling antagonist, a VEGF or signaling antagonist, a PKA/CREB signal activation interrupter, a selective agent that interferes with β2M/PKA/CREB signaling, a selective agent that interferes with CREB transcription, phosphorylation and complex formation, a selective agent that interferes with β2M complex formation with either an intracellular protein or a membrane receptor or any combination thereof.

In one embodiment of the present invention, β2M siRNA encapsulated in liposomes, either with or without a targeting ligand (such as for example, and not by way of limitation, PSMA Ab, or aptamer), β2M siRNA or ribozyme viral vectors may be used to inhibit prostate cancer growth by inhibiting cancer cell proliferation and also the growth of neighboring stromal cells that support cancer growth, including osteoblasts, marrow stromal cells, endothelial cells and inflammatory cells.

In yet another embodiment of the present invention, viral vectors may used for the construction and delivery of therapeutic genes under the control of tissue-specific and tumor-restrictive promoters to co-target tumor and stroma. Representative examples of viral vectors and tissue-specific and tumor-restrictive promoters that may be used for the construction and delivery of therapeutic genes under the control of tissue-specific and tumor-restrictive promoters may be found in published U.S. Patent Application Nos. 20020025307, 20030078224, and 20040101840, and in U.S. Pat. No. 6,596,534, the contents of each which are incorporated herein by reference in their entirety. Such viral vectors may additionally used to study the targeting of β2M-mediated signaling pathways and their regulation of cancer growth and bone metastasis.

In each of the aforementioned aspects and embodiments of the present invention, the osteomimicry interfering compound is administered either alone with anyone of the aforementioned immunotherapy vaccines or in combination with agents that target in parallel with osteomimicry interfering compounds, such as the use of compounds that block VEGF axis, AR axis, GPCR axis, PKA/CREB axis, genes depicted in Appendix A and any combination thereof. Representative, non-limiting examples of compounds that block VEGF axis, AR axis, GPCR axis, PKA/CREB axis may be found in Appendix B infra. In addition, this concept of tumor targeting using any one of the aforementioned cancerous cells can be extended to target cells in the microenvironment (such as, for example, and not by way of limitation, fibromuscular stromal cells, bone marrow stromal cells, endothelial cells and inflammatory cells) and in further combination with cytotoxic (for example, and not by way of limitation, paclitaxel, doxorubicin, cisplatin, mitoxantrone, estramustine, etoposide, ketoconazole vinblastine) and ionizing radiation agents (for example, and not by way of limitation, strontium-89, Yttrium-90 or Lu-177 tagged Abs).

In each of the aforementioned aspects and embodiments of the present invention, the administration of the osteomimicry interfering compound in conjunction with any one of the aforementioned cancer cells to a subject in need thereof has several beneficial effects including, inter alia, to block or retard cancer and benign growth, to reduce bone turnover for the treatment of osteoporosis, to prevent tumor progression through immune induced inhibition of epithelial to mesenchymal transition (EMT) and development of hormone independent disease, to induce massive apoptosis in cancer but not in normal cells, to facilitate bone marrow engraftment and transplantation, or any combination thereof, and to eliminate vascular plaque and calcification for the treatment of cardiovascular diseases.

In yet another aspect of the present invention, a method is provided for treating and/or ameliorating an osteotropic-related cancer or proliferative disorder comprising introducing into a subject in need thereof any one of the aforementioned cancerous cells transduced with a vector comprising a regulatory region sequence, or transcriptionally active fragment thereof of one or more osteomimecry target genes including, but not limited to, genes that are related to or downstream from the VEGF axis, AR axis, GPCR axis, PKA/CREB axis, or any of the genes depicted in Appendix A, or any combination thereof, wherein said osteomimecry regulatory region sequence can regulate the activity or activities of one or more of said genes by interfering with the osteomimetic potential of a cancer.

In one embodiment of the aforementioned methods, the cancer or other proliferative disorder is selected from the group consisting of osteosarcoma, prostate cancer, breast cancer, colon cancer, lung cancer, renal cancer, brain cancer, multiple myeloma, thyroid cancer, melanoma or any other disease consisting of benign prostate hyperplasia (BPH), vascular plaque formation in cardiovascular conditions or disorders with excessive calcification and mineralization potential, or any combination thereof.

In another embodiment of the aforementioned method, the osteotropic-related disease or proliferative disorder comprises osteoporosis, increased bone turnover through enhanced interaction between RANK and RANKL, and increased cancer bone colonization through enhanced osteomimicry and recruitment of host cells that promote osteoclastogenesis and osteoblastogenesis.

In yet another aspect of the present invention, a screening method is provided for identifying a compound which modulates the osteomimetic potential or properties of a cell comprising: (a) contacting a test compound to a cell that exhibits osteomimetic potential or properties; (b) measuring expression of one or more osteomimetic gene products in the cell; and (c) comparing the level of expression of one or more osteomimetic gene products in the cell in the presence of the test compound to a level of expression of one or more osteomimetic gene products in the cell in the absence of the test compound; wherein, if the level of the expression of one or more osteomimetic gene products in the cell in the presence of the test compound differs from the level of expression of the one or more osteomimetic gene products in the cell in the absence of the test compound, a compound that modulates the osteomimetic potential or properties of a cell is identified.

In one embodiment of the aforementioned screening method, the cell that exhibits osteomimetic potential comprises a cancer cell from osteosarcoma, prostate, breast, colon, lung, brain, renal, multiple myeloma, thyroid, melanoma or any other known disease or disorder with osteomimetic or calcification potential, or any combination thereof.

In another embodiment of the aforementioned screening method, the test compound comprises an osteomimicry interfering compound that interferes with the ability of the cell or cancer cell to express highly restricted bone-like proteins comprising one or more of osteocalcin (OC), bone sialoprotein (BSP), SPARC/osteonectin (ON), osteopontin (OPN) and the receptor activator of NF-κB ligand (RANKL), and/or to increase bone turnover through epithelial to mesenchymal transition (EMT), or any combination thereof.

In another embodiment of the aforementioned screening method, the test compound comprises a β2M siRNA, a β2M antibody, a Runx2 (i.e., cbfa1) transcription factor-specific siRNA, antibody, or antagonist, a GPCR antagonist, an AR or signaling antagonist, a VEGF or signaling antagonist, a PKA/CREB signal activation interrupter, a selective agent that interferes with β2M/PKA/CREB signaling, a selective agent that interferes with CREB transcription, phosphorylation and/or complex formation, a selective agent that interferes with β2M complex formation with other intracellular proteins, or any combination thereof.

In another embodiment of the aforementioned screening method, the osteomimicry interfering compound is administered in combination with one or more more antagonists interfering with AR axis (see Appendix B), interfering with GPCR axis (see Appendix B), one or more anti-angiogenic agents (see Appendix B), one or more cytotoxic drugs (for example, and not by way of limitation, paclitaxel, doxorubicin, cisplatin, mitoxantrone, estramustine, etoposide, ketoconazole vinblastine), or any combination thereof.

For each of the above-recited methods of the present invention, the therapeutically effective amount of one or more substances exhibiting anti-osteomimetic or osteomimetic interfering activity or a functional derivative thereof may be administered to a subject in need thereof in conjunction with a therapeutically effective amount of one or more anti-microbacterial drugs and/or inflammatory compounds and/or a therapeutically effective amount of one or more immunomodulatory agents.

In certain embodiments of the method of the present invention, the anti-inflammatory compound or immunomodulatory drug comprises interferon; interferon derivatives comprising betaseron, beta.-interferon; prostane derivatives comprising iloprost, cicaprost; glucocorticoids comprising cortisol, prednisolone, methyl-prednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in combination.

In one embodiment, the reduction or inhibition of pain and/or symptoms associated with one or more of each of the above-recited cancers or proliferative disorders is on the order of about 10-20% reduction or inhibition. In another embodiment, the reduction or inhibition of pain is on the order of 30-40%. In another embodiment, the reduction or inhibition of pain is on the order of 50-60%. In yet another embodiment, the reduction or inhibition of the pain associated with each of the recited cancers or proliferative disorders is on the order of 75-100%. It is intended herein that the ranges recited also include all those specific percentage amounts between the recited range. For example, the range of about 75 to 100% also encompasses 76 to 99%, 77 to 98%, etc, without actually reciting each specific range therewith.

In yet another aspect, the present invention is directed to a method of relieving or ameliorating the pain or symptoms associated with any one or more of the above-identified cancers or proliferative disorders in a mammal suffering from any one or more of the above-identified cancers or proliferative disorders which comprises administering to the mammal in need thereof a therapeutically effective pain or symptom-reducing amount of a pharmaceutical composition comprising effective amounts of a substance exhibiting anti-osteomimetic or osteomimetic interfering activity in conjunction with any one of the aforementioned cancerous cells, either alone or in combination with one or more anti-inflammatory compounds or immunomodulatory agents; and a pharmaceutically acceptable carrier or excipient, wherein said anti-osteomimetic or osteomimetic interfering substance or compound is sufficient to inhibit the osteomimetic property and/or potential of said cancer or proliferative disorder.

The present invention also relates to the combined use of the pharmaceutical composition exhibiting anti-osteomimetic or osteomimetic interfering activity in conjunction with any one of the aforementioned cancerous cells, in combination with one or more antibacterial or antiviral compositions or any combination thereof for treating any one of the aforementioned cancers or proliferative disorders, or any combination thereof.

The present invention provides methods for therapeutically or prophylactically treating cancers or proliferative disorders in a subject.

The method for therapeutically treating cancers or proliferative disorders comprises the step of administering pharmaceutically effective amounts of a compound or substance exhibiting anti-osteomimetic or osteomimetic interfering activity or derivative thereof to the subject in conjunction with any one of the aforementioned cancerous cells, after occurrence of the cancers or proliferative disorders.

The method for prophylactically treating cancers or proliferative disorders comprises the step of administering pharmaceutically effective amounts of a compound or substance exhibiting anti-osteomimetic or osteomimetic interfering activity or derivative thereof to the subject in conjunction with any one of the aforementioned cancerous cells, prior to the occurrence of the cancers or proliferative disorders.

Either methodology inhibits the cancers or proliferative disorders.

The present invention also provides compositions and methods for screening compounds that modulate expression within osteotropic cells and tissues. In particular, it provides compositions comprising polynucleotide sequences from osteomimecry regulatory region polynucleotide sequences or transcriptionally active fragments thereof, as well as nucleic acids that hybridize under highly stringent conditions to such polynucleotide sequences, such as for example, and not by way of limitation, osteocalcin promoter sequence (SEQ ID NO. 1), bone sialoprotein (SEQ ID NO. 2), SPARC/osteonectin promoter sequence (SEQ ID NO. 3), osteopontin promoter sequence (SEQ ID NO. 4), the receptor activator of NF-κB ligand promoter sequence (SEQ ID NO. 5), and the androgen receptor promoter sequence (SEQ ID NO. 6), and use of those polynucleotide sequences to screen compounds that modulate expression within osteotropic cells and tissues and/or that interfere with the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL).

Specifically also provided are expression vectors comprising one or more of the aforementioned osteomimicry regulatory region sequence, and/or transcriptionally active fragments thereof, operably associated to a heterologous reporter gene, e.g., luciferase, and host cells and transgenic animals containing such vectors. The invention also provides methods for using such vectors, cells and animals for screening candidate molecules for agonists and antagonists of osteotropic-related disorders. Methods for using molecules and compounds identified by the aforementioned screening assays for therapeutic treatments also are provided.

In another embodiment, the transgenic animal models of the invention can be used for in vivo screening in conjunction with any one of the aforementioned cancerous cells, to test the mechanism of action of candidate drugs for their effect on osteotropic-related disorders. Specifically, the effects of drugs on osteotropic-related cancers or disorders can be assayed including, for example, but not limited to, localized or disseminated osteosarcoma, lung cancer, colon cancer, thyroid cancer, brain cancer, melanoma, multiple myeloma, and especially including, without limitation, breast, lung, renal, and prostate cancers, in benign conditions such as BPH or arterial sclerotic conditions, where calcification and mineralization occurs, and in bone marrow or stem cell transplantation, where increased osteomimicry signaling via cAMP/PKA/CREB could prevent engraftment. Therapeutic drugs that interfere with this signaling will increase the efficiency and success of bone marrow or stem cell transplantation.

For example, and not by way of limitation, a composition comprising a reporter gene is operatively linked to an osteomimecry regulatory region sequence, or transcriptionally active fragment thereof such as for example, and not by way of limitation, osteocalcin (OC), bone sialoprotein (BSP), SPARC/osteonectin (ON), osteopontin (OPN) and the receptor activator of NF-κB ligand (RANKL). The osteomimicry regulatory region sequence, or transcriptionally active fragment thereof driven reporter gene is expressed as a transgene in animals. The transgenic animal, and cells derived from osteotropic cells of such a transgenic animal, can be used to screen for candidate compounds that interfere with the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL).

In addition, the serum and/or circulating cells from such transgenic animals can be used for the assay and serve as the end points to define the effects of osteomimicry interfering drugs in experimental animals. Moreover, serum and/or circulating cells from such experimental animals and/or from human patients could also be used as diagnostic indicators since they will reflect the status of osteomimicry and hence predict the ability of cancer cells to home to the skeleton and visceral organs. The analyses of osteomimicry related target genes and gene products in serum and other biologic fluids and tissue samples can also be used to predict the therapeutic response of patients to therapy.

Without being bound by any particular theory, such anti-osteomimetic or osteomimicry interfering compounds or functional derivatives thereof in conjunction with any one of the aforementioned cancerous cells, are likely to interfere with the function of trans-acting factors, such as transcription factors, cis-acting elements, such as promoters and enhancers, as well as any class of post-transcriptional, translational or post-translational compounds involved in osteotropic-related disorders. As such, they are powerful candidates for treatment of such disorders, including, but not limited to, localized or disseminated osteosarcoma, lung cancer, renal cancer, colon cancer, melanoma cancer, thyroid cancer, brain cancer, multiple myeloma, and especially including, without limitation, breast and prostate cancers, and benign conditions, such as BPH or arterial sclerotic conditions where calcification occurs, and bone marrow and stem cell transplantation where increased osteomimicry may prevent the engraftment of foreign cells to the immune intact host. The compounds of the invention additionally can be used in conjunction with any one of the aforementioned cancerous cells, to interfere with the expression of crucial growth and differentiation-associated genes such as growth factors, growth factor receptors, non-collagenous bone matrix proteins, bone morphogenic proteins, host immune regulatory molecules, etc, for repairing the damages acquired during aging and/or degenerative conditions.

In one embodiment, the invention provides methods utilizing any one of the aforementioned cancerous cells for high throughput screening of compounds that modulate specific expression of genes within osteotropic cells and tissues. In this aspect of the invention, cells from osteotropic-cells or tissues, are removed from a transgenic animal or directly established from human cancer and non-cancer tissues, and cultured in vitro. The expression of a reporter gene is used to monitor osteotropic-specific gene activity. In a specific embodiment, luciferase is the reporter gene. Compounds identified by this method can be further tested for their effect on osteotropic-related disorders in experimental animal models with defined conditions that mimic human diseases as transgenes or as transplanted xenografts.

In each of the aforementioned aspects and embodiments of the present invention, due to the tissue specificity of the anti-osteomimecry regulatory region sequence, or transcriptionally active fragment thereof, the anti-osteomimicry regulatory region sequence, or transcriptionally active fragment thereof therapeutically active agents of the present invention are effective in conjunction with any one of the aforementioned cancerous cells, not only when administered via direct application, such as by injection, but also when administered systemically to the body in conjunction with any one of the aforementioned cancerous cells, via intravenous administration, intra-arterial administration, intra-tumoral administration, perfusion, oral administration or the like, because gene expression will be limited and localized to specific, cell and tissue types, including, but not limited to, osteoblasts and osteoblast-mimicking cancer and benign cells, osteotropic benign and cancer cells. Furthermore, since many of the therapeutic agents of the invention exhibit pleiotropic effects and targeting selectively cells dependent upon osteomimicry to grow and survive, expression of the therapeutic agents in only specifically targeted cells is essential in order to prevent numerous, harmful side effects to normal cells. A representative example of a harmful site effect includes the development of autoimmune diseases in the host.

As described in more detail herein, an anti-osteomimecry regulatory region sequence, or transcriptionally active fragment thereof can comprise any number of configurations based upon the promoter sequences and/or enhancer sequences for osteocalcin (OC), bone sialoprotein (BSP), SPARC/osteonectin (ON), osteopontin (OPN) and the receptor activator of NF-κB ligand (RANKL), including, but not limited to, a promoter or fragment thereof; an enhancer or fragment thereof or enhancer-like sequence or fragment thereof; a silencer or fragment thereof; a promoter or fragment thereof and a enhancer or fragment thereof or enhancer-like sequence or fragment thereof; a promoter and a heterologous enhancer or fragment thereof; a heterologous promoter or fragment thereof and a enhancer or fragment thereof or enhancer-like sequence or fragment thereof; and multiple copies of promoters, enhancers or fragments thereof; and multimers of the foregoing.

Methods are also provided herein for measuring the activity of an anti-osteomimecry regulatory region sequence, or transcriptionally active fragment thereof based upon available promoter sequences and enhancer sequences for osteocalcin (OC), bone sialoprotein (BSP), SPARC/osteonectin (ON), osteopontin (OPN) and the receptor activator of NF-κB ligand (RANKL), and thus for determining whether a candidate anti-osteomimetic or osteomimicry interfering compound or a functional derivative thereof has the ability to modulate an anti-osteomimicry regulatory region sequence, or transcriptionally active fragment thereof and/or has the ability to modulate the osteomimetic properties and/or potential of a cancer or proliferative disorder.

In one embodiment, the anti-osteomimecry regulatory region sequence, or transcriptionally active fragment thereof based upon the promoter and enhancer or enhancer-like sequence of one or more of available promoter sequences and enhancer sequences for osteocalcin (OC), bone sialoprotein (BSP), SPARC/osteonectin (ON), osteopontin (OPN) and the receptor activator of NF-κB ligand (RANKL) may be in any orientation and/or distance from the coding sequence of interest, and may comprise multimers of the foregoing, as long as the desired inhibition or interruption of cell-specific transcriptional activity is obtained. Transcription activation or inhibition of transcriptional activation can be measured in a number of ways known in the art (and as described in more detail below), but is generally measured by detection and/or quantitation of mRNA or the protein product of the coding sequence under control of (i.e., operatively linked to) a transcriptional regulatory sequence. As discussed herein, an anti-osteomimecry regulatory region sequence, or transcriptionally active fragment thereof can be of varying lengths, and of varying sequence composition.

In one aspect of the invention, the pharmaceutical compositions of the present invention are administered orally, systemically, via an implant, intravenously, topically, intrathecally, intracranially, intraventricularly, by inhalation or nasally.

In yet another embodiment of the method of the present invention, the osteomimicry related and downstream target genes are expected to be expressed in the blood circulation, in other biologic fluid and/or biopsy specimens. Assessment of the level of expression of these gene products has prognostic value in predicting the expression of one or more lethal phenotypes by cancer cells. These non-invasive methods are expected to be more sensitive than the existing radiographic or biochemical procedures which fail to distinguish cancer cells with different malignant and metastatic potential.

In certain embodiments of the methods of the present invention, the subject or mammal is a human.

In other embodiments of the methods of the present invention, the subject or mammal is a veterinary and/or a domesticated mammal.

There has been thus outlined, rather broadly, the important features of the invention in order that a detailed description thereof that follows can be better understood, and in order that the present contribution can be better appreciated. There are additional features of the invention that will be described hereinafter.

In this respect, before explaining at least one embodiment of the invention in detail, it is to be understood that the invention is not limited in its application to the details as set forth in the following description and figures. The present invention is capable of other embodiments and of being practiced and carried out in various ways. Additionally, it is to be understood that the terminology and phraseology employed herein are for the purpose of description and should not be regarded as limiting.

As such, those skilled in the art will appreciate that the conception, upon which this disclosure is based, can readily be used as a basis for designing other methods for carrying out the several features and advantages of the present invention. It is important, therefore, that the claims be regarded as including such equivalent constructions insofar as they do not depart from the spirit and scope of the present invention.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1. Histomorphology of ARCaP cell subclones ranged from cobble-stone shaped ARCaP_{E} to spindle-shaped ARCaP_{M} cells.

FIG. 2. ARCaP_{M} cells exhibit higher invasion (A), migration (B), and growth rate (C) than ARCaP_{E}.

FIG. 3. Conditioned medium derived from fast-growing ARCaP_{M} subclone stimulated the growth of slow-growing ARCaP_{E} cells.

FIG. 4. Protein expression profile changes from ARCaP_{E} to ARCaP_{M} and ARCaP_{Ad} are closely associated with epithelial to mesenchymal transition.

FIG. 5. Histomorphology (top panel) and vimentin expression (IHC, bottom panel) of primary tumors of ARCaP_{E}, ARCaP_{M}, metastatic bone and adrenal gland induced by intracardiac injections of ARCaP_{E} cells in athymic mice.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise.

Additionally, some terms used in this specification are more specifically defined below, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification. Furthermore, subtitles may be used to help a reader of the specification to read through the specification, which the usage of subtitles, however, has no influence on the scope of the invention. Additionally, certain theories are proposed and disclosed herein; however, in no way they, whether they are right or wrong, should limit the scope of the invention.

As used herein, “about” or “approximately” shall generally mean within 20 percent, preferably within 10 percent, and more preferably within 5 percent of a given value or range. Numerical quantities given herein are approximate, meaning that the term “about” or “approximately” can be inferred if not expressly stated.

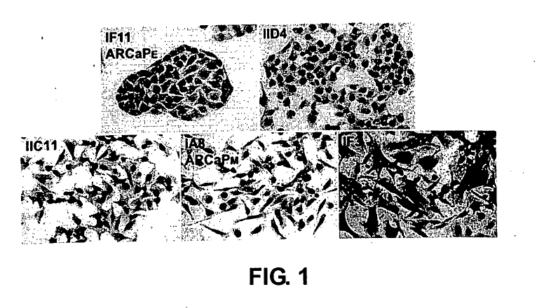

The description will be made as to the embodiments of the present invention in conjunction with the accompanying drawings of FIGS. 1-5. In accordance with the purposes of this invention, as embodied and broadly described herein, this invention, in one aspect, relates to the fact that the inventors identified a novel molecular target, osteomimicry, which confers the ability of prostate cancer cells to mimic the gene expression and behaviors of osteoblasts, thus allowing prostate cancer cells to adhere to bone cells and grow and survive in bone. Osteomimetic prostate cancer cells express not only highly restricted bone-like proteins such as osteocalcin (OC), bone sialoprotein (BSP) osteopontin (OPN) and receptor activator of NF-κB ligand (RANKL), they are also capable of forming mineralized bone under certain cell culture conditions. Bone matrix proteins are highly expressed in both localized and metastatic prostate cancers but not in normal prostate.

### Rationale for Osteomimicry as a Novel Cancer Target

Osteomimicry, defined as the ability of non-malignant benign cells [benign prostate hyperplasia and fibromuscluar stromal cells around the blood vessels to grow and proliferate] or cancer cells to mimetic the gene expression and behaviors of bone cells thereby allowing the cancer cells to grow, survive and invade in the bone microenvironment Osteomimicry may also regulate host immunity and other immune status.

Osteomimicry is controlled by: 1) the cAMR/PKA/CREB pathway which is intimately tied to GPCR-mediated downstream signaling, AR axis, VEGF axis, EMT, integrin-ECM signaling; 2) the Runx2/cbfa1 signaling pathway, and controls the ability of cancer cells and cells in cancer microenvironment to grow, to undergo apoptosis, to gain survival advantages, to invade, to migrate or to metastize and differentiate. Osteomimicry is responsible for the synthesis, secretion and deposition of the bone like proteins: OC, OPN, ON, BSP and RANKL by benign and cancer cells.

Osteomimicry is responsible for the up and down regulation of a series of genes related to the control of cell growth, cell death, oxidative stress, cell differentiation and cell cycle progression. These genes can be regulated to affect the fate of benign and cancer cells (see Appendix A).

Osteomimicry occurs in normal cells which allows them to calcify and mineralize, providing a foundation for the development of BPH and athleroschlerotic plaques. Osteomimicry affects the presentation of MHC class-1 antigen in normal cells and effects the immunity and immune status of the host.

Osteomimicry has dual functions: 1) when overexpressed expressed in benign or cancer cells, increased growth survival and decreased apoptosis of cells are expected. By antagonizing osteomimicry through the use of osteomimetic interfering drugs, we expect an inhibition of the growth and increased apoptosis of benign or cancer cells in the host, 2) when overexpressed in normal host cells there is enhanced host immunity, thus decreased efficiency of bone marrow and stem cell engraphments. This condition can be reversed through the admistration of osteomimetic interfering compounds or compositions.

Compositions or compounds that interfere with osteomimicry in cancer and benign cells can block cancer progression by causing massive cancer cell death, abrogating neovascular endothelial sprouting and ingrowth of endothelium into the invasive tumor, preventing EMT, inhibiting attachment of cancer cell to selected ECM and attenuating cancer cell survival. These drugs are also expected to decrease calcification and mineralization of normal benign cells and cause apoptotic death of BPH and fibromuscular smooth muscle cells.

Compositions or compounds that interefere with osteomimicry include, but are not limited to those compounds or drugs identified in Appendix B, as well as those compounds known to interefere with the Runx2 signaling pathway. These drugs are expected to inhibit osteomimicry, resulting in decreased benign and cancer cell growth, and improved efficiency of bone marrow and stem cell transplantation.

Compositions or compounds that interfere with osteomimicry can be used in combination with other cytotoxic drugs and/or radiation therapies that could work either additively or synergistically to enhance the pharmacologic affect of osteomimitic interfering compositions or compounds.

Compositions or compounds interfering with osteomimicry can be used either alone or in combination (as described above) for the inhibition of use in the control of the growth and metastasis of cancer cell which include but not limit to prostate, breast, multiple myeloma, renal, lung, brain, thyroid, colon, and osteosarcoma, the abnormal growth of the benign cell which include but not limit to smooth muscle and fibroblast related to mesenchymal lineage in the benign conditions such as BPH and atherosclerosis, and the host immunity during bone marrow and stem cell transfer.

Biomarkers (see Appendix A, bone matrix proteins and signal components involved in AR axis, VEGF axis, GPCR axis, cAMP/PKA/CREB axis, and Runx2 signalling pathways as described above) in the biologic fluids or tissues related to osteomimicry are predictors for cancer, bone and visceral organ metastases, the lethal phenotypes of cancers.

Osteomimicry is determined by a soluble factor, b2m or b2m like protein or peptide. B2m is secreted by cancerous or normal cells with the ability to activate downstream target genes (see Appendix A bone matrix proteins and signal components involved in AR axis, VEGF axis, GPCR axis, cAMP/PKA/CREB axis, and Runx2 signalling pathways as described above) through activation of transcription of, but not limited to CREB.

Osteomimicry can be assayed by transfecting a target cell with an osteomimicry target gene promoter reporter construct, either alone or in combination with a host of other osteomimicry target gene promoter reporter constructs, and the extent of osteomimicry in a normal condition is expected to be in proportion with the activation of these promoter reporter constructs in a target cell. However, it is also expected that variations can occur due to heterogeneity of transcription factors, modifiers and interactive proteins in cells so that basal osteomimicry status and its responsiveness to regulators are expected to be varied in a cell context dependent manner.

Compositions or compounds that interfere with osteomimicry in a typical assay include but are not limited to the assessment of osteomimicry related genes, such as human OC promoter luciferase activity, either alone or in combination with a series of other osteomimicry related gene promoter reporter constructs, in a target cell are effective agents for the clinical use in the control of the growth and metastasis of cancer cell which include but not limit to prostate, breast, multiple myeloma, renal, lung, brain, thyroid, colon, and osteosarcoma, the abnormal growth of the benign cell which include but not limit to smooth muscle and fibroblast related to mesenchymal lineage in the benign conditions such as BPH and atherosclerosis, and the host immunity during bone marrow and stem cell transfer.

Compositions or compounds interfering with osteomimicry may include, but not limited to small molecules, antibodies, nucleic acids and naturally occurring pharmaceuticals which can be assayed to interfere with osteomimicry by interfering promoter reporter activity, cell growth, cell survival, apoptosis, cell invasion, cell migration and cell spreading.

Compositions or compounds interfering with osteomimicry may include, but not limited to nucleotide sequences or their fragments that recognize the critical promoter regions that regulate target downstream from osteomimicry which include, but not limited to AR axis, VEGF axis, GPCR axis, cAMP/PKA/CREB axis, and Runx2 signalling pathways and genes described in Appendix A.

Compositions or compounds interfering with osteomimicry may include, but not limited to analogs of small molecules that interfere with the AR axis, GPCR axis, VEGF axis, and PKA/CREB axis as exhibited in the accompanying figures. Representative, non-limiting examples of selective agents that interfere with PKA/CREB signal activation include those selective agents that target the specific region of the cis-element in hOC promoter, located between −643 to −636 (CRE), which the inventors have shown is responsible for conferring cAMP regulation of hOC promoter activity in human prostate cancer cells. Additional representative, non-limiting examples of selective agents that interfere with PKA/CREB signal activation include those selective agents that target other regions of CRE within hBSP promoter, −79 to −72 (CRE1) and −674 to −667 (CRE2), that must also be activated upon the exposure of human prostate cancer cells to cAMP mimetics and yet unidentified growth factor(s) in the CM of prostate cancer and bone stromal cells.

Antibodies interfering with osteomimicry may include, but not limited to specific binders or interference molecules as a protein, peptide, nucleic acid, radioactive/cytotoxic derivatives that inter with osteomimicry related downstream signaling.

What follows is a detailed description of the osteomimicry-specific polynucleotides and nucleic acids of the invention (for example, and not by way of limitation, osteomimicry regulatory region sequences, and transcriptionally active fragments thereof), in conjunction with reporter constructs utilizing such osteomimicry-specific polynucleotides and nucleic acids can then be used to screen for candidate compounds or substances that interfere with the expression of the heterologous coding sequence. Such identified compounds or substances that interfere with osteomimicry regulatory region sequence, and transcriptionally active fragments thereof will be likely candidate compounds that interfere with the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL).

### Osteomimecry Polynucleotides and Nucleic Acids of the Invention

The present invention encompasses polynucleotide sequences comprising the 5′ regulatory region, and transcriptionally active fragments thereof, of an osteomimicry gene, including, for example, and not by way of limitation, osteocalcin (OC), bone sialoprotein (BSP), SPARC/osteonectin (ON), osteopontin (OPN), and the receptor activator of NF-κB ligand (RANKL). The nucleotide sequences of the promoter regions of each of osteocalcin (OC) (SEQ ID NO. 1), bone sialoprotein (BSP) (SEQ ID NO. 2), SPARC/osteonectin (ON) (SEQ ID NO. 3), osteopontin (OPN) (SEQ ID NO. 4), the receptor activator of NF-κB ligand (RANKL) (SEQ ID NO. 5), and the androgen receptor (AR) (SEQ ID NO. 6) are depicted below. The promoter sequences of VEGF, NP-1 and Runx2 are available in the public domain and one of ordinary skill in the art may obtain the promoter sequences of VEGF, NP-1 and Runx2 and use such promoter sequences in the methods of the present invention without undue experimentation.

| | |
|---|---|
| hOC promoter (0.9 kb) | |
| GAGCTCGGATCCACTAGTAACGGCCGCCAGTGTGCTGGAATTCCCCTT | (SEQ ID NO. 1) |
| CTGCAGGGTCAGGAGGAGAATCGTGGGGCCAGGAGGGCAGAGGCACACTCC | |
| ATCTTCGTGCTCCTCACAGGCCCTGCTCCCTGCCTGCTAAGACACAGGGAAGG | |
| GGGCCCCCACCTCAGTGCCTCCCTCCCTTCCCTGTGCCTGTGTACCTGGCAGT | |
| CACAGCCACCTGGCGTGTCCCAGAAACCAACCGGCTGACCTCATCTCCTGCCC | |
| GGCCCCACCTCCATTGGCTTTGGCTTTTGGCGTTTGTGCTGCCCGACCCTTTCT | |
| CCTGTCCGGATGCGCAGGGCAGGGCTGAGCCGTCGAGCTGCACCCACAGCAG | |
| GCTGCCTTTGGTGACTCACCGGGTGAACGGGGGCATTGCGAGGCATCCCCTCC | |
| CTGGGTTTGGCTCCTGCCCACGGGGCTGACAGTAGAAATCACAGGCTGTGAG | |
| ACAGCTGGAGCCCAGCTCTGCTTGAACCTATTTTAGGTCTCTGATCCGCGCTT | |
| CCTCTTTAGACTCCCCTAGAGCTCAGCCAGTGCTCAACCTGAGGCTGGGGGTC | |
| TCTGAGGAAGAGTGAGTTGGAGCTGAGGGGTCTGGGGCTGTCCCCTGAGAGA | |
| GGGGCCAGAGGCAGTGTCAAGAGCCGGGCAGTCTGATTGTGGCTCACCCTCC | |
| ATCACTCCCAGGGCCCCTGGCCCAGCAGCCGCAGCTCCCAACCACAATATCCT | |
| TTGGGGTTTGGCCTACGGAGCTGGGGCGGATGACCCCAAATAGCCCTGGCAG | |
| ATTCCCCCTAGACCCGCCCGCACCATGGTCAGGCATGCCCCTCCTCATCGCTG | |
| GGCACAGCCCAGAGGGTATAAACAGTGCTGGAGGCTGGCGGGGCAGGCCAG | |
| CTGAGTCCTGAGCAGCAAAGGGCGAATTCTGCAGATATCCATCACACTGGCG | |
| GCCGCT | |
| hBSP promoter (1.5 kb) | |
| GTGGCACATATACACGATGGAATACTATGCAGCCATAAAAAATGATG | (SEQ ID NO. 2) |
| AGTTCATGTCCTTTGTGGGGACATGTATGAAATTGGAAACCATCATTCTCAGT | |
| AAACTATCACAAGAACAAAAAACCAAACACCACATATTCTCACTCATAGGTG | |
| GGAATTGAACAATGAGATCACATGGACACAGGAAGGGGAACATCACACTCTG | |
| GGGACTGTTGTGGGGTGGGGGGAGTAAGGGGAGGGATAGCATTGGGAGATA | |
| TACCTAATGCTAGATGATGAGTTAGTGGGTGCAGCACACCAGCATGGCACAT | |
| GTGTACGTATGTAACTAACCTGCACACAATGTGCACATGTACCCTAAAACTTA | |
| AAGTATAATAATAAAAAAAATTAAGAGAAAAAAAGAAAAAAAATGATATTC | |
| ATTAATTTTTGATTTCTCAAGCAGACTTCGCAACTGGAGGAAGAATAAAATGA | |
| CTAGACTAGGAGAATATGCAAACTATTAAGCTAGATTTCCCTTTATAAATTAA | |
| AAAATTAGTACTTTAGTTTATCAATCCATTCTTTGTGGTGTTGGTTTCATGAAT | |
| CATTTCAAAAACAATGGATCACTCCTGCTAGCTCTAGTCATTTTGTTATTCTCA | |
| TAGGAAAAAAATTAAATATGAAAATGAATAGAAAAGATATATATAGAAGCC | |
| CAAGAAAAATCAGCTGACCTCACATGCACGACAGGAAGGCCACATAAATGG | |
| ACAATATACAGAGATTTAATTTACAAAACAAAATATAAAATCTGCCTCTCAGT | |
| GGTATGATTCTCAAAAGTTCTAACTTTTATACTCAGCATCATGTTTTAGCAACT | |
| ATATGTTACAAAGTCTGACCGACTTAATCATATCAACTTTAATTTATGAGTCA | |
| ATGAAGTATATTTCAGGAGGAAACATCAAATGATATTAAAATATTGATGGTT | |
| CATCTGCTGGTTTCCCTTATTATTTAGTTTTTCTTTCTTTTTTTAGCTAAACTAA | |
| TGTAAAAGTTATATCTAATGACAGCAAGCTTTCCTTTCTTTCGACATAGTGAA | |
| AACTTGTGTAATTATGAAATTTTTAAAAGGTTAAAGCCTTTGTTATTTATTTTA | |
| ATTCAAATCCAGTATATTATTATACATATTCGGAGCCCAAACTATTCATCTTC | |
| ATCTAAACCTTCAATTAAATTCCACAATGCAAACCTCTTGGCTCTAGAATCAC | |
| GTTTCTTGTTTATTCAACTGAGCCTGTGTCTTGAAAAAGTGTTGAAGTTTGGG | |
| GGTTTTCTGGTGAGAATCCACGTTCTGACATCACCTTGGTCGTGACAGTGATT | |
| GGCTGTTGGAAGGCAAAGAAGAGTTTATAGCCAGCAAGAGCAAGTGAATGA | |
| GTGAGTGAGAGGGCAGAGGAAATACTCAATCTGTGGCACTCACTGCCTTGAG | |
| CCTGCTTCCTCACTCCAGGACTGCCAGAGGGTAAGATTTAATAGAACAACGG | |
| hOPN promoter (2.2 kb) | |
| GGGAAGGAGA CAATAGTGTC AACTTGGGAT TGCCTAAGGC | (SEQ ID NO. 3) |
| AACAACAGAG CAAAACAAGA ACGCTTTGGT TCTCTGGGTC TCTGTCCCTG | |
| ATTGCATAGC GGGTCATTGT TGGGAAATAT TTCCTCACCT GGCATTCCAA | |
| GAAATGGTGA GCTCCACAGC TGTATATAGT CCTGTCATTA AATACAGGAG | |
| TGTTCTATCC CGCTGGAATT AAGAAAATTG GTAGAACCAG ATTGTGGTCT | |
| GAAATCTTTT TTCAGAAATG CTGCCATCGT GTGGCACTGC GGAGCTATGA | |
| CCAGAAGAGT CCTGTAAAGG GTCGTATGGT TCATCTCAAG ATGGCTGGGC | |
| TCCAGCATAA TCTATTCCTA TAATTAATTC TAGCTTCATA TTGAATCATT | |
| CCCGTGGGCA CAGAGTAAAC TACAGTAAAT CCTGTGGAAA TTTTGTTGTT | |
| TTTAGAATTT TCGGACTTCC CTCCACTAAA TTGACAACAT GACACGCTTA | |
| TGCGNGTATG TTTAAAGGAA AAAAATAGTT TTTAGAAGCA GAAAAAAGAA | |
| GTCTATTTTG CAACTTTATA ATCTGTGTGC TTNCTATTTT ATAGAGATAG | |
| TCGTCATCTT ACTTATTAAA ATGGGTGCTT ATTACCTACA AACCAATCAT | |
| ATCAATTCAT CTGGAATACA TCCAATTTAA GGGAGACATA TTTCCCCCTA | |
| CCAAATGTTC ATGAAACCTA TGAATTAGCT ATACACTATC ACTGCAAGAC | |
| ATTATTTAAT CTATATTTAT ATTAAAAGTA ATATTTGGCA AAAGGAAGCT | |
| GACACTTTAG GACTAATAAA AACCACAATT ACTTTTGCAG CAACCTAATA | |
| ATAAATAGGA CCATTTATTT TTCATCTCAA TTACACACAA GTCTTAACAA | |
| TAAAGGTGTA AGGTAAATAA ATAGTGCAAT CTGCATTTCA CAACTGAGAA | |
| GCAAATGAAG ATAAGTAATC TCAAGGCAAT ATTAAATATT TTAAAAGGAC | |
| CCAGAGCTCT GCTATCCCTG AATTCTGCTC TAATATTCGG ACTTTCCCTG | |
| TAATTTTCTT TCATTCAGAC ACCTTTTAAA TACCTAGTAA AGTGTTTTTT | |
| AATACAGAAA TTTTTAAAAA TGTTTTTCTT TTTAAGTGGC CTACTTTACA | |
| TACCTTGGGA GAAAAACTAG AAAAAAAGAT GATTCCAAAA TCGAATCTGT | |
| TCCTTTAGAA ATGTGCAAAA TTTCCTTATT GATGCATACA ATTTAAAGAT | |
| CTTACGTCTA CTCTCATTTT AATAACCTGT TCTTTTAAAG GACATTACAA | |
| TTCGTGACTG CCTGCCCCTC TTAAAAATTT CATAATAGTT AACACACATA | |
| TAGTCCTTAA GATACGCAGA GCATTTGCAT CTAATATGTG CTAAGCATTG | |
| CTAGTTTAAC ATACTAATTC ATTTAAACCC CTCAAAAACC CCATGACCTA | |
| GGTAATAGTA TTGCATTTCA TGGATGAGGG AACAAGGATA GGTAGGCTGG | |
| GCGATTTGCC CAAGGTTGCA CAGGTCAGCA GTGACACAGC GGAATTCAGA | |
| ACCACGGTCT GGCTCCTGAA GCAGCCCTCT CAAGCAGTCA TCCTTCTCTC | |
| AGTCAGAAAC TGCTTTACTT CTGCAACATC TAGAATAAAT TACCATTCTT | |
| CTATTTCATA TAGAATTTTA TATTTTAATG TCACTAGTGC CATTTGTCTA | |
| AGTAACAAGC TACTGCATAC TCGAAATCAC AAAGCTAAGC TTGAGTAGTA | |
| AAGGACAGAG GCAAGTTTTC TGAACTCCTT GCAGGCTTGA ACAATAGCCT | |
| TCTGGCTCTT CAATAAGTAC AATCATACAG GCAAGAGTGG TTGCAGATAT | |
| TACCTTTATG TTACTTAAAC CGAAAGAAAC AAAAATCCAT TGTATTTAAT | |
| TTTACATTAA TGTTTTTCCC TACTTTCTCC CTTTTTCATG GGATCCCTAA | |
| GTGCTCTTCC TGGATGCTGA ATGCCCATCC CGTAAATGAA AAAGCTAGTT | |
| AATGATATTG TACATAAGTA ATGTTTTAAC TGTAGATTGT GTGTGTGCGT | |
| TTTTGGTTTT TTTTTGTTTT AACCACAAAA CCAGAGGGGG AAGTGTGGGA | |
| GCAGGTGGGC TGGGCAGTGG CAGAAAACCT CATGACACAA TCTCTCCGCC | |
| TCCCTGTGTT GGTGGAGGAT GTCTGCAGCA GCATTTAAAT TCTGGGAGGG | |
| CTTGGTTGTC | |
| hON promoter (2.3 kb) | |
| GAATTCCTTGTACTTTTTTTCCCTTCTCAGTTCTGCACTTAACTCGTCTA | (SEQ ID NO. 4) |
| AAAAAATTAAAAAAGAATTTAAGAAACCACAAAGCTAAGCTGGGTGCGGTG | |
| GCTCACGCCTGTAATCCTAGCACTTTGGGAAGCCAAGGCATTCGGATTGCCCA | |
| AGCTCAGGAGTTCGAGACCAGCCTGGGCAACATGTTGAAACCCCATTTCTACT | |
| AAAAATACAATAAATTAGCTGGGTGTTGTGGCATGTGCGCCTGTAATCCCAG | |
| CTACTCTGGAGGCTGAGGCGCGATAATTGCTTGAACCCGGGAGGCAGAGGTT | |
| GCAGTGAGCCGAAATCATACCACTGCACTCCAGCCTGGGCGACAGAGTGAGT | |
| GAGACTCTGTCTCAAAACAAAACAAAACAAACAAACAAAAAAACCGGAAAC | |
| CAACAAAACTTTTTGAGGAACAAAGGGAACCAGGTATTTTATTAATTCTCATA | |
| CCTCCAGAGTGTTAGGCACAAAATAAACATTCAACCAAGACCTGTTGCACTG | |
| AGCAGTTCATATATAACAGGAGTGACCCAAGTTGAAACGTAGAATCAGCCCT | |
| CTCATACCACTTTTTGCCAGGTGATCATAGGCAAGTTACTTAGCATCTATGTTT | |
| CCTTATTATTAAAATGGTCATAATTACAATGCCTAAGATAAGGGGGTTGCTGT | |
| GAAGATTATTAAATCCTCAGTAAACTTTGGCTATTGTTACTCCTATGATTATC | |
| ATCAATATCATCAATTACCTTATCTGTTCAATACTGGTGGCACAGGTCCACCA | |
| GCTAGATGTCTAATCCCTTATGTGTCTATTAGTGGTACAAGTGGAGTTTGAGT | |
| GGGATTTTTTTTTTTTTTTTTAAGACCAGTTCCAAATCATCAAGGATGATACCA | |
| CTAGTAGCAGCTTGTCTTGTCTGTACAGTGGTAAGTCCTGGCCTTGCCTTTGTG | |
| GCAAATACAACCCCCTTGAATTGCTTGGCCCTTCTCAGCATTGCCTAATATTA | |
| GGGAGGACTCCTGTAAAGCTCACTGGTTAGAAGATCAAGACACTTGGGCCTG | |
| GTTCTGCCCCTGGGGGCCATTGGGTAATTCCTTGGAGTCTCCAGGCCTCACTT | |
| GCCCTCTGAACAAGAAAGAGGCCTGTTCTGGTCATCCCTCCAGCCTGTCCAGC | |
| CCTGGCACTCTGTGAGTCGGTTTAGGCAGCAGCCCCGGAACAGATGAGGCAG | |
| GCAGGGTTGGGACGTTTGGTCAGGACAGCCCACCGCAAAAAGAGGAGGAAA | |
| GAAATGAAAGACAGAGACAGCTTTGGCTATGGGAGAAGGAGGAGGCCGGGG | |
| GAAGGAGGAGACAGGAGGAGGAGGGACCACGGGGTGGAGGGGAGATAGAC | |
| CCAGCCCAGAGCTCTGAGTGGTTTCCTGTTGCCTGTCTCTAAACCCCTCCACA | |
| TTCCCGCGGTCCTTCAGACTGCCCGGAGAGCGCGCTCTGCCTGCCGCCTGCCT | |
| GCCTGCCACTGAGGTATGTGTGACCCCCGCCCAGCCTTTCCCTTCTATAGTTG | |
| CACCAACCCCGACACCCCCGTTCACGCCGTCAGCTCGTGTGCAAGGGAGGGA | |
| AGCTCTGCTGAGGATGCGCCTCTCCTCCCGGCTCCATCACGGCTCCCCTTAAG | |
| AGCATGGCCCTCGGTCCTGTCTGCCTGTTGCTTTTCAGAAGGTGGACTCACTG | |
| TGTAACTTTGTCTTCCCTTACAGGTTTACAGGAAAATAATCTCACTATGTTCTT | |
| CGGGGGAGCATTTTCTCACTCTCTGTTTTTCTCTGTGTCTGTCTCTGGTTTCAG | |
| AGGCTGCCTGCCTGTCCTCTTTGCTCCCTTTGCAAATGTGGCAGCCTCCTCCTT | |
| TCCTGGGAATCTGATCCCATCACAGCTGCCACAGGGACCTGGCCAGCAACCG | |
| GAGTCTGTCCTCCAGATCTCGGTCAGGGGTTCTGTTTTCCAAAAAGGGACTTT | |
| GCAGAACAATCAGTTGATCTCTGAAAGGGAAAGGGGGAGGCTTCACCATTAA | |
| TCCACACCTCTGGGAAGCTTCTGTTTTCCTCTAATTCTCCTCACTCCCAAACAC | |
| CACCTTCCGTCCCCCCAATACACAAATTTCAGCACCATTCTGCCTGAAATGGC | |
| ACCATCACAACCTCAGTCTTGGGTTAGGTGTTGTTCCTGTCCTGAGTTCCTTGG | |
| GATGGTAAACACAGGCAGTAGCCCTTAGTTTATCTAGATCTGAAAACCCAGA | |
| CATCAGATATCGTCAACCAAGACATGGGTGTAATGGGAGGTGGAGTGTGCTG | |
| GGGGAGATATTCTCAGAAGGGGGAAAGGGGGAAGGGAAGAGGGAGAGAATT | |
| C | |

### Human RANKL Promoter Sequence

| | | |
|---|---|---|
| 1 | acaccaaata tttataaata taactcacac aaataaaacc tctttggtgt tctcaaaatt | (SEQ ID NO. 5) |
| 61 | ttgaagaatg taaaaggttt gaaaattgct gatctagcaa atgactgaac atgaacagct | |
| 121 | atagtatttg tacctgccca gcagtgcagc aattccttat ccttctcata tctgcacttt | |
| 181 | aattttcctt tgacaaatat ctctccctcc tctcagccca tgacatgagg ttcacatggg | |
| 241 | gttaacttaa ttccctggct caaaggaaag gtattaaatt cagacttgta tccaaccatt | |
| 301 | cctgaagcta gacttagccc tatttttcaa taacatgaac caatcaattt tcacatgagt | |
| 361 | ccaaaataat tctatgttaa tacactaagg tactaggaaa tatagtttga gaaatgttga | |
| 421 | tccaaacatt gtgttattta cagtggagta ttgacataaa ctttgaatct tcaaatatgt | |
| 481 | tctggtgtct tggcatctct taatacctat tagcttacaa ggctttcact caactatttt | |
| 541 | ataattttga taatgactta attgattagt tgatatattg ttaaaataaa tatattaatg | |
| 601 | aatttatgat aaataaggca gataaataag acatgcaatt aggaagacat gttaaacaaa | |
| 661 | ttgttataat aatacaatca ctctcagctt aggatagctc ctggccactt tctctctggg | |
| 721 | tggtttttac tctgggagta gtttaaatca ttatctagta gtagtttaaa gcattatctt | |
| 781 | tgcctaagag ctttcgctga ctccccacat ttgcattgta ctaagagttt tctctgactc | |
| 841 | cccacatagg tctagaccct agtattataa gattctcatt gtacttgcac tttgccttca | |
| 901 | aagtactaat cacggttttg ttagtgattt gtgtgatgat ttgttgaatc tttttttttt | |
| 961 | tcccactagg gtgtaagccc catgttccat cttgatcacc atgtttctag cccagtgctg | |
| 1021 | gcatatagtg ggttctcact aatatatctg tagagtaaat gaagaaatgc atgagtgaca | |
| 1081 | tgacaggaga atttaaggat gccatgggag cataaaacag agggagccac ctgggtgagg | |
| 1141 | agagctgaga aagacttctg gagaggcgac atttgagctg agaaaggaaa gacaagtggg | |
| 1201 | agagtcctcc aggtgtagaa gttggagaga tgagcgctcc agttaggtag tatttgaagc | |
| 1261 | tgatgtagaa aaggagtctt gagccagctt gtgaaggact attggagagt tttattttta | |
| 1321 | tttttatctt ttttttaatt tttgagacag aatcttgctt tgtctcccag gctggagtgc | |
| 1381 | agtggcatga ttgtagctta ctgcagcttc gacctcctgg gctcaaacaa tccacctatc | |
| 1441 | tcagccttct gagtaactgg gaccagagat gtgcaccaaa atgcctggct aatttgttca | |
| 1501 | ttttttgtaa agatagggtc tccctatgtt ccccaggcta ttctccatct cctgggctcc | |
| 1561 | agtgatcctc acgcctcggc cacccaaagt gctgggatta tagaagtgaa ccactgcgcc | |
| 1621 | tggcctattg aaggttttta atcttcagag tttcgacttt atcaacaaca cttagaagcc | |
| 1681 | accaaagaat tgcaggtatg gaaatgacat atacttttgc ttttagaaga aaatcctgat | |
| 1741 | cagtgtgcac agaattcttc agggggcaag tgtgattcat tctgataaga tatagcatgg | |
| 1801 | cttagactgg gagactggca gaggctttga agatttcttt gctcaaattt tattcagcaa | |
| 1861 | gtatttacca tgcacctact atagcaggca acatttttag gaaatggtga atgttacaga | |
| 1921 | ggtgaataat acagcaagag tcgttgaaca tatggagttt atctattagt tggggagtga | |
| 1981 | atgttgacaa aggaataagt aaatacatag gcaagaaaga tacattacct gtgaaacagc | |
| 2041 | agcaggtaga ctgacagtgg agtatctaat acagcctatg gaagccagaa gatagtggga | |
| 2101 | tgacattttt ggagtactag tagaaatgtc atatgaagaa ctctgtagga atgtaacata | |
| 2161 | cggtcccata tatgaagctc ctgggtcaag tatacctgaa cataattcag ggatttgagg | |
| 2221 | gactttcttg taacctgagg atcaagatgt caaggaatta aaaacatgta taaaacattg | |
| 2281 | ttgtataaaa acccattaaa aagaatggaa gacactatag taaaatcatt gtgggtttag | |
| 2341 | ttgttataac acattttaaa aatctttgat cccaatcaat atttataaga aagaagaaat | |
| 2401 | atggaattat ttcctgagtc aaggagcagg gagagaatga ggaagaagag gaggaggagg | |
| 2461 | agggggagga ggagacaata aacctacttc ccaaagttaa caaacaaaaa gtgggaagag | |
| 2521 | gtcaaagact acaaggagta gaattaacgt caattgtttc tatgtttgag tctgaaaatt | |
| 2581 | ttttgtccct tctccaccaa cctatatatt gatacacata taaatgctaa aggcattttt | |
| 2641 | gaatttgaac agatcatttt ctttgtatgg ctgcctttaa aaaaaattca acctggtcac | |
| 2701 | tcttcctcaa catttactga ggtctaagtg ttcaatttag aacacatgct ttaataactc | |
| 2761 | agagacctgt catttgtcac aaatcttgcc tagagaaata ctcattagcg aattaggcag | |
| 2821 | aaagaggatg caaaataaaa aggcacagta gtcccctgat atccatggaa gactggttcc | |
| 2881 | aggacaccac caaacccctc cccgcaaata ccaaaatcca tggatgttca agtttcttaa | |
| 2941 | catatcatgg catagtattt gcatttaacc tacacacatc ctcttgtaca cttgaaatta | |
| 3001 | tctttagatt atttataata cttaatagaa tgtaaatgct atgtaactag ttgtgtatca | |
| 3061 | tttaggaaat gatcacaaga aaaaaagtct acagatgtta gtccagacac agccatcctt | |
| 3121 | tttttttttt tcaaatattt ttgatctgtg gttcattgca tccacagatg tggaacccat | |
| 3181 | ggatactgtg ggctaactgt attaataaaa aagtggaaac atcctaagtt tcatgggtgt | |
| 3241 | ttaaattggt cagcaacttc cttctgaaga agtatcagaa tttgtgagca atgttaatat | |
| 3301 | ttttgttttc tcactaagag ccacagttct gaatagaggt ttttaaaaag ccctagcaag | |
| 3361 | gtttctttag caatgaaact aacatttaac tgtatcatca gcttcgtgtt acatctcttt | |
| 3421 | cctgactgtt gggtgagccc tcctcggatg cttgcttctg gctacacgcc cctttaccct | |
| 3481 | tttctctgca ctgttttcat ctttataaag tcagagttgg tgtctatagg ctctctactg | |
| 3541 | ccacattcaa gacctgcctc gctcaatgtc accttcaaga tgcagaaata gggatttggg | |
| 3601 | aaggggattg tgaaattttc gaagtcttcc aaaatacttt gagaaactat atttggaagc | |
| 3661 | actttggggg gagaggttgg acaggaaggg tcttcagaga tcatcaaatt taactttcta | |
| 3721 | aatcctaagg aggaaaccga gactccagga tgtgaagtcc cttctctacc aaactagaat | |
| 3781 | ggatgcagga ggaatgtctg aggtgcaatc cttatccttt agcaaaggtg tcctctgcgt | |
| 3841 | cttctttaac ccatctcttg gacctccaga aagacagctg aggatggcaa ggggagtctg | |
| 3901 | gaaccactgg agtagccccc agcctcctcc ttggagggcc cccatgaagg aggcccttca | |
| 3961 | gtgacagaga ttgagagaga gggagggcga aaggaaggaa ggggagccag aggtgggagt | |
| 4021 | ggaagaggca gcctcgcctg gggctgattg gctcccgagg ccagggctct ccaagcggtt | |
| 4081 | tataagagtt ggggctgccg ggcgccctgc ccgctcgccc gcgcgcccca ggacccaaag | |
| 4141 | ccgggctcca agtcggcgcc ccacgtcgag gctccgccgc agcctccgga gttggccgca | |
| 4201 | gacaagaagg ggagggagcg ggagagggag gagagctccg aagcgagagg gccga | |

### Human Androgen Receptor Promoter Sequence

| | | |
|---|---|---|
| 1 | tctagaaaat aattcccaat attgaatccc aaagaattca acatttgggc tgtcgtttga | (SEQ ID NO. 6) |
| 61 | aagataagtt gaatttggtc atgaaggaag agagggggga tacaatttca gtaaaaggta | |
| 121 | acagcaaggt ccaaagacag tcaggtcttc agtagtatgg agtatattca gagggagcca | |
| 181 | agatgtctga tgtgaactaa aaagattggt ggttggtagg aggaagaggt gtgagaagag | |
| 241 | gctgtaaaga aaaattgaaa cttgattgtg atggacttta aaggctaggc tatgggactt | |
| 301 | ggacatgaat ctgcaggcca gtgtttgcag actggcgccc ataactgtct atcacagcaa | |
| 361 | cacagacatg tgttgtttgg cctgcagagg tttggcctgc atgatgattt taaaccatct | |
| 421 | gaattagtag ccatcatttt caaaaatcaa gagatgccac attaaaatat ggaatgctgc | |
| 481 | tgttcttgaa aataatgaaa catctggaac attgaggcca cattcctgac tgacagcaat | |
| 541 | cagttggagc tgcgtagtga ctgcccactt tacatggggc atctgatccc tagtcgatta | |
| 601 | cagctgccac cacttccctt tatctctcta ataccaagct cttttcactc atttttgtta | |
| 661 | cttaagagat atttgggttt gaaacctctg atgcaggtaa ttgagggtta tagagcagag | |
| 721 | gacagatgct atcagagttg tcttttaaga aagaaccctc tgttcttcat tttgttgaag | |
| 781 | atagcctgga agagggcagc caggggagaa gttagggctg gagctatgag aaagcataag | |
| 841 | atgagatgat ggcttcaaca ttgaggacag aaagaatatt gagatgagaa agtagtccat | |
| 901 | ataagcatct atgcaaagga aatagcagat gtcctcaaat cagcagaggc aacaactctg | |
| 961 | aaagtttatt cataagcccc tcttttcatc tccaatccag ttcaaatgta attatttaaa | |
| 1021 | ttgttcttca ctctccttcc tggatcatga atgagctcct taaatgcagg gtccacagtg | |
| 1081 | tcctattcat cagtgaattc caagtgccta gcacagagcc tggcaaatag taaatgctta | |
| 1141 | acaaatattc gttcagtgca tgaattggag tgattctcta ctttgcctca taagttgaaa | |
| 1201 | aaaggtttat tacataccta aatatgctga aatcacaggg catttggcaa ccccccaaaa | |
| 1261 | ccaaaactcc cagtttggaa acagaatttt aattctgtga aaataaaatc cattcattta | |
| 1321 | ttcaaaaaat atttattaaa caatgaccat gtccacacca ggctgagtcc taaggattca | |
| 1381 | atgatgaaca aaaaccaaca tgattcctgc tcttaggaaa catacagttc agtgaggaaa | |
| 1441 | acagattgtg agaagtcctc caacaaatac tgggtgctat taaaatatat taaaaggtga | |
| 1501 | gtgggtgagg gacttgagct agcctaggtg gttcaggaag tcttcctgga tgtgctgata | |
| 1561 | tgcataggca ttaactagat aaatagagag aaggatgaac caacattgca ggtagaggga | |
| 1621 | acagaatatg caaaggcagg aaggattatg gagtcgttgg aggacctgaa taaaggccca | |
| 1681 | gtgtaagtgg atctcagaaa acaggaggaa aggtgtatga gatgagatca gagaggcaga | |
| 1741 | tcatgtgggg tatggttaat gttttggact tttctattaa gagcaatggg gagacagtga | |
| 1801 | caggacttaa acggggaaat aatatgacca gattaaactt tctaaaaaac cctctatgca | |
| 1861 | aatatatatt gagagttaat tattgacaaa gattcaaagg caacaaagtg gagagagaat | |
| 1921 | agtattttca aaaaatggtg ccaaaacaat aggacatcta tattaaaagt tgggtatctg | |
| 1981 | tctacaaaac ttaattcaaa atggatcaca gacctaaatg taaaactgaa agctatacaa | |
| 2041 | cttctggaag gaaaacacag atgggaatct gtgtgatctt gagtttgaaa atgatttatt | |
| 2101 | atatctgaca ccataatccg taagttaaca taattcataa gtgaacaaag tgatgaactg | |
| 2161 | gacttcatca gaatttaaaa tgtttgtgct tcaaaagaca ctggtatgat aatgaagaca | |
| 2221 | aactacagat aagatattgt tgaatcatat ttctgataaa ggaattgtgg ctcagaatac | |
| 2281 | ataactctaa acccccataa taaattacaa gtagcccaat taaaaaaaaa aaaagagaaa | |
| 2341 | aaatttacag tcttcatcaa agaaagtatc aattgtaaaa taagcacatg aaaaatgctc | |
| 2401 | tgcatcttta ttcatggggg gatgaaataa aaattaaatg ggaaagacac ctctaattag | |
| 2461 | aatactaaaa ttaaaaagac tgaccatacc aagtattggt gaagtggaaa tgtaaaatga | |
| 2521 | tacaatcaac ttaggtagat gatttggaag tttcttacaa aagtaggtgt atacctaccc | |
| 2581 | tgtgactcac ccattccatg gctaagtatt tacctgagag aaatgaaaga atacatccat | |
| 2641 | acaaagatgt ttatacaaat atttatagca gttttatttg tagtagcccc aaactgaaaa | |
| 2701 | gaacccaaat gtccatcaaa agtgaatgga taaacaaagc gtggtacagc aatgcaatag | |
| 2761 | aatactactt agcaataaag aagaatgagc tagtgatata cataacagct taaatgtaca | |
| 2821 | tcaaaggcat tgtgctcagt gaaagatgca agtaaaaaaa aaaaagagta catgctgtat | |
| 2881 | agttccattg acataaaact ctggaaagtg aaaaacagtc tatactgaca gaaagcagat | |
| 2941 | cattggttgc ctgaggagga ggagtatagg agaggtggag ggaaaatgta caaagtggca | |
| 3001 | caataaaaac ttttggaatc atagatatat tcactatctt gattgagtga tgatttcatg | |
| 3061 | agtgcacgtg cgtgtgtcaa aaatgatcaa tttatgcaac tttaaatatg tgcagtttat | |
| 3121 | tgtatatatc aattatacct cagtacggct attaaaaaga aaccctctgg ctgcacaatg | |
| 3181 | cagaactgat tctaggaaag agtggaggga ggatgaccat ttacagtgct ccaggtggaa | |
| 3241 | gagaacggtg ccttctggaa gtgaactagg ttggcaacaa cagagatgaa ataaatgggc | |
| 3301 | agatgtgtga gatacttagg aaataaaacc cgatggtcac cattttccaa aggtcagctc | |
| 3361 | atcctggctt tccagagcaa agagctaggg aagactttat taataaatcc ctcttgaagt | |
| 3421 | tgcagaggaa gcttatagca gaaacttact ctcaacctga ctaatctgag agaacacctc | |
| 3481 | tggttccatt tgattactaa aaaactgcaa agaacaggag gagaaagaag aagaaagctg | |
| 3541 | gtacaaacag tgaacttata taatattaat caataattgt ctcttgttct taaaagcaat | |
| 3601 | gggaagaaaa tgagatttga gctggaagat cagagttcaa aatccaaata aagtatatgg | |
| 3661 | ccctaatatg cttatagtag ttaacctttc ctgataatga tataattgtt gacagcacca | |
| 3721 | tctttaaaat aaaataacat agtaatcctt cagatttgta gaagatcttt cctgtttaca | |
| 3781 | agtttgttct atacacatta tgtcttttaa atgacacact agccttctga gggtaactta | |
| 3841 | tattggcaac agttttcaga tgtggaaact gtgaagacaa tgttggtgat gtggaagcaa | |
| 3901 | cataaacttt ggagtctttc agacccaggt ttgaatgtca gactgctttt tattcagagt | |
| 3961 | aacttcagag cattatttct caccttaatt ttttttcagg cctctttgtg tctatgtgtc | |
| 4021 | ctcttcactc ctgtccattg tttcttcagt gatttttgcc accttccttc actgttagtg | |
| 4081 | tgtagacaca tagttctcct ggctctgaga gcctatgtta attccattct accatcctgc | |
| 4141 | cacggcccac tcaattccta ttgagcaatg ctagttgaaa gttgtggtgg gattaaatgt | |
| 4201 | tgcaatgagt attcaaatga ggttgaagta tctacgcatt ctacttacat atggtgaggt | |
| 4261 | atattcaagg aagctgtagc cattaaaatc tcaggaaata atttttcacc tcctcaggtg | |
| 4321 | aaagggtctt caggcctttg tgttctggaa ggttcattta tagccatttc ccaaatgaca | |
| 4381 | atgcgattga tgagtctaga gtctagctca aatagcaatg gactggaaga ctagtttagg | |
| 4441 | ttttactaat gtggaacata gaacaaatta tgtccttgtt tcagcctgtt catctgtgaa | |
| 4501 | atagagccta tcatatccag tcttccttgc ctttaggttt gagttacctt ctttggtcaa | |
| 4561 | ggtaagtaaa tgcctatgat gtttggctgt gcacaagata aagctacaac aaagctacaa | |
| 4621 | cccatctttt ctctgtagaa gactcaaaaa gcaaaagaga cccaggaaaa tctcggaatg | |
| 4681 | acttttggaa cagagagcct ccccagaatc agaagtcaag gaatttaaac atagggaagg | |
| 4741 | cccaggtctc tactgacata aaggaaagat gttttcttat aggtttcacg tttacatttt | |
| 4801 | ctctctcttg atcccattcc cacttgcatc tgccaccttt acacagggct tatgggacct | |
| 4861 | cctccacaaa agagcagttg cagtaaccca catcatcctc tacgccctgg ctgtccatca | |
| 4921 | agaggcgaaa agcagcccta tataggttct atccttggat agttccagtt gtaaagttta | |
| 4981 | aaatatgcga aggcaacttg gaaaagcaag cggctgcata caaagcaaac gtttacagag | |
| 5041 | ctctggacaa aattgagcgc ctatgtgtac atggcaagtg tttttagtgt ttgtgtgttt | |
| 5101 | acctgcttgt ctgggtgatt ttgcctttga gagtctggag agtagaagta ctggttaaag | |
| 5161 | gaacttccag acaggaagaa ggcagagaag agggtagaaa tgactctgat tcttggggct | |
| 5221 | gagggttcct agagcaaatg gcacaatgcc acgaggcccg atctatccct atgacggaat | |
| 5281 | ctaaggtttc agcaagtatc tgctggcttg gtcatggctt gctcctcagt ttgtaggaga | |
| 5341 | ctctcccact ctcccatctg cgcgctctta tcagtcctga aaagaacccc tggcagccag | |
| 5401 | gagcaggtat tcctatcgtc cttttcctcc ctccctcgcc ccaccctgtt ggttttttag | |
| 5461 | attgggcttt ggaaccaaat ttcctgagtg ctggcctcca ggaaatctgg agccctggcg | |
| 5521 | cctaaacctt ggtttaggaa accaggagct attcaggaag caggggtcct ccagggctag | |
| 5581 | agctagcctc tcctgccctc gcccacgctg cgccagcact tgtttctcca aagccactag | |
| 5641 | gcaggcgtta gcgcgcggtg aggggagggg agaaaaggaa aggggagggg agggaaaagg | |
| 5701 | aggtgggaag gcaaggaggc cggcccggtg ggggcgggac ccgactcgca aactgttgca | |
| 5761 | tttgctctcc acctcccagc gccccctccg agatcccggg gagccagctt gctgggagag | |
| 5821 | cgggacggtc cggagcaagc ccacaggcag aggaggcgac agagggaaaa agggccgagc | |
| 5881 | tagccgctcc agtgctgtac aggagccgaa gggacgcacc acgccagccc cagcccggct | |
| 5941 | ccagcgacag ccaacgcctc ttgcagcgcg gcggcttcga agccgccgcc cggagctgcc | |
| 6001 | ctttcctctt cggtgaagtt tttaaaagct gctaaagact cggaggaagc aaggaaagtg | |
| 6061 | cctggtagga ctgacggctg cctttgtcct cctcctctcc accccgcctc cccccaccct | |
| 6121 | gccttccccc cctcccccgt cttctctccc gcagctgcct cagtcggcta ctctcagcca | |
| 6181 | acccccctca ccacccttct ccccacccgc ccccccgccc ccgtcgccca gcgctgccag | |
| 6241 | cccgagtttg cagagaggta actccctttg gctgcgagcg ggcgagctag ctgcacattg | |
| 6301 | caaagaaggc tcttaggagc caggcgactg gggagcggct tcagcactgc agccacgacc | |
| 6361 | cgcctggtta ggctgcacgc ggagagaacc ctctgttttc ccccactctc tctccacctc | |
| 6421 | ctcctgcctt ccccaccccg agtgcggagc cagagatcaa aagatgaaaa ggcagtcagg | |
| 6481 | tcttcagtag ccaaaaaaca aaacaaacaa aaacaaaaaa caagaaataa aagaaaaaga | |
| 6541 | taataactca gttcttattt gcacctactt cagtggacac tgaatttgga aggtggagga | |
| 6601 | ttttgttttt ttcttttaag atctgggcat cttttgaatc tacccttcaa gtattaagag | |
| 6661 | acagactgtg agcctagcag ggcagatctt gtccaccgtg tgtcttcttc tgcacgagac | |
| 6721 | tttgaggctg tcagagcgct ttttgcgtgg ttgctcccgc aagtttcctt ctctggagct | |
| 6781 | tcccgcaggt gggcagctag ctgcagcgac taccgcatca tcacagcctg ttgaactctt | |
| 6841 | ctgagcaaga gaaggggagg cggggtaagg gaagtaggtg gaagattcag ccaagctcaa | |
| 6901 | ggatg | |

The invention further provides probes, primers and fragments of the osteomimicry regulatory region, and transcriptionally active fragments thereof. In one embodiment, purified nucleic acids consisting of at least 8 nucleotides (i.e., a hybridizable portion) of a regulatory region, and transcriptionally active fragments thereof gene sequence are provided; in other embodiments, the nucleic acids consist of at least 20 (contiguous) nucleotides, 25 nucleotides, 50 nucleotides, 100 nucleotides, 200 nucleotides, 500, 1000, 2000, 3000, 4000 or 5000 nucleotides of an osteomimicry regulatory region sequence, or transcriptionally active fragment thereof sequence. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

In another embodiment, the nucleic acids are smaller than 20, 25, 35, 200 or 500 nucleotides in length. Nucleic acids can be single or double stranded. The invention also encompasses nucleic acids hybridizable to or complementary to the foregoing sequences. In specific aspects, nucleic acids are provided which comprise a sequence complementary to at least 10, 20, 25, 50, 100, 200, 500 nucleotides or the entire osteomimicry regulatory region and transcriptionally active fragments gene.

The nucleotide sequences of the invention also include nucleotide sequences that have at least 65%, 70%, 75%, 80%, 85%, 90%, 95%, 98%, 99% or more nucleotide sequence identity to the nucleotide sequence depicted in SEQ ID NOs. 1, 2, 3, 4, 5, and 6, and/or transcriptionally active fragments thereof, which are capable of driving expression specifically within tumor and tissue cells with calcification potential.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions .times.100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12 and a gap penalty of 4 can be used. In an alternate embodiment, alignments can be obtained using the NA_MULTIPLE_ALIGNMENT 1.0 program, using a GapWeight of 5 and a GapLengthWeight of 1.

The percent identity between two sequences can be determined using techniques similar to those described above, with or without allowing gaps. In calculating percent identity, typically only exact matches are counted.

The invention also encompasses:

(a) DNA vectors that contain any of the foregoing osteomimicry regulatory sequences and/or their complements (i.e., antisense);

(b) DNA expression vectors that contain any of the foregoing osteomimicry regulatory element sequences operatively associated with a heterologous gene, such as a reporter gene; and

(c) genetically engineered host cells that contain any of the foregoing osteomimicry regulatory element sequences operatively associated with a heterologous gene such that the osteomimicry regulatory element directs the expression of the heterologous gene in the host cell.

Also encompassed within the scope of the invention are various transcriptionally active fragments of this regulatory region. A “transcriptionally active” or “transcriptionally functional” fragment of the osteomimicry regulatory region according to the present invention refers to a polynucleotide comprising a fragment of said polynucleotide which is functional as a regulatory region for expressing a recombinant polypeptide or a recombinant polynucleotide in a recombinant cell host. For the purpose of the invention, a nucleic acid or polynucleotide is “transcriptionally active” as a regulatory region for expressing a recombinant polypeptide or a recombinant polynucleotide if said regulatory polynucleotide contains nucleotide sequences which contain transcriptional information, and such sequences are operably associated to nucleotide sequences which encode the desired polypeptide or the desired polynucleotide.

In particular, the transcriptionally active fragments of the osteomimicry regulatory region of the present invention encompass those fragments that are of sufficient length to promote transcription of a heterologous gene, such as a reporter gene, when operatively linked to the osteomimicry regulatory sequence and transfected into tumor and tissue cells with calcification potential. Typically, the regulatory region is placed immediately 5′ to, and is operatively associated with the coding sequence. As used herein, the term “operatively associated” refers to the placement of the regulatory sequence immediately 5′ (upstream) of the reporter gene, such that transacting factors required for initiation of transcription, such as transcription factors, polymerase subunits and accessory proteins, can assemble at this region to allow RNA polymerase dependent transcription initiation of the reporter gene.

In one embodiment, the polynucleotide sequence chosen may further comprise other nucleotide sequences, either from the osteomimicry regulatory region, and transcriptionally active fragments thereof gene, or from a heterologous gene. In another embodiment, multiple copies of a promoter sequence, or a fragment thereof, may be linked to each other. For example, the promoter sequence, or a fragment thereof, may be linked to another copy of the promoter sequence, or another fragment thereof, in a head to tail, head to head, or tail to tail orientation. In another embodiment, an osteotropic-specific enhancer may be operatively linked to the osteomimicry regulatory sequence, or fragment thereof, and used to enhance transcription from the construct containing the osteomimicry regulatory sequence.

Also encompassed within the scope of the invention are modifications of the osteomimicry nucleotide sequences depicted in SEQ ID Nos. 1-6, respectively, without substantially affecting its transcriptional activities. Such modifications include additions, deletions and substitutions. In addition, any nucleotide sequence that selectively hybridizes to the complement of the sequence depicted in SEQ ID Nos. 1-6, respectively, under stringent conditions, and is capable of activating the expression of a coding sequence specifically within tumor and tissue cells with calcification potential is encompassed by the invention. Exemplary moderately stringent and high stringency hybridization conditions can be found in Ausubel F. M. et al., eds., 1989, Current Protocols in Molecular Biology, Vol. I, Green Publishing Associates, Inc., and John Wiley & sons, Inc., New York, at p. 2.10.3. Other conditions of high stringency which may be used are well known in the art.

The osteomimicry regulatory region, or transcriptionally functional fragments thereof, is preferably derived from a mammalian organism. Screening procedures which rely on nucleic acid hybridization make it possible to isolate gene sequences from various organisms. The isolated polynucleotide sequence disclosed herein, or fragments thereof, may be labeled and used to screen a cDNA library constructed from mRNA obtained from appropriate cells or tissues (e.g., calcified tissue) derived from the organism of interest. The hybridization conditions used should be of a lower stringency when the cDNA library is derived from an organism different from the type of organism from which the labeled sequence was derived. Further, mammalian osteomimicry regulatory region homologues may be isolated from, for example, bovine or other non-human nucleic acid, by performing polymerase chain reaction (PCR) amplification using two primer pools designed on the basis of the nucleotide sequence of the osteomimicry regulatory region disclosed herein. The template for the reaction may be cDNA obtained by reverse transcription of the mRNA prepared from, for example, bovine or other non-human cell lines, or tissue known to express the osteomimicry gene. For guidance regarding such conditions, see, e.g., Innis et al. (Eds.) 1995, PCR Strategies, Academic Press Inc., San Diego; and Erlich (ed) 1992, PCR Technology, Oxford University Press, New York, each of which is incorporated herein by reference in its entirety.

Promoter sequences within the 5′ non-coding regions of the osteomimicry gene may be further defined by constructing nested 5′ and/or 3′ deletions using conventional techniques such as exonuclease III or appropriate restriction endonuclease digestion. The resulting deletion fragments can be inserted into the promoter reporter vector to determine whether the deletion has reduced or obliterated promoter activity, such as described, for example, by Coles et al. (Hum. Mol. Genet., 7:791-800, 1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. These types of assays are well known to those skilled in the art.

The osteomimicry regulatory regions and transcriptionally functional fragments thereof, and the fragments and probes described herein which serve to identify osteomimicry regulatory regions and fragments thereof, may be produced by recombinant DNA technology using techniques well known in the art. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al, 1989, supra; also see the techniques described in “Oligonucleotide Synthesis”, 1984, Gait M. J. ed., IRL Press, Oxford, which is incorporated herein by reference in its entirety.

Alterations in the regulatory sequences can be generated using a variety of chemical and enzymatic methods which are well known to those skilled in the art. For example, regions of the sequences defined by restriction sites can be deleted. Oligonucleotide-directed mutagenesis can be employed to alter the sequence in a defined way and/or to introduce restriction sites in specific regions within the sequence. Additionally, deletion mutants can be generated using DNA nucleases such as Bal31, ExoIII, or S1 nuclease. Progressively larger deletions in the regulatory sequences are generated by incubating the DNA with nucleases for increased periods of time (see, e.g., Ausubel et al., 1989, supra).

The altered sequences are evaluated for their ability to direct expression of heterologous coding sequences in appropriate host cells. It is within the scope of the present invention that any altered regulatory sequences which retain their ability to direct expression of a coding sequence be incorporated into recombinant expression vectors for further use.

### Analysis of Osteomimecry Regulatory Region Activity

The osteomimicry regulatory region sequence, or transcriptionally active fragment thereof such as for example, and not by way of limitation, nucleotide sequences encoding the osteocalcin (OC), bone sialoprotein (BSP), SPARC/osteonectin (ON), osteopontin (OPN) and the receptor activator of NF-κB ligand (RANKL) regulatory region shows selective tissue and cell-type specificity; i.e., induces gene expression in osteotropic cells. Thus, the osteomimicry regulatory region sequence, and transcriptionally active fragments thereof, of the present invention may be used to induce expression of a heterologous coding sequence specifically in osteotropic cells. The activity and the specificity of the osteomimicry regulatory region sequence, and transcriptionally active fragments thereof can further be assessed by monitoring the expression level of a detectable polynucleotide operably associated with the osteomimicry regulatory region sequence, and transcriptionally active fragments thereof in different types of cells, tissues and cell lines engineered to contain the osteomimicry regulatory region sequence, and transcriptionally active fragments thereof. As discussed hereinbelow, the detectable polynucleotide may be either a polynucleotide that specifically hybridizes with a predefined oligonucleotide probe, or a polynucleotide encoding a detectable protein. The osteomimicry regulatory region sequence, and transcriptionally active fragments thereof can then be used to screen for candidate compounds or substances that interfere with the expression of the heterologous coding sequence. Such identified compounds or substances that interfere with osteomimicry regulatory region sequence, and transcriptionally active fragments thereof will be likely candidate compounds that interfere with the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL).

### Osteomimecry Regulatory Region Driven Reporter Constructs

The regulatory polynucleotides according to the invention may be advantageously part of a recombinant expression vector that may be used to express a coding sequence, or reporter gene, in a desired host cell or host organism. The osteomimicry regulatory region sequence, and transcriptionally active fragments thereof of the present invention, and transcriptionally active fragments thereof, may be used to direct the expression of a heterologous coding sequence. In particular, the present invention encompasses mammalian osteomimicry regulatory region sequence, and transcriptionally active fragments thereof. In accordance with the present invention, transcriptionally active fragments of the osteomimicry regulatory region sequence, and transcriptionally active fragments thereof encompass those fragments of the region which are of sufficient length to promote transcription of a reporter coding sequence to which the fragment is operatively linked.

A variety of reporter gene sequences well known to those of skill in the art can be utilized, including, but not limited to, genes encoding fluorescent proteins such as green fluorescent protein (GFP), enzymes (e.g. CAT, beta-galactosidase, luciferase) or antigenic markers. For convenience, enzymatic reporters and light-emitting reporters analyzed by colorometric or fluorometric assays are preferred for the screening assays of the invention.

In one embodiment, for example, a bioluminescent, chemiluminescent or fluorescent protein can be used as a light-emitting reporter in the invention. Types of light-emitting reporters, which do not require substrates or cofactors, include, but are not limited to the wild-type green fluorescent protein (GFP) of Victoria aequoria (Chalfie et al., 1994, Science 263:802-805), and modified GFPs (Heim et al., 1995, Nature 373:663-4; PCT publication WO 96/23810). Transcription and translation of this type of reporter gene leads to the accumulation of the fluorescent protein in test cells, which can be measured by a fluorimeter, or a flow cytometer, for example, by methods that are well known in the art (see, e.g., Lackowicz, 1983, Principles of Fluorescence Spectroscopy, Plenum Press, New York).

Another type of reporter gene that may be used are enzymes that require cofactor(s) to emit light, including but not limited to, Renilla luciferase. Other sources of luciferase also are well known in the art, including, but not limited to, the bacterial luciferase (luxAB gene product) of Vibrio harveyi (Karp, 1989, Biochim. Biophys. Acta 1007:84-90; Stewart et al. 1992, J. Gen. Microbiol, 138:1289-1300), and the luciferase from firefly, Photinus pyralis (De Wet et al. 1987, Mol. Cell. Biol. 7:725-737), which can be assayed by light production (Miyamoto et al., 1987, J. Bacteriol. 169:247-253; Loessner et al 1996, Environ. Microbiol. 62:1133-1140; and Schultz & Yarus, 1990, J. Bacteriol. 172:595-602).

Reporter genes that can be analyzed using colorimetric analysis include, but are not limited to, .beta.-galactosidase (Nolan et al. 1988, Proc. Natl. Acad. Sci. USA 85:260307), beta.-glucuronidase (Roberts et al. 1989, Curr. Genet. 15:177-180), luciferase (Miyamoto et al., 1987, J. Bacteriol. 169:247-253), or .beta.-lactamase. In one embodiment, the reporter gene sequence comprises a nucleotide sequence which encodes a LacZ gene product, Pgalactosidase. The enzyme is very stable and has a broad specificity so as to allow the use of different histochemical, chromogenic or fluorogenic substrates, such as, but not limited to, 5-bromo-4-chloro-3-indoyl-.beta.-D-galactoside (X-gal), lactose 2,3,5-triphenyl-2H-tetrazolium (lactose-tetrazolium) and fluorescein galactopyranoside (see Nolan et al., 1988, supra).

In another embodiment, the product of the E. coli .beta.-glucuronidase gene (GUS) can be used as a reporter gene (Roberts et al. 1989, Curr. Genet. 15:177-180). GUS activity can be detected by various histochemical and fluorogenic substrates, such as Xglucuronide (Xgluc) and 4-methylumbelliferyl glucuronide.

In addition to reporter gene sequences such as those described above, which provide convenient colorimetric responses, other reporter gene sequences, such as, for example, selectable reporter gene sequences, can routinely be employed. For example, the coding sequence for chloramphenicol acetyl transferase (CAT) can be utilized, leading to osteomimicry regulatory region sequence, and transcriptionally active fragments thereof-dependent expression of chloramphenicol resistant cell growth. The use of CAT and the advantages of a selectable reporter gene are well known to those skilled in the art (Eikmanns et al. 1991, Gene 102:93-98). Other selectable reporter gene sequences also can be utilized and include, but are not limited to, gene sequences encoding polypeptides which confer zeocin (Hegedus et al. 1998, Gene 207:241-249) or kanamycin resistance (Friedrich & Soriano, 1991, Genes. Dev. 5:1513-1523).

Other coding sequences, such as toxic gene products, potentially toxic gene products, and antiproliferation or cytostatic gene products, also can be used. In another embodiment, the detectable reporter polynucleotide may be either a polynucleotide that specifically hybridizes with a predefined oligonucleotide probe, or a polynucleotide encoding a detectable protein, including a BSP polypeptide or a fragment or a variant thereof. This type of assay is well known to those skilled in the art.

Osteomimecry regulatory region sequence, and transcriptionally active fragments thereof driven reporter constructs can be constructed according to standard recombinant DNA techniques (see, e.g., Methods in Enzymology, 1987, volume 154, Academic Press; Sambrook et al. 1989, Molecular Cloning—A Laboratory Manual, 2nd Edition, Cold Spring Harbor Press, New York; and Ausubel et al. Current Protocols in Molecular Biology, Greene Publishing Associates and Wiley Interscience, New York, each of which is incorporated herein by reference in its entirety).

Methods for assaying promoter activity are well-known to those skilled in the art (see, e.g., Sambrook et al., Molecular Cloning A Laboratory Manual, Cold Spring Harbor Laboratory, Cold Spring Harbor, N.Y., 1989). An example of a typical method that can be used involves a recombinant vector carrying a reporter gene and genomic sequences from the osteomimicry regulatory region sequence depicted in SEQ ID NOs. 1-6, respectively. Briefly, the expression of the reporter gene (for example, green fluorescent protein, luciferase, .beta.-galactosidase or chloramphenicol acetyl transferase) is detected when placed under the control of a biologically active polynucleotide fragment. Genomic sequences located upstream of the first exon of the gene may be cloned into any suitable promoter reporter vector. For example, a number of commercially available vectors can be engineered to insert the osteomimicry regulatory region sequence, and transcriptionally active fragments thereof of the invention for expression in mammalian host cells. Non-limiting examples of such vectors are pSEAPBasic, pSEAP-Enhancer, ppgal-Basic, p.beta.gal-Enhancer, or pEGFP-1 Promoter Reporter vectors (Clontech, Palo Alto, Calif.) or pGL2-basic or pGL3-basic promoterless luciferase reporter gene vector (Promega, Madison, Wis.). Each of these promoter reporter vectors include multiple cloning sites positioned upstream of a reporter gene encoding a readily assayable protein such as secreted alkaline phosphatase, green fluorescent protein, luciferase or beta.-galactosidase. The osteomimicry regulatory region sequence, and transcriptionally active fragments thereof are inserted into the cloning sites upstream of the reporter gene in both orientations and introduced into an appropriate host cell. The level of reporter protein is assayed and compared to the level obtained with a vector lacking an insert in the cloning site. The presence of an elevated expression level in the vector containing the insert with respect the control vector indicates the presence of a promoter in the insert.

Expression vectors that comprise a osteomimicry regulatory region sequence, and transcriptionally active fragments thereof may further contain a gene encoding a selectable marker. A number of selection systems may be used, including but not limited to, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026) and adenine phosphoribosyltransferase (Lowy et al., 1980, Cell 22:817) genes, which can be employed in tk.sup.-, hgprt.sup.- or aprt.sup.-cells, respectively. Also, antimetabolite resistance can be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes include trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthetase (Bebbington et al., 1992, Biotech 10:169).

### Characterization of Transcriptionally Active Osteomimecry Regulatory Region Sequences, and Transcriptionally Active Fragments Thereof

A fusion construct comprising an osteomimicry regulatory region sequence, and transcriptionally active fragments thereof, or a fragment thereof, can be assayed for transcriptional activity. As a first step in promoter analysis, the transcriptional start point (+1 site) of the osteotropic-specific gene under study has to be determined using primer extension assay and/or RNAase protection assay, following standard methods (Sambrook et al., 1989, Molecular Cloning: A Laboratory Manual, Cold Spring Harbor, Cold Spring Harbor Press). The DNA sequence upstream of the +1 site is generally considered as the promoter region responsible for gene regulation. However, downstream sequences, including sequences within introns, also may be involved in gene regulation. To begin testing for promoter activity, a −3 kb to +3 kb region (where +1 is the transcriptional start point) may be cloned upstream of the reporter gene coding region. Two or more additional reporter gene constructs also may be made which contain 5′ and/or 3′ truncated versions of the regulatory region to aid in identification of the region responsible for osteotropic-specific expression. The choice of the type of reporter gene is made based on the application.

In a preferred embodiment, a GFP reporter gene construct is used. The application of green fluorescent protein (GFP) as a reporter is particularly useful in the study of osteotropic-specific gene promoters. A major advantage of using GFP as a reporter lies in the fact that GFP can be detected in freshly isolated tumor and tissue cells with calcification potential without the need for substrates.

In another embodiment of the invention, a Lac Z reporter construct is used. The Lac Z gene product, .beta.-galactosidase, is extremely stable and has a broad specificity so as to allow the use of different histochemical, chromogenic or fluorogenic substrates, such as, but not limited to, 5-bromo-4-chloro-3-indoyl-.beta.-D-galactoside (X-gal), lactose 2,3,5-triphenyl-2H-tetrazolium (lactose-tetrazolium) and fluorescein galactopyranoside (see Nolan et al., 1988, supra).

For promoter analysis in transgenic mice, GFP that has been optimized for expression in mammalian cells is preferred. The promoterless cloning vector pEGFP1 (Clontech, Palo Alto, Calif.) encodes a red shifted variant of the wild-type GFP which has been optimized for brighter fluorescence and higher expression in mammalian cells (Cormack et al., 1996, Gene 173:33; Haas et al., 1996, Curr. Biol. 6:315). Moreover, since the maximal excitation peak of this enhanced GFP (EGFP) is at 488 nm, commonly used filter sets such as fluorescein isothiocyanate (FITC) optics which illuminate at 450-500 nm can be used to visualize GFP fluorescence. pEGFP1 proved to be useful as a reporter vector for promoter analysis in transgenic mice (Okabe et al, 1997, FEBS Lett. 407:313). In an alternate embodiment, transgenic mice containing transgenes with an osteomimicry regulatory region sequence, and transcriptionally active fragments thereof upstream of a luciferase reporter gene are utilized.

Putative osteomimicry regulatory region sequences, and transcriptionally active fragments thereof can be prepared (usually from a parent phage clone containing 8-10 kb genomic DNA including the promoter region) for cloning using methods known in the art. In one embodiment, for example, promoter fragments are cloned into the multiple cloning site of a luciferase reporter vector. In one embodiment, restriction endonucleases are used to excise the osteomimicry regulatory region sequence, and transcriptionally active fragments thereof to be inserted into the reporter vector. However, the feasibility of this method depends on the availability of proper restriction endonuclease sites in the regulatory fragment. In a preferred embodiment, the required promoter fragment is amplified by polymerase chain reaction (PCR; Saiki et al., 1988, Science 239:487) using oligonucleotide primers bearing the appropriate sites for restriction endonuclease cleavage. The sequence necessary for restriction cleavage is included at the 5′ end of the forward and reverse primers which flank the regulatory fragment to be amplified. After PCR amplification, the appropriate ends are generated by restriction digestion of the PCR product. The osteomimicry regulatory region sequence, and transcriptionally active fragments thereof, generated by either method, are then ligated into the multiple cloning site of the reporter vector following standard cloning procedures (Sambrook et al., 1989, supra). It is recommended that the DNA sequence of the PCR generated promoter fragments in the constructs be verified prior to generation of transgenic animals. The resulting reporter gene construct will contain the putative osteomimicry regulatory region sequence, and transcriptionally active fragments thereof located upstream of the reporter gene open reading frame, e.g., GFP or luciferase cDNA. The osteomimicry regulatory region sequence, and transcriptionally active fragments thereof with the reporter gene can then be used to screen for candidate compounds or substances that interfere with the expression of the heterologous coding sequence. Such identified compounds or substances that interfere with osteomimicry regulatory region sequence, and transcriptionally active fragments thereof will be likely candidate compounds that interfere with the ability of cancer cells to express highly restricted bone-like proteins comprising, inter alia, one or more of osteocalcin (OC), bone sialoprotein (BSP), SPARC/osteonectin (ON), osteopontin (OPN) and the receptor activator of NF-κB ligand (RANKL).

### Osteomimecry Regulatory Region Sequence Analysis Using Transgenic Mice

The mammalian osteomimicry regulatory region sequences, and transcriptionally active fragments thereof can be used to direct expression of, inter alia, a reporter coding sequence, a homologous gene or a heterologous gene in transgenic animals specifically within tumor and tissue cells with calcification potential. Animals of any species, including, but not limited to, mice, rats, rabbits, guinea pigs, pigs, micro-pigs, goats, sheep, and non-human primates, e.g., baboons, monkeys and chimpanzees may be used to generate transgenic animals. The term “transgenic,” as used herein, refers to non-human animals expressing osteomimicry regulatory region and transcriptionally active fragments thereof sequences from a different species (e.g., mice expressing human osteomimicry regulatory region and transcriptionally active fragments thereof sequences), as well as animals that have been genetically engineered to over-express endogenous (i.e., same species) osteomimicry regulatory region and transcriptionally active fragments thereof sequences or animals that have been genetically engineered to knock-out specific sequences.

In one embodiment, the present invention provides for transgenic animals that carry a transgene such as a reporter gene, therapeutic and/or toxic coding sequence under the control of the osteomimicry regulatory region and transcriptionally active fragments thereof, in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (1992, Proc. Natl. Acad. Sci. USA 89:6232-6236). When it is desired that the transgene be integrated into the chromosomal site of the endogenous corresponding gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene.

Any technique known in the art may be used to introduce a transgene under the control of the osteomimicry regulatory region and transcriptionally active fragments thereof into animals to produce the founder lines of transgenic animals. Such techniques include, but are not limited to, pronuclear microinjection (Hoppe & Wagner, 1989, U.S. Pat. No. 4,873,191); nuclear transfer into enucleated oocytes of nuclei from cultured embryonic, fetal or adult cells induced to quiescence (Campbell et al., 1996, Nature 380:64-66; Wilmut et al., Nature 385:810-813); retrovirus gene transfer into germ lines (Van der Putten et al., 1985, Proc. Natl. Acad. Sci., USA 82:6148-6152); gene targeting in embryonic stem cells (Thompson et al., 1989, Cell 65:313-321); electroporation of embryos (Lo, 1983, Mol. Cell. Biol. 31:1803-1814); and sperm-mediated gene transfer (Lavitrano et al., 1989, Cell 57:717-723; see, Gordon, 1989, Transgenic Animals, Intl. Rev. Cytol. 115:171-229).

For example, for microinjection of fertilized eggs, a linear DNA fragment (the transgene) containing the regulatory region, the reporter gene and the polyadenylation signals, is excised from the reporter gene construct. The transgene may be gel purified by methods known in the art, for example, by the electroelution method. Following electroelution of gel fragments, any traces of impurities are further removed by passing through Elutip D column (Schleicher & Schuell, Dassel, Germany).

In a preferred embodiment, the purified transgene fragment is microinjected into the male pronuclei of fertilized eggs obtained from B6 CBA females by standard methods (Hogan, 1986, Manipulating the Mouse Embryo, A Laboratory Manual. Cold Spring Harbor Laboratory Press, Cold Spring Harbor, N.Y.). Mice are analyzed transiently at several embryonic stages or by establishing founder lines that allow more detailed analysis of transgene expression throughout development and in adult animals. Transgene presence is analyzed by PCR using genomic DNA purified from placentas (transients) or tail clips (founders) according to the method of Vemet et al., Methods Enzymol. 1993; 225:434-451. Preferably, the PCR reaction is carried out in a volume of 100.mu.l containing 1.mu.g of genomic DNA, in 1.times. reaction buffer supplemented with 0.2 mM dNTPs, 2 mM MgCl.sub.2, 600.mu.M each of primer, and 2.5 units of Taq polymerase (Promega, Madison, Wis.). Each of the 30 PCR cycles consists of denaturation at 94.degree. C. for 1 min, annealing at 54.degree. C. for 1 min, and extension at 72.degree. C. for 1 min. The founder mice are then mated with C57B1 partners to generate transgenic F.sub.1 lines of mice.

Screening Assays for Compounds or Substances that Modulate Osteomimicry

Compounds or substances that interfere with the abnormal function and/or growth of tumor and tissue cells with calcification potential can provide therapies targeting defects in osteotropic-related disorders including, but not limited to, localized or disseminated osteosarcoma, lung, renal, colon, melanoma, thyroid, brain, multiple myeloma, breast and prostate cancers, and benign conditions, such as benign prostatic hyperplasia (BPH) or arterial sclerotic conditions where calcification occurs. Such compounds may be used to interfere with the onset or the progression of osteotropic-related disorders. Compounds or substances that stimulate or inhibit promoter activity also may be used to ameliorate symptoms of osteotropic-related disorders.

Genetically engineered cells, cell lines and/or transgenic animals containing a osteomimicry regulatory region and transcriptionally active fragments thereof, operably linked to a reporter gene, can be used as systems for the screening of agents that modulate osteomimicry regulatory region and transcriptionally active fragments thereof activity. Such transgenic mice provide an experimental model in vivo (or can be used as a source of primary cells or cell lines for use in vitro) which can be used to develop new methods of treating osteotropic-related disorders by targeting therapeutic agents to cause arrest in the progression of such disorders.

The present invention encompasses screening assays designed to identify compounds or substances that modulate activity of the osteomimicry regulatory region and transcriptionally active fragments thereof. The present invention encompasses in vitro and cell-based assays, as well as in vivo assays in transgenic animals. As described hereinbelow, compounds to be tested may include, but are not limited to, oligonucleotides, peptides, proteins, small organic or inorganic compounds, antibodies, etc.

Examples of compounds may include, but are not limited to, peptides, such as, for example, soluble peptides, including, but not limited to, Ig-tailed fusion peptides, and members of random peptide libraries; (see, e.g., Lam, et al, 1991, Nature 354:82-84; Houghten, et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72:767-778), antibodies (including, but not limited to, polyclonal, monoclonal, humanized, anti-idiotypic, chimeric or single chain antibodies, and FAb, F(ab′).sub.2 and FAb expression library fragments, and epitope-binding fragments thereof), and small organic or inorganic molecules.

Such compounds may further comprise compounds, in particular drugs or members of classes or families of drugs, known to ameliorate the symptoms of an osteotropic-related disorder.

Such compounds include, but are not limited to, families of antidepressants such as lithium salts, carbamazepine, valproic acid, lysergic acid diethylamide (LSD), pchlorophenylalanine, p-propyldopacetamide dithiocarbamate derivatives e.g., FLA 63; antianxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, .alpha.-methyl-tyrosine, reserpine, tetrabenazine, benzotropine, pargyline; noradrenergic agonists and antagonists e.g., clonidine, phenoxybenzamine, phentolamine, tropolone; nitrovasodilators (e.g., nitroglycerine, nitroprusside as well as NO synthase enzymes); and antagosists of growth factors (e.g., VEGF, FGF, angiopoetins and endostatin), androgen receptor antagonists, GPCR antagonists, PKA/CREB signal activation interrupters, b2m/PKA/CREB signaling interupters, CREB transcription factor and complex formation signal activation interrupters, or any combination thereof.

In one preferred embodiment, genetically engineered cells, cell lines or primary cultures of germ and/or somatic cells containing a mammalian osteomimicry regulatory region and transcriptionally active fragments thereof operatively linked to a heterologous gene are used to develop assay systems to screen for compounds which can inhibit sequence-specific DNA-protein interactions. Such methods comprise contacting a compound or substance to a cell that expresses a gene under the control of a osteomimicry regulatory region and transcriptionally active fragments thereof, measuring the level of the gene expression or gene product activity and comparing this level to the level of gene expression or gene product activity produced by the cell in the absence of the compound or substance, such that if the level obtained in the presence of the compound or substance differs from that obtained in its absence, a compound capable of modulating the expression of the mammalian osteomimicry regulatory region and transcriptionally active fragments thereof has been identified. Alterations in gene expression levels may be by any number of methods known to those of skill in the art e.g., by assaying for reporter gene activity, assaying cell lysates for mRNA transcripts, e.g. by Northern analysis or using other methods known in the art for assaying for gene products expressed by the cell.

In another embodiment, microdissection and transillumination can be used. These techniques offer a rapid assay for monitoring effects of putative drugs on osteotropic cells in transgenic animals containing a osteomimicry regulatory region and transcriptionally active fragments thereof-driven reporter gene. In this embodiment, a test agent is delivered to the transgenic animal by any of a variety of methods. Methods of introducing a test agent may include oral, intradermal, intramuscular, intraperitoneal, intravenous, subcutaneous, intranasal and via scarification (scratching through the top layers of skin, e.g., using a bifurcated needle) or any other standard routes of drug delivery. The effect of such test compounds on the osteotropic cells can be analyzed by the microdissection and transillumination of the osteoblastic cells. If the level of reporter gene expression observed or measured in the presence of the compound differs from that obtained in its absence, a compound capable of modulating the expression of the mammalian osteomimicry regulatory region and transcriptionally active fragments thereof has been identified.

In various embodiments of the invention, compounds that may be used in screens for modulators of osteotropic-related disorders include peptides, small molecules, both naturally occurring and/or synthetic (e.g., libraries of small molecules or peptides), cell-bound or soluble molecules, organic, non-protein molecules and recombinant molecules that may have osteomimicry regulatory region and transcriptionally active fragments thereof binding and/or interfering capacity and, therefore, may be candidates for pharmaceutical agents.

Alternatively, the proteins and compounds include endogenous cellular components which interact with osteomimicry regulatory region and transcriptionally active fragments thereof sequences in vivo. Cell lysates or tissue homogenates may be screened for proteins or other compounds which bind to the osteomimicry regulatory region and transcriptionally active fragments thereof. Such endogenous components may provide new targets for pharmaceutical and therapeutic interventions.

In one embodiment, libraries can be screened. Many libraries are known in the art that can be used, e.g., peptide libraries, chemically synthesized libraries, recombinant (e.g., phage display libraries), and in vitro translation-based libraries. In one embodiment of the present invention, peptide libraries may be used to screen for agonists or antagonists of osteomimicry regulatory region and transcriptionally active fragments thereof-linked reporter expression. Diversity libraries, such as random or combinatorial peptide or non-peptide libraries can be screened for molecules that specifically modulate osteomimicry regulatory region and transcriptionally active fragments thereof activity. Random peptide libraries consisting of all possible combinations of amino acids attached to a solid phase support may be used to identify peptides that are able to activate or inhibit osteomimicry regulatory region and transcriptionally active fragments thereof activities (Lam, K. S. et al., 1991, Nature 354:82-84). The screening of peptide libraries may have therapeutic value in the discovery of pharmaceutical agents that stimulate or inhibit the expression of osteomimicry regulatory region and transcriptionally active fragments thereof.

Examples of chemically synthesized libraries are described in Fodor et al., 1991, Science 251:767-773; Houghten et al., 1991, Nature 354:84-86; Lam et al., 1991, Nature 354:82-84; Medynski, 1994, BioTechnology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al, 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of example of non-peptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) also can be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

A specific embodiment of such an in vitro screening assay is described below. The osteomimicry regulatory region and transcriptionally active fragments thereof-reporter vector is used to generate transgenic mice from which primary cultures of osteomimicry regulatory region and transcriptionally active fragments thereof-reporter vector germ cells are established. About 10,000 cells per well are plated in 96-well plates in total volume of 100.mu.l, using medium appropriate for the cell line. Candidate inhibitors of the osteomimicry regulatory region and transcriptionally active fragments thereof are added to the cells. The effect of the inhibitors of the osteomimicry regulatory region and transcriptionally active fragments thereof can be determined by measuring the response of the reporter gene driven by the osteomimicry regulatory region and transcriptionally active fragments thereof. This assay could easily be set up in a high-throughput screening mode for evaluation of compound libraries in a 96-well format that reduce (or increase) reporter gene activity, but which are not cytotoxic. After 6 hours of incubation, 100.mu.l DMEM medium+2.5% fetal bovine serum (FBS) to 1.25% final serum concentration is added to the cells, which are incubated for a total of 24 hours (18 hours more). At 24 hours, the plates are washed with PBS, blot dried, and frozen at −80.degree. C. The plates are thawed the next day and analyzed for the presence of reporter activity.

In a preferred example of an in vivo screening assay, tumor or tissue cells with calcification potential derived from transgenic mice can be transplanted into mice with a normal or other desired phenotype (Brinster et al., 1994, Proc. Natl. Acad. Sci. USA 91:11298-302; Ogawa et al., 1997, Int. J. Dev. Biol. 41:111-12). Such mice can then be used to test the effect of compounds and other various factors on osteotropic-related disorders. In addition to the compounds and agents listed above, such mice can be used to assay factors or conditions that can be difficult to test using other methods, such as dietary effects, internal pH, temperature, etc.

Once a compound has been identified that inhibits or enhances osteomimicry regulatory region and transcriptionally active fragments thereof activity, it may then be tested in an animal-based assay to determine if the compound exhibits the ability to act as a drug to ameliorate and/or prevent symptoms of an osteotropic-related disorder, including, but not limited to, localized or disseminated osteosarcoma, lung, renal, colon, melanoma, thyroid, brain, multiple myeloma, breast and prostate cancers, and benign conditions, such as benign prostatic hyperplasia (BPH) or arterial sclerotic conditions where calcification occurs.

The assays of the present invention may be first optimized on a small scale (i.e., in test tubes), and then scaled up for high-throughput assays. The screening assays of the present invention may be performed in vitro, i.e., in test tubes, using purified components or cell lysates. The screening assays of the present invention may also be carried out in intact cells in culture and in animal models. In accordance with the present invention, test compounds which are shown to modulate the activity of the osteomimicry regulatory region and transcriptionally active fragments thereof in vitro, as described herein, will further be assayed in vivo in cultured cells and animal models to determine if the test compound has the similar effects in vivo and to determine the effects of the test compound on osteotropic-related disorders.

### Osteomimicry Modulatory Antisense, Ribozyme and Triple Helix Approaches

In another embodiment, the types of conditions, disorders, or diseases involving tumor and tissue cells with calcification potential which may be prevented, delayed, or rescued by modulating osteotropic-specific gene expression by using a osteomimicry regulatory region and/or transcriptionally active fragments thereof in conjunction with well-known antisense, gene “knock-out,” ribozyme and/or triple helix methods, are described. Such molecules may be designed to modulate, reduce or inhibit either unimpaired, or if appropriate, mutant osteotropic gene activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides which are complementary to an mRNA sequence. The antisense oligonucleotides will bind to the complementary mRNA sequence transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the sequence of interest could be used in an antisense approach to inhibit translation of endogenous mRNA. Antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit sequence expression. It is preferred that these studies utilize controls that distinguish between antisense gene inhibition and nonspecific biological effects of oligonucleotides. Additionally, it is envisioned that results obtained using the antisense oligonucleotide are compared with those obtained using a control oligonucleotide. It is preferred that the control oligonucleotide is of approximately the same length as the test oligonucleotide and that the nucleic acid of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86:6553-6556; Lemaitre, et al., 1987, Proc. Natl. Acad. Sci. U.S.A. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an .alpha.-anomeric oligonucleotide. An .alpha.-anomeric oligonucleotide forms specific double-stranded hybrids with complementary RNA in which, contrary to the usual beta.-units, the strands run parallel to each other (Gautier, et al., 1987, Nucl. Acids Res. 15:6625-6641). The oligonucleotide is a 2′-O-methylribonucleotide (Inoue, et al., 1987, Nucl. Acids Res. 15:6131-6148), or a chimeric RNA-DNA analogue (Inoue, et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16:3209), methylphosphonate oligonucleotides can be prepared by use of controlled pore glass polymer supports (Sarin, et al., 1988, Proc. Natl. Acad. Sci. U.S.A. 85:7448-7451), etc.

While antisense nucleotides complementary to an osteotropic-specific coding region sequence could be used, those complementary to the transcribed, untranslated region (for example, osteomimicry regulatory region and/or transcriptionally active fragments thereof) are most preferred.

Antisense molecules should be delivered to cells that express the osteotropic sequence in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies which specifically bind receptors or antigens expressed on the target cell surface) can be administered systemically.

A preferred approach to achieve intracellular concentrations of the antisense sufficient to suppress translation of endogenous mRNAs utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs which will form complementary base pairs with the endogenous sequence transcripts and thereby prevent translation of the mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′-long terminal repeat of Rous sarcoma virus (Yamamoto, et al., 1980, Cell 22:787-797), the herpes thymidine kinase promoter (Wagner, et al., 1981, Proc. Natl. Acad. Sci. U.S.A. 78:1441-1445), the regulatory sequences of the metallothionein gene (Brinster, et al., 1982, Nature 296:3942), etc. Any type of plasmid, cosmid, YAC or viral vector can be used to prepare the recombinant DNA construct that can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al, 1990, Science 247, 1222-1225).

Ribozymes are enzymatic RNA molecules capable of catalyzing the specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

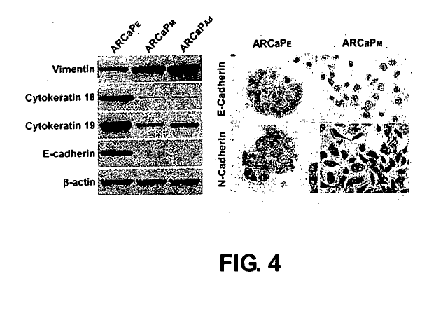

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions which form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334:585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al, 1985, Nature 317:230-234; Thomas and Capecchi, 1987, Cell 51:503-512; Thompson, et al., 1989, Cell 5:313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells which express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures which prevent transcription of the target gene in target cells in the body. (See generally, Helene, 1991, Anticancer Drug Des., 6(6):569-584; Helene, et al., 1992, Ann. N.Y. Acad. Sci., 660:27-36; and Maher, 1992, Bioassays 14(12):807-815).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleic acids may be pyrimidine-based, which will result in TAT and CGC+triplets across the three associated strands of the resulting triple helix. The pyrimidine-rich molecules provide base complementarity to a purine-rich region of a single strand of the duplex in a parallel orientation to that strand. In addition, nucleic acid molecules may be chosen which are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles which the possibility may arise wherein the concentration of normal target gene product present may be lower than is necessary for a normal phenotype. In such cases, to ensure that substantially normal levels of target gene activity are maintained, therefore, nucleic acid molecules which encode and express target gene polypeptides exhibiting normal target gene activity may be introduced into cells via gene therapy methods such as those described, below, which do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid-phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

### Gene Replacement Therapy

The nucleic acid sequences of the invention, described above, can be utilized for transferring recombinant nucleic acid sequences to cells and expressing said sequences in recipient cells. Such techniques can be used, for example, in marking cells or for the treatment of a disorder involving tumor or tissue cells with calcification potential. Such treatment can be in the form of gene replacement therapy. Specifically, one or more copies of a normal gene or a portion of the gene that directs the production of a gene product exhibiting normal gene function, may be inserted into the appropriate cells within a patient, using vectors that include, but are not limited to adenovirus, adeno-associated virus and retrovirus vectors, in addition to other particles that introduce DNA into cells, such as liposomes.

Methods for introducing genes for expression in mammalian cells are well known in the field. Generally, for such gene therapy methods, the nucleic acid is directly administered in vivo into a target cell or a transgenic mouse that expresses a osteomimetic-cancer specific regulatory region operably linked to a heterologous coding sequence. This can be accomplished by any method known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus or by administering it in linkage to a ligand subject to receptor-mediated endocytosis (see e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In one embodiment, techniques for delivery involve direct administration, e.g., by stereotactic delivery of such gene sequences to the site of the cells in which the gene sequences are to be expressed.

Additional methods that may be utilized to increase the overall level of gene expression and/or gene product activity include using targeted homologous recombination methods, as discussed above, to modify the expression characteristics of an endogenous gene in a cell or microorganism by inserting a heterologous DNA regulatory element such that the inserted regulatory element is operatively linked with the endogenous gene in question. Targeted homologous recombination can thus be used to activate transcription of an endogenous gene that is “transcriptionally silent”, i.e., is not normally expressed or is normally expressed at very low levels, or to enhance the expression of an endogenous gene that is normally expressed.

Further, the overall level of target gene expression and/or gene product activity may be increased by the introduction of appropriate target gene-expressing cells, preferably autologous cells, into a patient at positions and in numbers that are sufficient to ameliorate the symptoms of an osteotropic-related disorder. Such cells may be either recombinant or non-recombinant.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds or substances, such as those identified via techniques such as those described above that are capable of modulating activity of an osteomimicry regulatory region and transcriptionally active fragments thereof can be administered using standard techniques that are well known to those of skill in the art.

### Combination Therapies for Utilization and Targeting of Osteomimicry Using the Methods of the Invention

In each of the aforementioned aspects and embodiments of the invention, combination therapies other than those enumerated above are also specifically contemplated herein. In particular, the compositions of the present invention may be administered with one or more macrolide or non-macrolide antibiotics, anti-bacterial agents, anti-fungicides, anti-viral agents, and anti-parasitic agents, anti-inflammatory or immunomodulatory drugs or agents.

Examples of macrolide antibiotics that may be used in combination with the composition of the present invention include, inter alia, the following synthetic, semi-synthetic or naturally occurring microlidic antibiotic compounds: methymycin, neomethymycin, YC-17, litorin, erythromycin A to F, oleandomycin, roxithromycin, dirithromycin, flurithromycin, clarithromycin, davercin, azithromycin, josamycin, kitasamycin, spiramycin, midecamycin, rokitamycin, miokamycin, lankacidin, and the derivatives of these compounds. Thus, erythromycin and compounds derived from erythromycin belong to the general class of antibiotics known as “macrolides.” Examples of preferred erythromycin and erythromycin-like compounds include: erythromycin, clarithromycin, azithromycin, and troleandomycin.

Additional antibiotics, other than the macrolidic antibiotics described above, which are suitable for use in the methods of the present invention include, for example, any molecule that tends to prevent, inhibit or destroy life and as such, and as used herein, includes anti-bacterial agents, anti-fungicides, anti-viral agents, and anti-parasitic agents. These agents may be isolated from an organism that produces the agent or procured from a commercial source (e.g., pharmaceutical company, such as Eli Lilly, Indianapolis, Ind.; Sigma, St. Louis, Mo.). For example, the anti-TB antibiotic isoniazid (isonicotinic acid hydrazide), rifampin, ethambutol, ethionamide, streptomycin, amikacin, clofazimine, ofloxacin, levofloxacin, troveofloxacin, Pefloxacin, gatifloxacin, and moxifloxacin. Other examples of anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, oxazalidinones, and fluoroquinolones; and their various salts, acids, bases, and other derivatives.

Anti-fungal agents include, but are not limited to, caspofungin, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Anti-viral agents include, but are not limited to, valgancyclovir, amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.

Anti-parasitic agents include, but are not limited to, pirethrins/piperonyl butoxide, permethrin, iodoquinol, metronidazole, diethylcarbamazine citrate, piperazine, pyrantel pamoate, mebendazole, thiabendazole, praziquantel, albendazole, proguanil, quinidine gluconate injection, quinine sulfate, chloroquine phosphate, mefloquine hydrochloride, primaquine phosphate, atovaquone, co-trimoxazole (sulfamethoxazole/trimethoprim), and pentamidine isethionate.

In another aspect, in each of the aforementioned methods of the present invention, one may, for example, supplement the composition by administration of a therapeutically effective amount of one or more an anti-inflammatory or immunomodulatory drugs or agents. By “immunomodulatory drugs or agents”, it is meant, e.g., agents which act on the immune system, directly or indirectly, e.g., by stimulating or suppressing a cellular activity of a cell in the immune system, e.g., T-cells, B-cells, macrophages, or antigen presenting cells (APC), or by acting upon components outside the immune system which, in turn, stimulate, suppress, or modulate the immune system, e.g., hormones, receptor agonists or antagonists, and neurotransmitters; immunomodulators can be, e.g., immunosuppressants or immunostimulants. By “anti-inflammatory drugs”, it is meant, e.g., agents which treat inflammatory responses, i.e., a tissue reaction to injury, e.g., agents which treat the immune, vascular, or lymphatic systems.

Anti-inflammatory or immunomodulatory drugs or agents suitable for use in this invention include, but are not limited to, interferon derivatives, e.g., betaseron, beta.-interferon; prostane derivatives, e.g., compounds disclosed in PCT/DE93/0013, e.g., iloprost, cicaprost; glucocorticoid, e.g., cortisol, prednisolone, methylprednisolone, dexamethasone; immunsuppressives, e.g., cyclosporine A, FK-506, methoxsalene, thalidomide, sulfasalazine, azathioprine, methotrexate; lipoxygenase inhibitors, e.g., zileutone, MK-886, WY-50295, SC45662, SC-41661A, BI-L-357; leukotriene antagonists, e.g., compounds disclosed in DE 40091171 German patent application P 42 42 390.2; WO 9201675; SC-41930; SC-50605; SC-51146; LY 255283 (D. K. Herron et al., FASEB J. 2: Abstr. 4729, 1988); LY 223982 (D. M. Gapinski et al. J. Med. Chem. 33: 2798-2813, 1990); U-75302 and analogs, e.g., described by J. Morris et al., Tetrahedron Lett. 29: 143-146, 1988, C. E. Burgos et al., Tetrahedron Lett. 30: 5081-5084, 1989; B. M. Taylor et al., Prostaglandins 42: 211-224, 1991; compounds disclosed in U.S. Pat. No. 5,019,573; ONO-LB-457 and analogs, e.g., described by K. Kishikawa et al., Adv. Prostagl. Thombox. Leukotriene Res. 21: 407-410, 1990; M. Konno et al., Adv. Prostagl. Thrombox. Leukotriene Res. 21: 411-414, 1990; WF-11605 and analogs, e.g., disclosed in U.S. Pat. No. 4,963,583; compounds disclosed in WO 9118601, WO 9118879; WO 9118880, WO 9118883, antiinflammatory substances, e.g., NPC 16570, NPC 17923 described by L. Noronha-Blab. et al., Gastroenterology 102 (Suppl.): A 672, 1992; NPC 15669 and analogs described by R. M. Burch et al., Proc. Nat. Acad. Sci. USA 88: 355-359, 1991; S. Pou et al., Biochem. Pharmacol. 45: 2123-2127, 1993; peptide derivatives, e.g., ACTH and analogs; soluble TNF-receptors; TNF-antibodies; soluble receptors of interleukines, other cytokines, T-cell-proteins; antibodies against receptors of interleukins, other cytokines, and T-cell-proteins.

The therapeutic agents of the instant invention may be used for the treatment of animal subjects or patients, and more preferably, mammals, including humans, as well as mammals such as non-human primates, dogs, cats, horses, cows, pigs, guinea pigs, and rodent

### Pharmaceutical Preparations and Methods of Administration

The compounds or substances that are determined to modulate osteomimicry regulatory region and transcriptionally active fragments thereof activity or osteomimicry gene product activity can be administered to a patient at therapeutically effective doses to treat or ameliorate a disorder involving tumor or tissue cells with calcification potential. A therapeutically effective dose refers to that amount of the compound sufficient to result in amelioration of symptoms of such a disorder.

### Effective Dose

Toxicity and therapeutic efficacy of such compounds can be determined by standard pharmaceutical procedures in cell cultures or experimental animals, e.g., for determining the LD.sub.50 (the dose lethal to 50% of the population) and the ED.sub.50 (the dose therapeutically effective in 50% of the population). The dose ratio between toxic and therapeutic effects is the therapeutic index and it can be expressed as the ratio LD.sub.50/ED.sub.50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED.sub.50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC.sub.50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compounds may be combined with a carrier so that an effective dosage is delivered, based on the desired activity.

In addition to the formulations described previously, the compounds also may be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### EXAMPLES

The following specific examples are provided to better assist the reader in the various aspects of practicing the present invention. As these specific examples are merely illustrative, nothing in the following descriptions should be construed as limiting the invention in any way. Such limitations are, or course, defined solely by the accompanying claims.

### Example 1

### Prostate Cancer Metastasis: Role of the Host Microenvironment in Promoting Epithelial to Mesenchymal Transition and Increased Bone and Adrenal Gland Metastasis

BACKGROUND. The ARCaP cell line was established from the ascites fluid of a patient with metastatic prostate cancer. This study characterized the host microenvironmental role in cancer progression, epithelial to mesenchymal transition (EMT), and bone and adrenal metastasis in parental ARCaP and its derived cell subclones.

METHODS. Cytogenetic profiles, growth, migration, invasion, cellular interaction, drug sensitivities and gene expression of ARCaP cell subclones were compared. In vivo gene expression, behavior and metastasis of ARCaP subclones were analyzed by serial intracardiac injections into SCID mice.

RESULTS. ARCaP_{E} cells, with cobblestone morphology, underwent EMT through cellular interaction with host bone and adrenal gland. Lineage-derived ARCaP_{M} cells, with spindle-shape fibroblastic morphology, exhibited decreased cell adhesion and increased metastasis to bone and adrenal gland. Cytogenetic analyses of parental and ARCaP subclones confirmed their clonality.

CONCLUSIONS. ARCaP uniquely models the molecular basis of prostate cancer bone and adrenal metastases and epithelial to mesenchymal transition.

### Introduction

The diversity and heterogeneity of human prostate cancer cells is well appreciated. A broad spectrum of cancer cell behaviors include the ability to grow, invade surrounding normal tissues, and metastasize to distant organs (1-3). Despite similarities in the histologic presentation of prostate cancers at the time of disease diagnosis, their clinical behaviors, including time to disease progression and metastasis, sensitivity to hormones, chemotherapy and radiation, and propensity to relapse still cannot be predicted with certainty (4-7). Relevant models that could probe the phenotype, behavior and progression of cancer cells are lacking, as well as appropriate methods and sensitive biomarkers that can diagnose disease and reliably predict its clinical behavior early on. However, investigations have revealed a wealth of fresh information on the molecular basis of cancer metastasis through: 1) the development of useful transgenic (8-10) and xenograft (11-18) animal models and human prostate cancer cell lines (3); 2) characterization of the genetic diversity and heterogeneity of cancer cells and animal models; 3) the identification of specific loci that may harbor genes or clusters of genes contributing to the development of familial or sporadic forms of prostate cancer (19-21); and 4) elucidation of intracellular cell signaling and the roles of autocrine and paracrine factors in the tumor milieu that control the behavior of prostate cancer cells in interaction with the tumor microenvironment (2, 3, 22-24). Because prostate cancer has a predilection to metastasize to bone, resulting in increased patient mortality and morbidity, we sought to develop a highly metastatic prostate cancer model to evaluate the involvement of epithelial to mesenchymal transition (EMT) and the host microenvironment in prostate cancer bone and soft tissue metastases. This communication reports the cytogenetic, phenotypic and behavioral characterizations and gene expression profiles of parental ARCaP and ARCaP cell subclones subsequent to cellular interaction with mouse host cells in vivo.

### Materials and Methods

### Cell Culture

ARCaP cells were derived by our laboratory from the ascites fluid of a patient with metastatic carcinoma of the prostate (16). Cells were routinely maintained in a culture medium consisting of T medium (Life Technologies, Gaithersburg, Md.) and 5% fetal bovine serum (FBS) at 37° C. supplemented with 5% CO2. Limited dilution was performed by suspending 400 cells in 60 ml of T medium and seeding 100 μl per well in six 96-well plates. The wells containing one cell were expanded. Cell growth was determined by crystal violet assay (25). In brief, cells (3×10⁴ per well) were trypsinized and resuspended in T medium and seeded in 24-well plates under routine culture conditions. One plate of cells was removed at each designated time point and fixed with 0.5 ml of 1% glutaraldehyde for 15 min, stained with 0.5% crystal violet solution for 15 min, rinsed 4 times with water, air dried then eluted by Sorenson's solution for 30 min at room temperature. The optical density of the eluted solutions was read at 590 nm. The OD₅₉₀ was determined by an APECTRAmax 190 Microplate Reader and directly correlated with the number of cells (25). Conditioned media (CM) were collected from cells reaching 80% confluence, rinsed with PBS, replaced with serum-free T media and 2% TCM (Celox Laboratories Inc., St. Paul, Minn.) and cultured for 24 hours. The effects of CM on cell growth were determined in triplicate assays of three independent experiments with data expressed as average+/−SEM.

### Invasion and Migration Assays

A total of 35 μl of Matrigel Matrix (BD Biosciences, Bedford, Mass.; 100 μg/cm² surface area; diluted 1:5 in T medium) was placed on the inner upper Boyden chamber (BIOCOAT, 6.4 mm insert with 8 μm pores; Becton Dickinson Labware, Bedford, Mass.) and incubated for 30 min prior to adding to the cells. Cells (5×10⁴) were suspended in 500 μl of 0.1% BSA/T medium and added to the inner upper Boyden chamber. One ml of 0.1% BSA/T medium was added to the outer Boyden chamber. The chambers with or without Matrigel were placed in 24-well plates and incubated for 48 hrs. MTT solution (2.5 mg/ml; Sigma, St. Louis, Mo.) was added to both the inner (40 μL) and the outer (80 μl) chambers and incubated for an additional 4 hrs. The media were collected separately from each chamber, and cell-associated MTT crystals were scrubbed off with filter paper and dissolved separately in 500 μl DMSO (dimethyl sulfuroxide). The color intensity was measured at 590 nm against the appropriate blank controls (0.1% BSA/T medium with MTT solution and 500 μl DMSO). The % invasion was calculated by MTT eluted from cells invaded through the Boyden chamber/MTT eluted from cells that remained in the upper Boyden chamber plus those that invaded through the Boyden chamber. The % migration was conducted and calculated similarly to cell invasion, except the Boyden chambers were not coated with Matrigel (26,27). Relative invasion, migration and growth are presented as average+/−SEM of triplicate assays from two independent experiments.

In addition, migration was also determined by scratch wound assay (28) where cells (5×10⁵) were cultured in a 24-well plate. Then the 100% confluent cell layers were wounded with two parallel scratches using a sterile 200 μl pipette tip and rinsed with PBS. Images were taken at 0, 12, 24, 36 and 48 hr at the marked site using a ZEISS Axiovert 200M inverse light microscope (at 4×) and Openlab software (Improvision, Coventry, U. K.). Five measurements were taken from 0 to 48 hr. Mean widths were determined as a function of time with % migration tabulated as (Width 0 hr−Width at 12 to 48-hr)÷Width 0 hr×100%.

### Chemotherapeutic Sensitivity of Parental ARCaP and ARCaP Cell Subclones

Cells (5×10³ per well) were cultured in 96-well plates for 24 hr and then replaced with fresh cultured medium to which were added Paclitaxel, Etoposide, or Doxorubicin (Sigma, St. Louis, Mo.) at 4 different concentrations, followed by incubation for 96 hrs. Cell growth was measured using the MTT assay.

### Cytogenetic Analysis

Cells at 75% confluence in fresh media were exposed to Colcemid (20 ng/ml; Sigma) for 30 min at 37° C., rinsed two times with Hanks' balanced salt solution, and exposed to 0.01% trypsin for 5-7 min. The dislodged cells were neutralized with RPMI 1640 containing 10% FBS, and centrifuged at 1,700 rpm for 5 min. The cell pellet was disturbed and exposed to a hypotonic solution (0.06M KCl) for 20 min at room temperature. After centrifugation, the cells were fixed in acetic acid:methanol (1:3, v/v) for 15 min, rinsed three times with the fixative and stained with Giemsa solution for G-banding following routine procedures (16). Five to ten G-banded metaphase spreads were photographed for chromosome analyses for each cell clone.

### Protein Expression

Immunohistochemical (IHC) and western blot were used to determine the level of protein expression in cells. Monoclonal antibodies against cytokeratin 18/19 (CK18/19) were obtained from Santa Cruz Biotechnology, Inc. (Santa Cruz, Calif.); vimentin (VM) antibody from Dako Corp., Ltd. (Carpinteria, Calif.). Polyclonal antibodies to E-cadherin and N-cadherin were obtained from Santa Cruz. For immunohistochemical analysis, acetone (−20° C.) fixed cells or deparaffinized tissue sections (4μ) were treated with 3% hydrogen peroxide, blocked with Super Block (Scytek Laboratories, Logan, Utah), avidin and biotin (Vector Laboratories, Inc., Burlingame, Calif.) for 15 min each, and incubated with primary antibody overnight at 4° C. The signals were amplified by an avidin-biotin HRP system using multilink and label reagents (BioGenex) and hydrogen peroxide/DAB (3,3′-diaminobenzidine) as peroxidase substrate and chromogen (Sigma). Background activity was determined by 1) eliminating the primary antibody, 2) using matching mouse immunoglobulin subtypes or 3) normal goat or rabbit serum at appropriate dilutions. For Western Blot Analysis, cells were harvested at 80% confluence and rinsed twice with cold PBS. Cellular protein was extracted in a homogenization buffer (phosphate buffered saline with 1% Nonidet P-40, 0.5% sodium deoxycholate, 0.1% SDS, 10 mg/ml phenylmethylsulfonyl fluoride (PMSF), 1 mM sodium orthovanadate, 1 μg/ml leupeptin, and 1 μg/ml aprotinin). The total cell lysate (7.5-20 μg) was resolved by 7.5 or 10-20% SDS-polyacrylamide gel electrophoresis and transferred to a nitrocellulose membrane (NitroPure, Osmonics, Westborough, Mass.). The membrane was blocked for 1 hr at room temperature with 5% nonfat milk in TBST buffer (50 mM Tris-HCl, 150 mM NaCl, 0.05% Tween 20) and incubated with primary antibody in TBST blocking buffer for 1 hr at room temperature. The signal was detected by reacting with secondary antibody conjugated to horseradish peroxidase coupled with enhanced chemiluminescence (ECL) reagents (Amersham-Pharmacia Biotech, Piscataway, N.J.), and exposed on Hyperfilm (Amersham).

### Tumorigenicity and Metastasis In Vivo

Five to seven week old athymic ncr-nu/nu male mice (NC) were used as hosts. Cells at 80% confluence were changed with fresh T-medium the day before harvest. Cells were resuspended (2×10⁷/ml) and injected subcutaneously (1×10⁶ cells/100 μl/site, 4 sites per mouse). For intracardiac injection, cells were injected as 5×10⁵ cells/50 μl PBS/mouse using a 28G1/2 needle. Mice were anesthetized and placed in a supine position. The needle was inserted 5 mm above the middle of the left side of sternum. When fresh arterial blood appeared in the syringe this indicated the successful penetration into the left ventricle. Cells were infused slowly and directly into mouse left ventricle for systemic circulation. Tumor formation was monitored weekly and volume calculated as length×width×height×0.5236 (25,26). Metastases to distant organs were confirmed by radiography, necropsy and histomorphology of the tumor specimens.

Derivation of Cell Subclones from Tumor Tissues

Tumor tissue was freshly harvested, rinsed 3 times with PBS, replaced with cold PBS with antibiotics (Penicillin/Streptomycin (10,000 U/ml), placed on ice for 5 minutes, changed to cold T medium with 10% FBS and antibiotics and kept on ice. Tissue was cut into 0.5-1 mm³ pieces, put in cell culture dishes (separating at 0.5-1.0 cm), and briefly air dried to allow attachment. One to 2 drops of culture media were added on top of and around the tissue pieces to keep them humid and incubated. A, few more drops of media were added 6 hrs later followed by more media at 24 and 48 hrs. Tumor cells and mouse stromal cells started to emerge by 48 hrs with spindle-shape cells around the tissue and epithelial-like cells migrating away from the tissue piece, forming a rather “pure” colony by day 7-10. We used cloning disks (Scienceware, Pequannock, N.J.) to isolate pure cell subclones. Additional contaminating stromal cells were removed from epithelial cells by differential trypsinization (26).

### Results

ARCaP Subclones have Similar Cytogenetic Profiles But Distinct Morphology, Growth Rates, Gene Expression Profiles and Behaviors In Vitro

The ARCaP cells were originated from the ascites fluid of a patient with prostate cancer bony metastasis (16). The ARCaP cells harbor wild type androgen receptor (AR) and secrete low level of prostatic specific antigen (PSA) as compared to LNCaP cells. In contrast to LNCaP cells, parental ARCaP cells are invasive and cell growth is repressed by androgen both in vitro and in vivo. FIG. 1 shows five ARCaP cell subclones obtained by dilution cloning with marked differences in their morphology, ranging from cobblestone epithelial (IF11 or ARCaP_{E}) to spindle-shape mesenchymal cells (IA8 or ARCaP_{M}). Clones IID4 and IIC1 gave rise to morphologic features intermediate between ARCaP_{E} and ARCaP_{M}. One of the subclones, IF3, exhibited giant cell morphology with multinuclear features resembling matured osteoclasts. The growth rates of the 5 ARCaP cell subclones in vitro showed the mesenchyme-like ARCaP_{M} as the fastest, followed by IIC11, IID4 and IF3, with the epithelium-like ARCaP_{E} being the slowest (data not included).

### Cytogenetic Analyses

Cytogenetic analyses of parental ARCaP and the 5 cell subclones (Table 1) indicated that these cells are clonal. These subclones exhibited the same major marker chromosomes as ARCaP parental cells (16). However, each of the ARCaP cell subclones had its unique marker chromosomes. During the course of this study, both the morphology and the cytogenetic profiles of parental ARCaP and its subclones were stable despite repeated subculturing of the respective cells in vitro for more than 20 passages (unpublished results). The ARCaP cytogenetic profile (16) is distinct from the widely studied LNCaP cells (26). They do not share common marker chromosomes and can easily be identified and distinguished from each other based on their distinctive marker chromosomes. While the ARCaP subclones have distinct cytogenetic profiles, they also differ in their histomorphology, growth rate, migratory, invasive and metastatic potentials, and drug sensitivity (see Results). These properties are maintained in the mixed parental ARCaP cells by cell-cell interaction.

### Growth, Migration and Invasion of ARCaP_{E} and ARCaP_{M} Subclones In Vitro

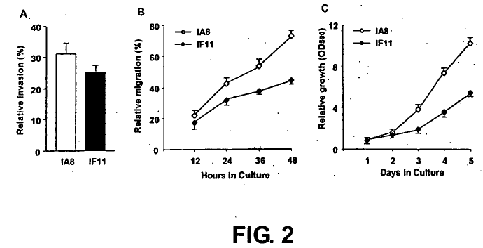

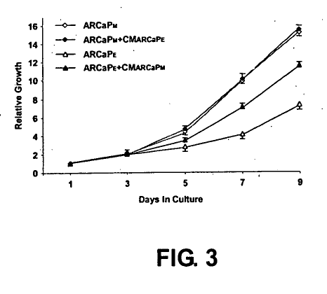

Since EMT has been associated with increased cancer cell invasion and migration (29-31), we evaluated the possible correlation between two morphologically distinct ARCaP subclones, cobblestone-shaped ARCaP_{E} and the spindle-shaped ARCaP_{M} subclones. Cell invasion using a Boyden Chamber coated with a Matrigel barrier (FIG. 2A), and migration as assessed by Scratch Wound Assay (FIG. 2B) correlated with cell growth rates (FIG. 2C), revealed higher migration and invasion by ARCaP_{M} than ARCaP_{E} cells (p<0.01). These 2 clones, after co-culturing (1:1) for more than 20 passages, still retained their original distinct morphology as seen in FIG. 1 without one clone being preferentially “selected” over the other (data not included). We hypothesize that clonal interaction occurs through factors secreted by one cell type exerting either a growth stimulatory or inhibitory effect on the other. To test this hypothesis, we replaced the cultured media of ARCaP_{E} with conditioned media (CM) collected from ARCaP_{M} and vice versa. FIG. 3 showed that CM from the fast-growing ARCaP_{M} cells stimulated the growth of the slow-growing ARCaP_{E} cells (p<0.01), but there was no growth inhibitory effect when the reverse experiment was conducted. These results suggest that a stimulatory rather than inhibitory factor plays a role in the maintenance of ARCaP_{E} and ARCaP_{M} subclones within the ARCaP cell population (see below).

### Gene Expression Profiles of ARCaP Subclones Grown in Culture

We conducted gene profile analysis of ARCaP subclones with specific emphasis on ARCaP_{E}, ARCaP_{M} and ARCaP-Ad (Adrenal). We found that, consistent with their morphologic features, ARCaP_{E} expressed dominantly epithelial markers while ARCaP_{M} and ARCaP-Ad expressed mesenchymal markers (FIG. 4), as evaluated by western blots (A) and IHC (B). These results were also confirmed by RT-PCR (data not included). Because of these morphologic and molecular characteristics thus the names ARCaP_{E}, ARCaP_{M} and ARCaP_{Ad} were given to IF11, IA8, and ARCaP-Adrenal subclones respectively. ARCaP_{E} expressed higher E-cadherin and cytokeratins 18 and 19 typically associated with epithelial cells, whereas ARCaP_{M} and ARCaP_{Ad} expressed more genes associated with mesenchymal cells, such as elevated vimentin and N-cadherin expression with concomitantly lower expression of epithelium-associated E-cadherin and cytokeratin genes. In addition to the classic EMT-associated genes, we also detected elevated protein expression of PSA, AR and PSMA and two new EMT-associated genes in ARCaP_{M} than that in ARCaP_{E} (data not included).

### Effects of Chemotherapeutic Agents on In Vitro Growth of ARCaP Cell Subclones

Because ARCaP represents a lethal form of human prostate cancer with the ability to invade and metastasize aggressively to bone and soft tissues, we sought to determine the in vitro sensitivities of ARCaP_{E} and ARCaP_{M} to several clinically used chemotherapeutic drugs and compared the results to invasive LNCaP lineage C4-2 cells treated with the same drugs. We found that ARCaP_{M} and ARCaP_{E} are more resistant to a DNA intercalating agent, doxorubicin (IC50s 5.5 and 3.4 uM for ARCaP_{M} and ARCaP_{E}, respectively) than C4-2 cells (IC50, 2.7 uM). ARCaP_{M} and ARCaP_{E} are also more resistant to topoisomerase inhibitor II, etoposide (IC50s 5.8 and 8.1 uM, respectively) than C4-2 cells (IC50, 5.6 uM). The relative resistance of ARCaP_{M} and ARCaP_{E}, compared to C4-2 cells, to the microtubule/tubulin assembly binding agent, paclitaxel, was also observed with IC50s at 39, 53, and 23.5 nM, respectively.

Comparison of the Tumorigenicity and Metastatic Potentials of ARCaP_{E} and ARCaP_{M} in Mice, and the Derivation of ARCaP_{M}-like Cells from Bone and Adrenal Gland Harvested from Animals Inoculated with ARCaP_{E} Cells

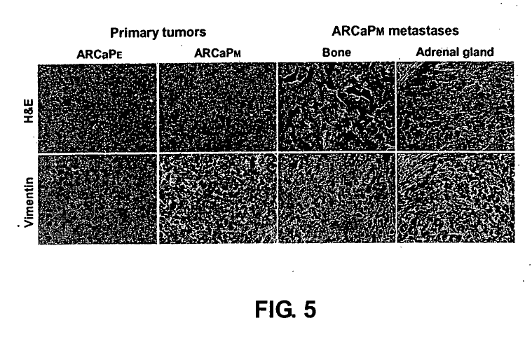

To confirm that differences in morphology, cell behavior, gene expression profiles, and sensitivity to chemotherapeutic drugs between ARCaP cell subclones in vitro reflect their tumorigenicity and metastatic potential in vivo, we conducted animal studies by inoculating two ARCaP cell subclones, ARCaP_{E} and ARCaP_{M}, into the left ventricles of immune-compromised SCID mice. The animals were observed closely and bone and soft tissue metastases were confirmed by x-ray, physical palpation and histomorphology. FIG. 5 showed the histopathology (top panels) and vimentin expression (IHC, bottom panels) of primary tumors from ARCaP_{E}, ARCaP_{M}, and metastatic lesions of bone and adrenal gland in mice inoculated intracardiacally with ARCaP cells. Similar to our experience in the orthotopic injection of parental ARCaP cells (16), tumor cells induced mixed osteoblastic and osteolytic responses in mice upon intracardiac injection of ARCaP subclones. Some mice also exhibited apparent cachexia and paraplegia at the later stage of bone metastasis (data not included).

The EMT-associated elevated expression of vimentin was demonstrated in ARCaP bone and adrenal meatastatic tumors as comparing with the primary tumor (FIG. 5). We derived ARCaP cell subclones from bone and adrenal gland metastases and further tested their metastatic potentials in mice. The incidence of bone metastasis ranged from 12.5% (1/8) for ARCaP_{E} cells, with a latency of 71 days, to 100% (9/9) for ARCaP_{M} cells, with a latency of 61 days (range 40-104 days). Interestingly, consistent with these observations, increased bone metastasis resulted from ARCaP cell interaction with mouse bone, through recycling of the injected ARCaP_{E} or ARCaP_{M} cells in the mouse hosts. Mice inoculated with ARCaP_{E} or ARCaP_{M} cells also developed increased adrenal gland metastasis, from 22% (4/18, latency 132 days, range 70-165 days) to 33% (3/9, latency 96 days, range 77 to 135 days). Remarkably, ARCaP_{Ad} metastasized only to host adrenal gland. We observed that both ARCaP_{Ad} and ARCaP_{M}-like cells derived from ARCaP_{E} had altered morphology and gene expression profiles (FIG. 4) resembled mesenchymal cells, suggesting that the bone and adrenal gland microenvironments had promoted EMT by facilitating the trans-differentiation of ARCaP_{E} cells toward ARCaP_{M} with preferential metastasis to bone or adrenal gland. In addition to adrenal gland, a low frequency of host mice also developed lymph node, liver and lung metastases (data not included).

### Discussion

We established an ARCaP human prostate cancer cell model to study the possible relationship between the host microenvironment, EMT, the critical transition of prostate cancer cells from epithelial to mesenchymal phenotype, (29-31), and the propensity of prostate cancer to metastasize to bone and soft tissue. We also correlated EMT with increased cell growth, migration, and invasion in vitro. EMT has been reported during embryonic development. The invasion front of the developing organ resembles that of the tumor, exhibiting increased cell motility, invasion and migration as observed in breast and bladder cancers. In the ARCaP human prostate cancer progression model, EMT can be promoted by cellular interaction between an ARCaP human prostate cancer cell subclone, ARCaP_{E}, and host bone or adrenal gland. The derivative ARCaP_{M} and ARCaP_{Ad} cells have the propensity to metastasize to bone and adrenal gland, respectively. Through further cellular interaction with host adrenal gland, we derived a secondary generation of ARCaP_{Ad} cells. We observed, remarkably, that second generation ARCaP_{Ad} cells had their ability to metastasize restricted only to the host adrenal gland. Because of the similarities in cell morphology, gene expression profiles and behavior of ARCaP_{M} derived from ARCaP_{E} through in vivo selection as a bone metastasis variant and the ARCaP_{M} IA8 subclone originally isolated from the ARCaP cells, we suggest that IA8 derived from IF11 through EMT transdifferentiation and the interaction of ARCaP_{E} with the host bone. Following cellular interaction between human prostate cancer ARCaP_{E} cells and the mouse host, we observed changes in morphology, gene expression and behavior in this cell clone to resemble a mesenchymal cell type, express mesenchymal genes, and show increased invasion and migration in vitro and metastasis to bone and adrenal gland in live mice (FIG. 2-5). The changes in gene expression profile, such as increased expression of vimentin and N-cadherin and decreased expression of E-cadherin and cytokeratin 18 and 19, are consistent with the morphologic switch of ARCaP cells by EMT, with increased metastatic potential, as reported in several other tumor types (32-35). We suggest that the host microenvironment plays an important role in facilitating EMT and subsequent prostate cancer metastasis to the skeleton and soft tissues (3). We observed that despite the clonal origin of ARCaP cells, they present as distinct morphologic and molecular variants with diverse ability to metastasize to bone and adrenal gland. Our results suggest that soluble stimulatory factor(s) secreted by prostate cancer cells may be responsible for the maintenance of tumor cell heterogeneity in ARCaP cells when cultured in vitro (FIG. 3). These observations are consistent with the published literature, where soluble factors such as TGFβ and/or EGF can confer EMT in cultured cells, resulting in altered cell growth and behaviors such as cell motility, invasion and metastasis in vitro (29, 31, 33, 35).

The fact that host interaction enhances EMT and promotes ARCaP cells to migrate, invade, and metastasize in this model suggests that clinical bone and adrenal gland metastases of prostate cancer cells may be acquired and facilitated by cellular interaction with host microenvironment. Based on the results of this and our previous studies (3, 15, 16, 26), it is likely that resident fibroblasts in the prostate, bone or adrenal gland or cells recruited from hosts, such as inflammatory and marrow stem cells (36-38), can instigate prostate cancer cells to gain increased malignant potential through the local production of soluble factors, reactive oxygen species and/or extracellular matrices that prompt the tumor cells for enhanced growth and metastasis (30, 35, 37, 38). Using marginally tumorigenic LNCaP cells as model, we showed previously that co-inoculating LNCaP cells with either non-tumorigenic human prostate stromal fibroblast or a human osteosarcoma cell line (25,39) formed large chimeric tumors. By cloning LNCaP cells from the chimeric tumors, we established lineage-derived LNCaP sublines C4-2 and C4-2B cells which, like other variants (25, 39, 40), exhibited increased lymph node and bone metastasis. Similar results, i.e. an increased propensity for local tumor formation and distant metastases, were obtained with ARCaP cells as described in the present communication and other human prostate cancer cell lines, whereby a human prostate cancer cell line when injected alone, without the presence of stromal fibroblasts, but with recruited host stromal cells, can promote prostate cancer progression (41-43). We posit that ARCaP interaction with bone or adrenal gland promotes irreversible EMT with subsequent increased invasive and migratory potential and the ability to metastasize to bone and soft tissues.

The demonstration that ARCaP cells undergo EMT in bone or adrenal gland and gain metastatic potential for various sites has several important clinical implications for controlling cancer growth and metastasis. First, the host microenvironment includes soluble and insoluble factors associated with or secreted by osteoblasts, osteoclasts, marrow stromal or stem cells that could play key roles promoting EMT, an important molecular transition by which cancer cells gain increased metastatic potential in response to the changing tumor microenvironment. These interactions could result in the promotion of cancer cell metastasis to soft tissues such as the adrenal gland, a documented site for human prostate cancer metastasis (44). Second, if EMT acquired by prostate cancer cells following cellular interaction with host bone or adrenal gland occurs in patients, this could be a potential target for prevention and treatment strategies. Third, since the host microenvironment was shown to promote EMT and prostate cancer progression, host-stroma-directed targeting of prostate cancer such as by the use of atrasentan (45), bisphosphonates (46), growth factor receptor antagonists (47), antiangiogenics (48) and radiopharmaceuticals (49), should be further explored to improve the treatment of cancer metastases.

### CONCLUSIONS

We demonstrated that the host microenvironment is a critical site for the transition of human prostate cancer cells from epithelial to mesenchymal morphology, resulting in increased metastatic potential for bone and adrenal gland. Clonal prostate cancer cells could have different histomorphologies, gene expression profiles, sensitivity toward cancer therapeutic drugs and variable behaviors in culture and in the host. We found that clonal interaction, possibly mediated by soluble factors secreted by prostate cancer cells, is responsible for maintaining tumor cell heterogeneity. Our study documented that EMT can be facilitated through cellular interaction between human prostate cancer cells and mouse skeleton or adrenal gland and that EMT could be exploited as a potential target for the prevention and treatment of human prostate cancer metastases.

### REFERENCES

1. Bavik C, Coleman I, Dean J P, Knudsen B, Plymate S, Nelson P S. The gene expression program of prostate fibroblast senescence modulates neoplastic epithelial cell proliferation through paracrine mechanisms. Cancer Res 2006; 66(2):794-802.
2. Barrett J M, Mangold K A, Jilling T, Kaul K L. Bi-directional interactions of prostate cancer cells and bone marrow endothelial cells in three-dimensional culture. Prostate 2005; 64(1):75-82.
3. Chung L W, Baseman A, Assikis V, Zhau H E. Molecular insights into prostate cancer progression: the missing link of tumor microenvironment. J Urol 2005; 173(1):10-20.
4. Bova G S, Parmigiani G, Epstein J I, Wheeler T, Mucci N R, Rubin M A. Web-based tissue microarray image data analysis: initial validation testing through prostate cancer Gleason grading. Hum Pathol 2001; 32(4):417-427.
5. DeMarzo A M, Nelson W G, Isaacs W B, Epstein J I. Pathological and molecular aspects of prostate cancer. Lancet 2003; 361(9361):955-964.
6. Carter H B, Piantadosi S, Isaacs J T. Clinical evidence for and implications of the multistep development of prostate cancer. J Urol 1990; 143(4):742-746.
7. De S, Chen J, Narizhneva N V, Heston W, Brainard J, Sage E H, Byzova T V. Molecular pathway for cancer metastasis to bone. J Biol Chem 2003; 278(40):39044-39050.
8. Kasper S, Sheppard P C, Yan Y, Pettigrew N, Borowsky A D, Prins G S, Dodd J G, Duckworth M L, Matusik R J. Development, progression, and androgen-dependence of prostate tumors in probasin-large T antigen transgenic mice: a model for prostate cancer. Lab Invest 1998; 78(6):i-xv.
9. Greenberg N M. Androgens and growth factors in prostate cancer: a transgenic perspective. Prostate Cancer Prostatic Dis 2000; 3(4):224-228.
10. Garabedian E M, Humphrey P A, Gordon J I. A transgenic mouse model of metastatic prostate cancer originating from neuroendocrine cells. Proc Natl Acad Sci USA 1998; 95(26):15382-15387.
11. Yonou H, Yokose T, Kamijo T, Kanomata N, Hasebe T, Nagai K, Hatano T, Ogawa Y, Ochiai A. Establishment of a novel species- and tissue-specific metastasis model of human prostate cancer in humanized non-obese diabetic/severe combined immunodeficient mice engrafted with human adult lung and bone. Cancer Res 2001; 61(5):2177-2182.
12. Wang Y, Xue H, Cutz J C, Bayani J, Mawji N R, Chen W G, Goetz L J, Hayward S W, Sadar M D, Gilks C B, Gout P W, Squire J A, Cunha G R, Wang Y Z. An orthotopic metastatic prostate cancer model in SCID mice via grafting of a transplantable human prostate tumor line. Lab Invest 2005; 85(11):1392-1404.
13. Nemeth J A, Harb J F, Barroso U, Jr., He Z, Grignon D J, Cher M L. Severe combined immunodeficient-hu model of human prostate cancer metastasis to human bone. Cancer Res 1999; 59(8):1987-1993.
14. Navone N M, Olive M, Ozen M, Davis R, Troncoso P, Tu S M, Johnston D, Pollack A, Pathak S, von Eschenbach A C, Logothetis C J. Establishment of two human prostate cancer cell lines derived from a single bone metastasis. Clin Cancer Res 1997; 3(12 Pt 1):2493-2500.
15. Wu T T, Sikes R A, Cui Q, Thalmann G N, Kao C, Murphy C F, Yang H, Zhau H E, Balian G, Chung L W. Establishing human prostate cancer cell xenografts in bone: induction of osteoblastic reaction by prostate-specific antigen-producing tumors in athymic and SCID/bg mice using LNCaP and lineage-derived metastatic sublines. Int J Cancer 1998; 77(6):887-894.
16. Zhau H Y, Chang S M, Chen B Q, Wang Y, Zhang H, Kao C, Sang Q A, Pathak S J, Chung L W. Androgen-repressed phenotype in human prostate cancer. Proc Natl Acad Sci USA 1996; 93(26):15152-15157.
17. Wainstein M A, He F, Robinson D, Kung H J, Schwartz S, Giaconia J M, Edgehouse N L, Pretlow T P, Bodner D R, Kursh E D, et al. CWR22: androgen-dependent xenograft model derived from a primary human prostatic carcinoma. Cancer Res 1994; 54(23):6049-6052.
18. Corey E, Quinn J E, Buhler K R, Nelson P S, Macoska J A, True L D, Vessella R L. LuCaP 35: a new model of prostate cancer progression to androgen independence. Prostate 2003; 55(4):239-246.
19. Carter B S, Beaty T H, Steinberg G D, Childs B, Walsh P C. Mendelian inheritance of familial prostate cancer. Proc Natl Acad Sci USA 1992; 89(8):3367-3371.
20. Visakorpi T, Kallioniemi A H, Syvanen A C, Hyytinen E R, Karhu R, Tammela T, Isola J J, Kallioniemi O P. Genetic changes in primary and recurrent prostate cancer by comparative genomic hybridization. Cancer Res 1995; 55(2):342-347.
21. Gronberg H, Damber L, Damber J E. Studies of genetic factors in prostate cancer in a twin population. J Urol 1994; 152(5 Pt 1):1484-1487; discussion 1487-1489.
22. Sikes R A, Nicholson B E, Koeneman K S, Edlund N M, Bissonette E A, Bradley M J, Thalmann G N, Cecchini M G, Pienta K J, Chung L W. Cellular interactions in the tropism of prostate cancer to bone. Int J Cancer 2004; 110(4):497-503.
23. Huang H, Muddiman D C, Tindall D J. Androgens negatively regulate forkhead transcription factor FKHR (FOXO1) through a proteolytic mechanism in prostate cancer cells. J Biol Chem 2004; 279(14):13866-13877.
24. Wu Y M, Robinson D R, Kung H J. Signal pathways in up-regulation of chemokines by tyrosine kinase MER/NYK in prostate cancer cells. Cancer Res 2004; 64(20):7311-7320.
25. Gleave M, Hsieh J T, Gao C A, von Eschenbach A C, Chung L W. Acceleration of human prostate cancer growth in vivo by factors produced by prostate and bone fibroblasts. Cancer Res 1991; 51(14):3753-3761.
26. Thalmann G N, Sikes R A, Wu T T, Degeorges A, Chang S M, Ozen M, Pathak S, Chung L W. LNCaP progression model of human prostate cancer: androgen-independence and osseous metastasis. Prostate 2000; 44(2):91-103 July 101; 144(102).
27. Hoosein N M, Logothetis C J, Chung L W. Differential effects of peptide hormones bombesin, vasoactive intestinal polypeptide and somatostatin analog RC-160 on the invasive capacity of human prostatic carcinoma cells. J Urol 1993; 149(5):1209-1213.
28. Kariya Y, Miyazaki K. The basement membrane protein laminin-5 acts as a soluble cell motility factor. Exp Cell Res 2004; 297(2):508-520.
29. Huber M A, Kraut N, Beug H. Molecular requirements for epithelial-mesenchymal transition during tumor progression. Curr Opin Cell Biol 2005; 17(5):548-558.
30. Thiery J P. Epithelial-mesenchymal transitions in development and pathologies. Curr Opin Cell Biol 2003; 15(6):740-746.
31. Larue L, Bellacosa A. Epithelial-mesenchymal transition in development and cancer: role of phosphatidylinositol 3′ kinase/AKT pathways. Oncogene 2005; 24(50):7443-7454.
32. Cattan N, Rochet N, Mazeau C, Zanghellini E, Mari B, Chauzy C, Stora de Novion H, Amiel J, Lagrange J L, Rossi B, Gioanni J. Establishment of two new human bladder carcinoma cell lines, CAL 29 and CAL 185. Comparative study of cell scattering and epithelial to mesenchyme transition induced by growth factors. Br J Cancer 2001; 85(9):1412-1417.
33. Nawshad A, Lagamba D, Polad A, Hay E D. Transforming growth factor-beta signaling during epithelial-mesenchymal transformation: implications for embryogenesis and tumor metastasis. Cells Tissues Organs 2005; 179(1-2):11-23.
34. Han G, Lu S L, Li A G, He W, Corless C L, Kulesz-Martin M, Wang X J. Distinct mechanisms of TGF-beta1-mediated epithelial-to-mesenchymal transition and metastasis during skin carcinogenesis. J Clin Invest 2005; 115(7):1714-1723.
35. Kang Y, Massaque J. Epithelial-mesenchymal transitions: twist in development and metastasis. Cell 2004; 118(3):277-279.
36. Aggarwal B B. Nuclear factor-kappaB: the enemy within. Cancer Cell 2004; 6(3):203-208.
37. Kaplan R N, Riba R D, Zacharoulis S, Bramley A H, Vincent L, Costa C, MacDonald D D, Jin D K, Shido K, Kerns S A, Zhu Z, Hicklin D, Wu Y, Port J L, Altorki N, Port E R, Ruggero D, Shmelkov S V, Jensen K K, Rafli S, Lyden D. VEGFR1-positive haematopoietic bone marrow progenitors initiate the pre-metastatic niche. Nature 2005; 438(7069):820-827.
38. Reya T, Clevers H. Wnt signalling in stem cells and cancer. Nature 2005; 434(7035):843-850.
39. Thalmann G N, Anezinis P E, Chang S M, Zhau H E, Kim E E, Hopwood V L, Pathak S, von Eschenbach A C, Chung L W. Androgen-independent cancer progression and bone metastasis in the LNCaP model of human prostate cancer. Cancer Res 1994; 54(10):2577-2581.
40. Rhee H W, Zhau H E, Pathak S, Multani A S, Pennanen S, Visakorpi T, Chung L W. Permanent phenotypic and genotypic changes of prostate cancer cells cultured in a three-dimensional rotating-wall vessel. In Vitro Cell Dev Biol Anim 2001; 37(3):127-140.
41. Sramkoski R M, Pretlow T G, 2nd, Giaconia J M, Pretlow T P, Schwartz S, Sy M S, Marengo S R, Rhim J S, Zhang D, Jacobberger J W. A new human prostate carcinoma cell line, 22Rv1. In Vitro Cell Dev Biol Anim 1999; 35(7):403-409.
42. Pettaway C A, Pathak S, Greene G, Ramirez E, Wilson M R, Killion J J, Fidler I J. Selection of highly metastatic variants of different human prostatic carcinomas using orthotopic implantation in nude mice. Clin Cancer Res 1996; 2(9):1627-1636.
43. Klein K A, Reiter R E, Redula J, Moradi H, Zhu X L, Brothman A R, Lamb D J, Marcelli M, Belldegrun A, Witte O N, Sawyers C L. Progression of metastatic human prostate cancer to androgen independence in immunodeficient SCID mice. Nat Med 1997; 3(4):402-408.
44. Bates A W, Baithun S I. Secondary solid neoplasms of the prostate: a clinico-pathological series of 51 cases. Virchows Arch 2002; 440(4):392-396.
45. Nelson J B, Nabulsi A A, Vogelzang N J, Breul J, Zonnenberg B A, Daliani D D, Schulman C C, Carducci M A. Suppression of prostate cancer induced bone remodeling by the endothelin receptor A antagonist atrasentan. J Urol 2003; 169(3): 1143-1149.
46. Smith M R. Zoledronic acid to prevent skeletal complications in cancer: corroborating the evidence. Cancer Treat Rev 2005; 31 Suppl 3:19-25.
47. Wu J D, Odman A, Higgins L M, Haugk K, Vessella R, Ludwig D L, Plymate S R. In vivo effects of the human type I insulin-like growth factor receptor antibody A12 on androgen-dependent and androgen-independent xenograft human prostate tumors. Clin Cancer Res 2005; 11 (8):3065-3074.
48. Longoria R L, Cox M C, Figg W D. Antiangiogenesis: a possible treatment option for prostate cancer? Clin Genitourin Cancer 2005; 4(3):197-202.
49. Tu S M, Millikan R E, Mengistu B, Delpassand E S, Amato R J, Pagliaro L C, Daliani D, Papandreou C N, Smith T L, Kim J, Podoloff D A, Logothetis C J. Bone-targeted therapy for advanced androgen-independent carcinoma of the prostate: a randomised phase II trial. Lancet 2001; 357(9253):336-341.

TABLE I
Cytogenetic profiles of parental ARCaP and its 5 cell subclones.

| Cells | 1p+ | 1q+ | del5q | 5p+ | 6p+ | del8p | i(9q) | 12q+ | 15p+ | 18q+ | 21p+ | delX | t(13:15) | 8q+ | i(5q) | 6q+ |
|---|---|---|---|---|---|---|---|---|---|---|---|---|---|---|---|---|
| IIC11 | + | + | + | + | + | + | + | + | + | + | + | + | − | − | − | − |
| ARCaPM | + | + | + | + | + | + | + | + | +^{Δ} | + | + | + | +*^{Δ} | −^{Δ} | − | − |
| ARCaPE | + | + | + | + | + | + | + | + | −^{Δ} | + | + | + | −^{Δ} | +*^{Δ} | − | − |
| IID4 | + | + | + | + | + | − | + | + | + | + | + | + | − | − | +* | − |
| IF3 | + | − | + | + | + | + | + | + | + | + | + | + | − | − | − | +* |
| ARCaP | + | − | + | + | + | + | − | + | − | + | − | + | − | − | − | − |

^{Δ}Difference between ARCaP_{M} and ARCaP_{E}.
*Difference among the five clones.

Throughout this application various publications and patents are referenced. The disclosures of these publications and patents in their entireties are hereby incorporated by reference into this application in order to more fully describe the state of the art to which this invention pertains.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

TABLE 1

| | |
|---|---|
| b2-Adrenergic receptor | STAT1 |
| VEGF | b-Catenin |
| STAT3 | G protein-coupled receptor 56 |
| Glutathione peroxisase, | IGFBP3 |
| PDGF b peptide | IGF2R |
| ADAM17 | Heat shock 70 kDa protein 4 |
| IL-8 receptor b | ADAM15 |
| b2M, IGF2 | Vimentin |
| PSA | IGFBP2 |
| Tumor protein D52 | IGF1 |
| CREB-like2 | Phosphodiesterase 3A. |

### APPENDIX A

| b2M Target Genes | | b2M Target Genes | |
|---|---|---|---|
| (increased) | Function | (decreased) | Function |
| b2-Adrenergic | cell growth | IGF2R | cell growth |
| receptor | | | |
| VEGF | cell growth, cell | Heat shock 70 kDa | cell adhsion |
| | cycle | protein 4 | |
| STAT3 | cell mobility | ADAM15 | |
| Glutathione peroxiase | oxidative stress | Vimentin | EMT marker |
| PDGF b peptide | | IGFBP2 | cell growth |
| ADAM17 | | IGF1 | cell growth |
| IL-8 receptor b | cell growth, mobility | Phosphodiesterase 3A | |
| b2M | cell growth, | IGF2 | cell growth |
| | survival | | |
| PSA | prostate cancer | | |
| | progression | | |
| Tumor protein D52 | | | |
| CREB-like2 | cell growth, | | |
| | survival | | |
| STAT1 | apoptosis | | |
| b-Catenin | cell adhesion | | |
| G protein-coupled | cell growth, | | |
| receptor 56 | survival | | |
| IGFBP3 | cell growth | | |

### APPENDIX B

### A Partial List of GPCRs Antagonists in Cancer

| GPCR | antagonist |
|---|---|
| α1-adrenoceptor | Doxazosin, terazosin, |
| | tamsulosin |
| β₂-Adrenergic Receptor | ICI 118,551 |
| Bradykinin receptor | B-9870, BKM-570, etc |
| CXCR4 | AMD 3100 |
| Endothelin A Receptor (ETA) | ZD4054 (AstraZeneca) |
| Gonadotropin-releasing-hormone- | Abarelix, Antarelix, |
| receptor | Cetrorelix, Ganirelix |
| | acetate, Lturelix |
| Lysophosphatidic acid receptor | Ki16425 |
| Leukotriene B4 receptor | LY293111 |
| Platelet activation factor receptor | Y-24180 |
| Prostaglandin E(2) | indomethacin |
| Broad-spectrum G protein-coupled | Substance P analogues |
| receptor (GPCR) antagonists | |

### Antiandrogens

### Steroidal Androgens

| Name | Reference |
|---|---|
| Cyproterone acetate | (de Voogt, 1992; el Etreby et al., 1987; |
| | Varenhorst et al., 1982) |

### Nonsteroidal Antiandrogens

| Name | Reference |
|---|---|
| Bicalutamide | (Iversen et al., 2001; Iversen et al., 2000; |
| | See et al., 2002; Tyrrell et al., 1998) |
| Flutamide | (Brogden & Clissold, 1989; Sogani & Whitmore, 1988) |
| Nilutamide | (Davis et al., 2005) |

### Small-Molecule VEGF Antagonist and Inhibitors

| Name | Reference |
|---|---|
| CP-547,632 | (Beebe et al., 2003) |
| PTK787/ZK22584 | (Drevs et al., 2002) |
| SU5416 | (Shaheen et al., 1999) |
| SU6668 | (Fabbro & Manley, 2001) |
| SU11248 | (Sakamoto, 2004) |
| Thalidomide | (Baidas et al., 2000; Eisen et al., 2000; Eisen, 2000) |
| ZD6474 | (Ciardiello et al., 2004; Ciardiello et al., 2003) |

### PKA/CREB Inhibitors

| Target | Inhibitor | Reference |
|---|---|---|
| PKA | H-89 | (Graziani et al., 2002; Kaufmann et al., |
| | | 2002; Manna & Frazier, 2004) |
| | KT5720 | (Caraglia et al., 2002; Kim et al., 2002) |
| | PKA | (Cvijic et al., 2000; Graziani et al., |
| | inhibitor | 2002; Kaufmann et al., 2002; Manna & |
| | peptide | Frazier, 2004) |
| CREB | K-CREB | Lung cancer (Linnerth et al., 2005); |
| | (a dominant | melanoma (Aucoin et al., 2004; Jean et |
| | negative | al., 1998; Xie et al., 1997); gastric |
| | construct | cancer (Pradeep et al., 2004); |
| | to CREB | hepatocellular carcinoma (Abramovitch |
| | | et al., 2004); acute myeloid leukemia |
| | | (Kinjo et al., 2005; Shankar et al., 2005) |
| | single chain | Melanoma (Jean & Bar-Eli, 2001; Tellez |
| | Fv fragment | et al., 2004) |

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A method of treating a human patient having one or more tumor cells, comprising the step of implanting at or around the site of one or more tumor cells in the patient a cell population comprising one or more prostate cancer cells characterized in that the one or more prostate cancer cells have the propensity of metastasizing to skeleton and soft tissues which represent one or more lethal phenotypes of human prostate cancer.

2. The method of claim 1, which elicits an inflammatory response against the one or more tumor cells.

3. The method of claim 1, which elicits an immune response against the one or more tumor cells.

4. The method of claim 1, wherein the one or more tumor cells relate to a solid cancer.

5. The method of claim 4, wherein the solid cancer is selected from melanoma, pancreatic cancer, liver cancer, colon cancer, prostate cancer, and breast cancer.

6. The method of claim 1, wherein the step of implanting at or around the site of one or more tumor cells in the patient a cell population comprises the step of implanting at and around the site of one or more tumor cells substantially simultaneously in the patient a cell population comprising one or more prostate cancer cells characterized in that the one or more prostate cancer cells have the propensity of metastasizing to skeleton and soft tissues which represent one or more lethal phenotypes of human prostate cancer.

7. The method of claim 6, wherein cells around the site of one or more tumor cells comprise at least one of endothelial cells, inflammatory cells, bone stromal cells, prostate stromal cells, and cells in lymph node.

8. The method of claim 7, wherein the cells around the site of one or more tumor cells provide a host environment for the one or more tumor cells, wherein the one or more tumor cells interact with the host environment, and the interaction between the one or more tumor cells interact with the host environment controls tumor progression and metastasis.

9. A method of preventing a human patient from having one or more tumor cells, comprising the step of immunizing at or around a target site of one or more tumor cells likely to grow in the patient a cell population comprising one or more prostate cancer cells characterized in that the one or more prostate cancer cells have the propensity of metastasizing to skeleton and soft tissues which represent one or more lethal phenotypes of human prostate cancer.

10. The method of claim 9, wherein the one or more tumor cells relate to a solid cancer.

11. The method of claim 10, wherein the solid cancer is selected from melanoma, pancreatic cancer, liver cancer, colon cancer, prostate cancer, and breast cancer.

12. The method of claim 9, wherein the step of immunizing at or around a target site of one or more tumor cells likely to grow in the patient a cell population comprises the step of immunizing at or around a target site of one or more tumor cells likely to grow substantially simultaneously in the patient a cell population comprising one or more prostate cancer cells characterized in that the one or more prostate cancer cells have the propensity of metastasizing to skeleton and soft tissues which represent one or more lethal phenotypes of human prostate cancer.

13. The method of claim 12, wherein cells around the target site of one or more tumor cells likely to grow comprise at least one of endothelial cells, inflammatory cells, bone stromal cells, prostate stromal cells, and cells in lymph node.

14. A pharmaceutical preparation for inhibiting metastasis of cancer cells, comprising one or more prostate cancer cells characterized in that the one or more prostate cancer cells have the propensity of metastasizing to skeleton and soft tissues which represent one or more lethal phenotypes of human prostate cancer in a biologically compatible medium.


SEQUENCE LISTING (6 sequences)

SEQ ID NO: 1 - DNA, 946 bp, Homo sapiens
gagctcggatccactagtaacggccgccagtgtgctggaattccccttctgcagggtcaggaggagaatcgtggggccaggagggcagaggcacactccatcttcgtgctcctcacaggccctgctccctgcctgctaagacacagggaagggggcccccacctcagtgcctccctcccttccctgtgcctgtgtacctggcagtcacagccacctggcgtgtcccagaaaccaaccggctgacctcatctcctgcccggccccacctccattggctttggcttttggcgtttgtgctgcccgaccctttctcctgtccggatgcgcagggcagggctgagccgtcgagctgcacccacagcaggctgcctttggtgactcaccgggtgaacgggggcattgcgaggcatcccctccctgggtttggctcctgcccacggggctgacagtagaaatcacaggctgtgagacagctggagcccagctctgcttgaacctattttaggtctctgatccccgcttcctctttagactcccctagagctcagccagtgctcaacctgaggctgggggtctctgaggaagagtgagttggagctgaggggtctggggctgtcccctgagagaggggccagaggcagtgtcaagagccgggcagtctgattgtggctcaccctccatcactcccagggcccctggcccagcagccgcagctcccaaccacaatatcctttggggtttggcctacggagctggggcggatgaccccaaatagccctggcagattccccctagacccgcccgcaccatggtcaggcatgcccctcctcatcgctgggcacagcccagagggtataaacagtgctggaggctggcggggcaggccagctgagtcctgagcagcaaagggcgaattctgcagatatccatcacactggcggccgct

SEQ ID NO: 2 - DNA, 1468 bp, Homo sapiens
gtggcacatatacaccatggaatactatgcagccataaaaaatgatgagttcatgtcctttgtggggacatgtatgaaattggaaaccatcattctcagtaaactatcacaagaacaaaaaaccaaacaccacatattctcactcataggtgggaattgaacaatgagatcacatggacacaggaaggggaacatcacactctggggactgttgtggggtggggggagtaaggggagggatagcattgggagatatacctaatgctagatgatgagttagtgggtgcagcacaccagcatggcacatgtgtacgtatgtaactaacctgcacacaatgtgcacatgtaccctaaaacttaaagtataataataaaaaaaattaagagaaaaaaagaaaaaaaatgatattcattaatttttgatttctcaagcagacttcgcaactggaggaagaataaaatgactagactaggagaatatgcaaactattaagctagatttccctttataaattaaaaaattagtactttagtttatcaatccattctttgtggtgttggtttcatgaatcatttcaaaaacaatggatcactcctgctagctctagtcattttgttattctcataggaaaaaaattaaatatgaaaatgaatagaaaagatatatatagaagcccaagaaaaatcagctgacctcacatgcacgacaggaaggccacataaatggacaatatacagagatttaatttacaaaacaaaatataaaatctgcctctcagtggtatgattctcaaaagttctaacttttatactcagcatcatgttttagcaactatatgttacaaagtctgaccgacttaatcatatcaactttaatttatgagtcaatgaagtatatttcaggaggaaacatcaaatgatattaaaatattgatggttcatctgctggtttcccttattatttagtttttctttctttttttagctaaactaatgtaaaagttatatctaatgacagcaagctttcctttctttcgacatagtgaaaacttgtgtaattatgaaatttttaaaaggttaaagcctttgttatttattttaattcaaatccagtatattattatacatattcggagcccaaactattcatcttcatctaaaccttcaattaaattccacaatgcaaacctcttggctctagaatcacgtttcttgtttattcaactgagcctgtgtcttgaaaaagtgttgaagtttgggggttttctggtgagaatccacgttctgacatcaccttggtcgtgacagtgattggctgttggaaggcaaagaagagtttatagccagcaagagcaagtgaatgagtgagtgagagggcagaggaaatactcaatctgtgccactcactgccttgagcctgcttcctcactccaggactgccagagggtaagatttaatagaacaacgg

SEQ ID NO: 3 - DNA, 2200 bp, Homo sapiens
  modified_base (495): a, c, g, t, unknown or other
gggaaggagacaatagtgtcaacttgggattgcctaaggcaacaacagagcaaaacaagaacgctttggttctctgggtctctgtccctgattgcatagcgggtcattgttgggaaatatttcctcacctggcattccaagaaatggtgagctccacagctgtatatagtcctgtcattaaatacaggagtgttctatcccgctggaattaagaaaattggtagaaccagattgtggtctgaaatcttttttcagaaatgctgccatcgtgtggcactgcggagctatgaccagaagagtcctgtaaagggtcgtatggttcatctcaagatggctgggctccagcataatctattcctataattaattctagcttcatattgaatcattcccgtgggcacagagtaaactacagtaaatcctgtggaaattttgttgtttttagaattttcggacttccctccactaaattgacaacatgacacgcttatgcgngtatgtttaaaggaaaaaaatagtttttagaagcagaaaaaagaagtctattttgcaactttataatctgtgtgcttnctattttatagagatagtcgtcatcttacttattaaaatgggtgcttattacctacaaaccaatcatatcaattcatctggaatacatccaatttaagggagacatatttccccctaccaaatgttcatgaaacctatgaattagctatacactatcactgcaagacattatttaatctatatttatattaaaagtaatatttggcaaaaggaagctgacactttaggactaataaaaaccacaattacttttgcagcaacctaataataaataggaccatttatttttcatctcaattacacacaagtcttaacaataaaggtgtaaggtaaataaatagtgcaatctgcatttcacaactgagaagcaaatgaagataagtaatctcaaggcaatattaaatattttaaaaggacccagagctctgctatccctgaattctgctctaatattcggactttccctgtaattttctttcattcagacaccttttaaatacctagtaaagtgttttttaatacagaaatttttaaaaatgtttttctttttaagtggcctactttacataccttgggagaaaaactagaaaaaaagatgattccaaaatcgaatctgttcctttagaaatgtgcaaaatttccttattgatgcatacaatttaaagatcttacgtctactctcattttaataacctgttcttttaaaggacattacaattcgtgactgcctgcccctcttaaaaatttcataatagttaacacacatatagtccttaagatacgcagagcatttgcatctaatatgtgctaagcattgctagtttaacatactaattcatttaaacccctcaaaaaccccatgacctaggtaatagtattgcatttcatggatgagggaacaaggataggtaggctgggcgatttgcccaaggttgcacaggtcagcagtgacacagcggaattcagaaccacggtctggctcctgaagcagccctctcaagcagtcatccttctctcagtcagaaactgctttacttctgcaacatctagaataaattaccattcttctatttcatatagaattttatattttaatgtcactagtgccatttgtctaagtaacaagctactgcatactcgaaatcacaaagctaagcttgagtagtaaaggacagaggcaagttttctgaactccttgcaggcttgaacaatagccttctggctcttcaataagtacaatcatacaggcaagagtggttgcagatattacctttatgttacttaaaccgaaagaaacaaaaatccattgtatttaattttacattaatgtttttccctactttctccctttttcatgggatccctaagtgctcttcctggatgctgaatgcccatcccgtaaatgaaaaagctagttaatgatattgtacataagtaatgttttaactgtagattgtgtgtgtgcgtttttggtttttttttgttttaaccacaaaaccagagggggaagtgtgggagcaggtgggctgggcagtggcagaaaacctcatgacacaatctctccgcctccctgtgttggtggaggatgtctgcagcagcatttaaattctgggagggcttggttgtc

SEQ ID NO: 4 - DNA, 2313 bp, Homo sapiens
gaattccttgtactttttttcccttctcagttctgcacttaactcgtctaaaaaaattaaaaaagaatttaagaaaccacaaagctaagctgggtgcggtggctcacgcctgtaatcctagcactttgggaagccaaggcattcggattgcccaagctcaggagttcgagaccagcctgggcaacatgttgaaaccccatttctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccaacaaaactttttgaggaacaaagggaaccaggtattttattaattctcatacctccagagtgttaggcacaaaataaacattcaaccaagacctgttgcactgagcagttcatatataacaggagtgacccaagttgaaacgtagaatcagccctctcataccactttttgccaggtgatcataggcaagttacttagcatctatgtttccttattattaaaatggtcataattacaatgcctaagataagggggttgctgtgaagattattaaatcctcagtaaactttggctattgttactcctatgattatcatcaatatcatcaattaccttatctgttcaatactggtggcacaggtccaccagctagatgtctaatcccttatgtgtctattagtggtacaagtggagtttgagtgggatttttttttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctgcccctgggggccattgggtaattccttggagtctccaggcctcacttgccctctgaacaagaaagaggcctgttctggtcatccctccagcctgtccagccctggcactctgtgagtcggtttaggcagcagccccggaacagatgaggcaggcagggttgggacgtttggtcaggacagcccaccgcaaaaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgaggtatgtgtgacccccgcccagcctttcccttctatagttgcaccaaccccgacacccccgttcacgccgtcagctcgtgtgcaagggagggaagctctgctgaggatgcgcctctcctcccggctccatcacggctccccttaagagcatggccctcggtcctgtctgcctgttgcttttcagaaggtggactcactgtgtaactttgtcttcccttacaggtttacaggaaaataatctcactatgttcttcgggggagcattttctcactctctgtttttctctgtgtctgtctctggtttcagaggctgcctgcctgtcctctttgctccctttgcaaatgtggcagcctcctcctttcctgggaatctgatcccatcacagctgccacagggacctggccagcaaccggagtctgtcctccagatctcggtcaggggttctgttttccaaaaagggactttgcagaacaatcagttgatctctgaaagggaaagggggaggcttcaccattaatccacacctctgggaagcttctgttttcctctaattctcctcactcccaaacaccaccttccgtccccccaatacacaaatttcagcaccattctgcctgaaatggcaccatcacaacctcagtcttgggttaggtgttgttcctgtcctgagttccttgggatggtaaacacaggcagtagcccttagtttatctagatctgaaaacccagacatcagatatcgtcaaccaagacatgggtgtaatgggaggtggagtgtgctgggggagatattctcagaagggggaaagggggaagggaagagggagagaattc

SEQ ID NO: 5 - DNA, 4255 bp, Homo sapiens
acaccaaatatttataaatataactcacacaaataaaacctctttggtgttctcaaaattttgaagaatgtaaaaggtttgaaaattgctgatctagcaaatgactgaacatgaacagctatagtatttgtacctgcccagcagtgcagcaattccttatccttctcatatctgcactttaattttcctttgacaaatatctctccctcctctcagcccatgacatgaggttcacatggggttaacttaattccctggctcaaaggaaaggtattaaattcagacttgtatccaaccattcctgaagctagacttagccctatttttcaataacatgaaccaatcaattttcacatgagtccaaaataattctatgttaatacactaaggtactaggaaatatagtttgagaaatgttgatccaaacattgtgttatttacagtggagtattgacataaactttgaatcttcaaatatgttctggtgtcttggcatctcttaatacctattagcttacaaggctttcactcaactattttataattttgataatgacttaattgattagttgatatattgttaaaataaatatattaatgaatttatgataaataaggcagataaataagacatgcaattaggaagacatgttaaacaaattgttataataatacaatcactctcagcttaggatagctcctggccactttctctctgggtggtttttactctgggagtagtttaaatcattatctagtagtagtttaaagcattatctttgcctaagagctttcgctgactccccacatttgcattgtactaagagttttctctgactccccacataggtctagaccctagtattataagattctcattgtacttgcactttgccttcaaagtactaatcacggttttgttagtgatttgtgtgatgatttgttgaatctttttttttttcccactagggtgtaagccccatgttccatcttgatcaccatgtttctagcccagtgctggcatatagtgggttctcactaatatatctgtagagtaaatgaagaaatgcatgagtgacatgacaggagaatttaaggatgccatgggagcataaaacagagggagccacctgggtgaggagagctgagaaagacttctggagaggcgacatttgagctgagaaaggaaagacaagtgggagagtcctccaggtgtagaagttggagagatgagcgctccagttaggtagtatttgaagctgatgtagaaaaggagtcttgagccagcttgtgaaggactattggagagttttatttttatttttatcttttttttaatttttgagacagaatcttgctttgtctcccaggctggagtgcagtggcatgattgtagcttactgcagcttcgacctcctgggctcaaacaatccacctatctcagccttctgagtaactgggaccagagatgtgcaccaaaatgcctggctaatttgttcattttttgtaaagatagggtctccctatgttccccaggctattctccatctcctgggctccagtgatcctcacgcctcggccacccaaagtgctgggattatagaagtgaaccactgcgcctggcctattgaaggtttttaatcttcagagtttcgactttatcaacaacacttagaagccaccaaagaattgcaggtatggaaatgacatatacttttgcttttagaagaaaatcctgatcagtgtgcacagaattcttcagggggcaagtgtgattcattctgataagatatagcatggcttagactgggagactggcagaggctttgaagatttctttgctcaaattttattcagcaagtatttaccatgcacctactatagcaggcaacatttttaggaaatggtgaatgttacagaggtgaataatacagcaagagtcgttgaacatatggagtttatctattagttggggagtgaatgttgacaaaggaataagtaaatacataggcaagaaagatacattacctgtgaaacagcagcaggtagactgacagtggagtatctaatacagcctatggaagccagaagatagtgggatgacatttttggagtactagtagaaatgtcatatgaagaactctgtaggaatgtaacatacggtcccatatatgaagctcctgggtcaagtatacctgaacataattcagggatttgagggactttcttgtaacctgaggatcaagatgtcaaggaattaaaaacatgtataaaacattgttgtataaaaacccattaaaaagaatggaagacactatagtaaaatcattgtgggtttagttgttataacacattttaaaaatctttgatcccaatcaatatttataagaaagaagaaatatggaattatttcctgagtcaaggagcagggagagaatgaggaagaagaggaggaggaggagggggaggaggagacaataaacctacttcccaaagttaacaaacaaaaagtgggaagaggtcaaagactacaaggagtagaattaacgtcaattgtttctatgtttgagtctgaaaattttttgtcccttctccaccaacctatatattgatacacatataaatgctaaaggcatttttgaatttgaacagatcattttctttgtatggctgcctttaaaaaaaattcaacctggtcactcttcctcaacatttactgaggtctaagtgttcaatttagaacacatgctttaataactcagagacctgtcatttgtcacaaatcttgcctagagaaatactcattagcgaattaggcagaaagaggatgcaaaataaaaaggcacagtagtcccctgatatccatggaagactggttccaggacaccaccaaacccctccccgcaaataccaaaatccatggatgttcaagtttcttaacatatcatggcatagtatttgcatttaacctacacacatcctcttgtacacttgaaattatctttagattatttataatacttaatagaatgtaaatgctatgtaactagttgtgtatcatttaggaaatgatcacaagaaaaaaagtctacagatgttagtccagacacagccatcctttttttttttttcaaatatttttgatctgtggttcattgcatccacagatgtggaacccatggatactgtgggctaactgtattaataaaaaagtggaaacatcctaagtttcatgggtgtttaaattggtcagcaacttccttctgaagaagtatcagaatttgtgagcaatgttaatatttttgttttctcactaagagccacagttctgaatagaggtttttaaaaagccctagcaaggtttctttagcaatgaaactaacatttaactgtatcatcagcttcgtgttacatctctttcctgactgttgggtgagccctcctcggatgcttgcttctggctacacgcccctttacccttttctctgcactgttttcatctttataaagtcagagttggtgtctataggctctctactgccacattcaagacctgcctcgctcaatgtcaccttcaagatgcagaaatagggatttgggaaggggattgtgaaattttcgaagtcttccaaaatactttgagaaactatatttggaagcactttggggggagaggttggacaggaagggtcttcagagatcatcaaatttaactttctaaatcctaaggaggaaaccgagactccaggatgtgaagtcccttctctaccaaactagaatggatgcaggaggaatgtctgaggtgcaatccttatcctttagcaaaggtgtcctctgcgtcttctttaacccatctcttggacctccagaaagacagctgaggatggcaaggggagtctggaaccactggagtagcccccagcctcctccttggagggcccccatgaaggaggcccttcagtgacagagattgagagagagggagggcgaaaggaaggaaggggagccagaggtgggagtggaagaggcagcctcgcctggggctgattggctcccgaggccagggctctccaagcggtttataagagttggggctgccgggcgccctgcccgctcgcccgcgcgccccaggacccaaagccgggctccaagtcggcgccccacgtcgaggctccgccgcagcctccggagttggccgcagacaagaaggggagggagcgggagagggaggagagctccgaagcgagagggccga

SEQ ID NO: 6 - DNA, 6905 bp, Homo sapiens
tctagaaaataattcccaatattgaatcccaaagaattcaacatttgggctgtcgtttgaaagataagttgaatttggtcatgaaggaagagaggggggatacaatttcagtaaaaggtaacagcaaggtccaaagacagtcaggtcttcagtagtatggagtatattcagagggagccaagatgtctgatgtgaactaaaaagattggtggttggtaggaggaagaggtgtgagaagaggctgtaaagaaaaattgaaacttgattgtgatggactttaaaggctaggctatgggacttggacatgaatctgcaggccagtgtttgcagactggcgcccataactgtctatcacagcaacacagacatgtgttgtttggcctgcagaggtttggcctgcatgatgattttaaaccatctgaattagtagccatcattttcaaaaatcaagagatgccacattaaaatatggaatgctgctgttcttgaaaataatgaaacatctggaacattgaggccacattcctgactgacagcaatcagttggagctgcgtagtgactgcccactttacatggggcatctgatccctagtcgattacagctgccaccacttccctttatctctctaataccaagctcttttcactcatttttgttacttaagagatatttgggtttgaaacctctgatgcaggtaattgagggttatagagcagaggacagatgctatcagagttgtcttttaagaaagaaccctctgttcttcattttgttgaagatagcctggaagagggcagccaggggagaagttagggctggagctatgagaaagcataagatgagatgatggcttcaacattgaggacagaaagaatattgagatgagaaagtagtccatataagcatctatgcaaaggaaatagcagatgtcctcaaatcagcagaggcaacaactctgaaagtttattcataagcccctcttttcatctccaatccagttcaaatgtaattatttaaattgttcttcactctccttcctggatcatgaatgagctccttaaatgcagggtccacagtgtcctattcatcagtgaattccaagtgcctagcacagagcctggcaaatagtaaatgcttaacaaatattcgttcagtgcatgaattggagtgattctctactttgcctcataagttgaaaaaaggtttattacatacctaaatatgctgaaatcacagggcatttggcaaccccccaaaaccaaaactcccagtttggaaacagaattttaattctgtgaaaataaaatccattcatttattcaaaaaatatttattaaacaatgaccatgtccacaccaggctgagtcctaaggattcaatgatgaacaaaaaccaacatgattcctgctcttaggaaacatacagttcagtgaggaaaacagattgtgagaagtcctccaacaaatactgggtgctattaaaatatattaaaaggtgagtgggtgagggacttgagctagcctaggtggttcaggaagtcttcctggatgtgctgatatgcataggcattaactagataaatagagagaaggatgaaccaacattgcaggtagagggaacagaatatgcaaaggcaggaaggattatggagtcgttggaggacctgaataaaggcccagtgtaagtggatctcagaaaacaggaggaaaggtgtatgagatgagatcagagaggcagatcatgtggggtatggttaatgttttggacttttctattaagagcaatggggagacagtgacaggacttaaacggggaaataatatgaccagattaaactttctaaaaaaccctctatgcaaatatatattgagagttaattattgacaaagattcaaaggcaacaaagtggagagagaatagtattttcaaaaaatggtgccaaaacaataggacatctatattaaaagttgggtatctgtctacaaaacttaattcaaaatggatcacagacctaaatgtaaaactgaaagctatacaacttctggaaggaaaacacagatgggaatctgtgtgatcttgagtttgaaaatgatttattatatctgacaccataatccgtaagttaacataattcataagtgaacaaagtgatgaactggacttcatcagaatttaaaatgtttgtgcttcaaaagacactggtatgataatgaagacaaactacagataagatattgttgaatcatatttctgataaaggaattgtggctcagaatacataactctaaacccccataataaattacaagtagcccaattaaaaaaaaaaaaagagaaaaaatttacagtcttcatcaaagaaagtatcaattgtaaaataagcacatgaaaaatgctctgcatctttattcatggggggatgaaataaaaattaaatgggaaagacacctctaattagaatactaaaattaaaaagactgaccataccaagtattggtgaagtggaaatgtaaaatgatacaatcaacttaggtagatgatttggaagtttcttacaaaagtaggtgtatacctaccctgtgactcacccattccatggctaagtatttacctgagagaaatgaaagaatacatccatacaaagatgtttatacaaatatttatagcagttttatttgtagtagccccaaactgaaaagaacccaaatgtccatcaaaagtgaatggataaacaaagcgtggtacagcaatgcaatagaatactacttagcaataaagaagaatgagctagtgatatacataacagcttaaatgtacatcaaaggcattgtgctcagtgaaagatgcaagtaaaaaaaaaaaagagtacatgctgtatagttccattgacataaaactctggaaagtgaaaaacagtctatactgacagaaagcagatcattggttgcctgaggaggaggagtataggagaggtggagggaaaatgtacaaagtggcacaataaaaacttttggaatcatagatatattcactatcttgattgagtgatgatttcatgagtgcacgtgcgtgtgtcaaaaatgatcaatttatgcaactttaaatatgtgcagtttattgtatatatcaattatacctcagtacggctattaaaaagaaaccctctggctgcacaatgcagaactgattctaggaaagagtggagggaggatgaccatttacagtgctccaggtggaagagaacggtgccttctggaagtgaactaggttggcaacaacagagatgaaataaatgggcagatgtgtgagatacttaggaaataaaacccgatggtcaccattttccaaaggtcagctcatcctggctttccagagcaaagagctagggaagactttattaataaatccctcttgaagttgcagaggaagcttatagcagaaacttactctcaacctgactaatctgagagaacacctctggttccatttgattactaaaaaactgcaaagaacaggaggagaaagaagaagaaagctggtacaaacagtgaacttatataatattaatcaataattgtctcttgttcttaaaagcaatgggaagaaaatgagatttgagctggaagatcagagttcaaaatccaaataaagtatatggccctaatatgcttatagtagttaacctttcctgataatgatataattgttgacagcaccatctttaaaataaaataacatagtaatccttcagatttgtagaagatctttcctgtttacaagtttgttctatacacattatgtcttttaaatgacacactagccttctgagggtaacttatattggcaacagttttcagatgtggaaactgtgaagacaatgttggtgatgtggaagcaacataaactttggagtctttcagacccaggtttgaatgtcagactgctttttattcagagtaacttcagagcattatttctcaccttaattttttttcaggcctctttgtgtctatgtgtcctcttcactcctgtccattgtttcttcagtgatttttgccaccttccttcactgttagtgtgtagacacatagttctcctggctctgagagcctatgttaattccattctaccatcctgccacggcccactcaattcctattgagcaatgctagttgaaagttgtggtgggattaaatgttgcaatgagtattcaaatgaggttgaagtatctacgcattctacttacatatggtgaggtatattcaaggaagctgtagccattaaaatctcaggaaataatttttcacctcctcaggtgaaagggtcttcaggcctttgtgttctggaaggttcatttatagccatttcccaaatgacaatgcgattgatgagtctagagtctagctcaaatagcaatggactggaagactagtttaggttttactaatgtggaacatagaacaaattatgtccttgtttcagcctgttcatctgtgaaatagagcctatcatatccagtcttccttgcctttaggtttgagttaccttctttggtcaaggtaagtaaatgcctatgatgtttggctgtgcacaagataaagctacaacaaagctacaacccatcttttctctgtagaagactcaaaaagcaaaagagacccaggaaaatctcggaatgacttttggaacagagagcctccccagaatcagaagtcaaggaatttaaacatagggaaggcccaggtctctactgacataaaggaaagatgttttcttataggtttcacgtttacattttctctctcttgatcccattcccacttgcatctgccacctttacacagggcttatgggacctcctccacaaaagagcagttgcagtaacccacatcatcctctacgccctggctgtccatcaagaggcgaaaagcagccctatataggttctatccttggatagttccagttgtaaagtttaaaatatgcgaaggcaacttggaaaagcaagcggctgcatacaaagcaaacgtttacagagctctggacaaaattgagcgcctatgtgtacatggcaagtgtttttagtgtttgtgtgtttacctgcttgtctgggtgattttgcctttgagagtctggagagtagaagtactggttaaaggaacttccagacaggaagaaggcagagaagagggtagaaatgactctgattcttggggctgagggttcctagagcaaatggcacaatgccacgaggcccgatctatccctatgacggaatctaaggtttcagcaagtatctgctggcttggtcatggcttgctcctcagtttgtaggagactctcccactctcccatctgcgcgctcttatcagtcctgaaaagaacccctggcagccaggagcaggtattcctatcgtccttttcctccctccctcgccccaccctgttggttttttagattgggctttggaaccaaatttcctgagtgctggcctccaggaaatctggagccctggcgcctaaaccttggtttaggaaaccaggagctattcaggaagcaggggtcctccagggctagagctagcctctcctgccctcgcccacgctgcgccagcacttgtttctccaaagccactaggcaggcgttagcgcgcggtgaggggaggggagaaaaggaaaggggaggggagggaaaaggaggtgggaaggcaaggaggccggcccggtgggggcgggacccgactcgcaaactgttgcatttgctctccacctcccagcgccccctccgagatcccggggagccagcttgctgggagagcgggacggtccggagcaagcccacaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcgcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaacaagaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatg